(19) United States
(12) Patent Application Publication
(10) Pub. No.: US 2026/0007122 A1
(43) Pub. Date: Jan. 8, 2026

(54) AAVR KNOCKOUT MOUSE IN COMBINATION WITH HUMAN LIVER CHIMERISM AND METHODS OF USE AND PRODUCTION OF THE SAME

(71) Applicant: Avachrome, Inc., Durham, NC (US)

(72) Inventors: Maria DE LAS MERCEDES BARZI DIEGUEZ, Houston, TX (US); Francis Peter PANKOWICZ, Houston, TX (US); Karl-Dimiter BISSIG, Houston, TX (US)

(21) Appl. No.: 18/685,160

(22) PCT Filed: Aug. 30, 2022
(86) PCT No.: PCT/US2022/075633

§ 371 Date: Apr. 21, 2025

Related U.S. Application Data
(60) Provisional application No. 63/238,386, filed on Aug. 30, 2021.

(51) Int. Cl. A01K 67/0271; A01K 67/0276; A61K 49/00; C07K 14/705

(52) U.S. Cl. CPC A01K 67/0271; A01K 67/0276; A61K 49/0008; C07K 14/705; A01K 2207/12; A01K 2207/15; A01K 2217/075; A01K 2227/105; A01K 2267/03

(57) ABSTRACT

The present disclosure provides an immunodeficient or immune-impaired chimeric non-human animal with a deletion or impairment of adeno-associated virus receptor (AAVR), comprising human hepatocytes, methods for preparing the chimeric non-human animal comprising human hepatocytes and methods of utilizing the chimeric non-human animal comprising human hepatocytes to evaluate transduction efficiency of adeno-associated viruses (AAV), and determine mechanism of inhibition/modification of AAV transduction in human hepatocytes.

### CROSS REFERENCE TO RELATED APPLICATION

This application claims priority to and the benefit of U.S. Provisional Application No. 63/238,386, filed Aug. 30, 2021, the content of which is incorporated herein by reference in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is AVCR-003_001WO_ST26”. The XML file is 35,504 bytes in size, created on Aug. 24, 2022, and is being submitted electronically via USPTO Patent Center.

### BACKGROUND OF THE INVENTION

Adeno-associated virus (AAV) vectors are used as viral delivery agents for gene therapy and for generating human disease models. Chimeric humanized mouse models have been used by researchers for determining the transduction efficiency of AAV vector serotypes and their variants, but are limiting in value because most AAV serotypes preferably transduce mouse hepatocytes over human hepatocytes. The present disclosure solves these needs in the art by providing a Adeno-associated virus receptor (AAVR) knock out human liver chimeric non-human animal model and methods of using the same to evaluate AAV transduction efficiency.

### SUMMARY OF THE INVENTION

The present disclosure provides a chimeric non-human animal comprising human hepatocytes, wherein the chimeric non-human animal comprises: a) a T-, B- and/or NK cell deficiency or functional impairment that allows re-populating with human hepatocytes to establish human chimerism in the liver of the non-human animal; and b) a deletion or mutation of Adeno-associated virus receptor (AAVR) resulting in deficiency or functional impairment of the non-human animal AAVR.

The present disclosure provides a chimeric non-human animal comprising human hepatocytes, wherein the chimeric non-human animal is a IL-2Rg−/−/Rag 2−/− chimeric non-human animal, and wherein the chimeric non-human animal comprises a deletion or mutation of AAVR resulting in deficiency or functional impairment of the non-human animal AAVR. In some embodiments, the chimeric non-human animal further comprises Fah−/−. In some embodiments, the chimeric non-human animal does not comprise a transgene. In some embodiments, the transgene is an antibiotic resistance cassette.

The present disclosure also provides a method for preparing a chimeric non-human animal comprising human hepatocytes, the method comprising: (a) a T-, B- and/or NK cell deficiency or functional impairment that allows re-populating with human hepatocytes to establish human chimerism in the liver of the non-human animal, wherein the non-human animal comprises a deletion or mutation of AAVR resulting in a non-functional non-human animal AAVR; and (b) transplanting human hepatocytes into the non-human animal. In some embodiments of the method for preparing a chimeric non-human animal comprising human hepatocytes of the present disclosure, step (b) further comprises applying a selection pressure.

The present disclosure also provides a method for preparing a chimeric non-human animal comprising human hepatocytes, the method comprising: (a) providing a IL-2Rg−/−/Rag 2−/− non-human animal, wherein the non-human animal comprises a deletion or mutation of Adeno-associated virus receptor (AAVR) resulting in a non-functional non-human animal AAVR; and (b) transplanting human hepatocytes into the non-human animal. In some embodiments of the methods of this disclosure, the non-human animal further comprises a Fah−/−. In some embodiments of the method for preparing a chimeric non-human animal comprising human hepatocytes of the present disclosure, step (b) further comprises applying a selection pressure.

In some embodiments of the methods of this disclosure, the chimeric non-human animal does not comprise a transgene. In some embodiments of the methods of this disclosure, the transgene is an antibiotic resistance cassette. The present disclosure also provides a chimeric non-human animal produced by the methods disclosed herein.

The applying of a selection pressure can comprise not providing nitisinone (NTBC) to the non-human animal of step (b) of the methods for preparing a chimeric non-human animal comprising human hepatocytes of the present disclosure. In an aspect, the method of preparing can further comprise removal of the selection pressure following step (b) of the methods disclosed herein. The removal of the selection pressure can comprise providing nitisinone (NTBC) to the chimeric non-human animal following step (c) of the methods disclosed herein.

The present disclosure also provides a method of determining transduction efficiency of an AAV vector in human hepatocytes, wherein the method comprises: (a) providing a chimeric non-human animal prepared by any one of the methods of the disclosure; (b) infecting the non-human animal of (a) with an amount of the AAV vector; and (c) determining the level of transduction of the AAV vector into the human hepatocytes and the hepatocytes of the AAVR KO non-human animal (non-human animal hepatocytes). In some embodiments of the methods of the disclosure, in step (c) the level of transduction of the AAV vector into the human hepatocytes or the non-human animal hepatocytes, is measured as: (i) percentage of the AAV vector-transduced human hepatocytes or percentage of AAV vector-transduced non-human animal hepatocytes, respectively, in the non-human animal; or (ii) percentage of the total amount of the AAV vector-transduced into the human hepatocytes or the non-human animal hepatocytes, respectively, in the non-human animal.

In some embodiments of the methods of the disclosure, the method further comprises: (d) selecting the AAV vector as efficient in transducing human hepatocytes if: (i) less than a pre-determined percentage of the total amount of the AAV vector is transduced into the non-human animal hepatocytes; (ii) at least a pre-determined percentage of the total amount of AAV vectors is transduced into the human hepatocytes; (iii) percentage of AAV vector-transduced non-human animal hepatocytes is less than a pre-determined value; and/or (iv) percentage of AAV vector-transduced human hepatocytes is more than a pre-determined value.

The present disclosure also provides a method of determining transduction efficiency of two or more non-identical AAV vector(s) in human hepatocytes, wherein the method comprises: (a) providing two or more chimeric AAVR KO non-human animal generated by any one of the methods of the disclosure; (b) infecting each of the non-human animals of (b) with an amount of the two or more non-identical AAV vectors, wherein one non-human animal is infected with one AAV vector; and (c) determining the level of transduction of the AAV vector into the human hepatocytes and the hepatocytes of the AAVR KO non-human animal (non-human animal hepatocytes) in each of the two or more non-human animals of (a). In some embodiments of the methods of this disclosure, the method further comprises: (d) comparing the level of transduction of each of the two or more AAV vectors into the non-human hepatocytes determined in (c); and/or (e) comparing the level of transduction of each of the two or more AAV vectors into the human hepatocytes determined in (c). In some embodiments of the methods of this disclosure, the method further comprises: (f) selecting one or more AAV vector(s) as efficient in transducing human hepatocytes if: (i) less than a pre-determined percentage of the total amount of the AAV vector is transduced into the non-human animal hepatocytes; (ii) at least a pre-determined percentage of the total amount of AAV vectors is transduced into the human hepatocytes; (iii) percentage of AAV vector-transduced non-human animal hepatocytes is less than a pre-determined value; and/or (iv) percentage of AAV vector-transduced human hepatocytes is more than a pre-determined value.

The present disclosure also provides a method of determining the efficiency of a systemic AAV vector-mediated gene therapy, wherein the method comprises: (a) providing a chimeric non-human animal prepared by any of the methods of the disclosure; (b) infecting the non-human animal of (a) with an amount of an AAV vector, at least a first time; and (c) determining the level of transduction of the AAV vector into the human hepatocytes of the AAVR KO non-human animal. In some embodiment of the methods of the disclosure, the method comprises: (d) infecting the non-human animal with an amount of an AAV vector, a second time; (e) maintaining the infected non-human animal of (d) for an amount of time; and (g) determining the level of transduction of the AAV vector into the human hepatocytes of the AAVR KO non-human animal. In some embodiments of the methods of the disclosure, the method further comprises: (i) comparing the level of transduction of the AAV vector into the human hepatocytes between the first and the second infection.

The present disclosure also provides a method of determining Adeno-associated virus receptor (AAVR)-dependent or AAVR-independent transduction efficiency of an AAV vector in human hepatocytes, wherein the method comprises: (a) providing two or more chimeric non-human animals prepared by any one of the methods of the disclosure, divided into two groups, Group A and Group B, each group comprising one or more non-human animals, wherein the Group A receives wild type human hepatocytes and Group B receives human hepatocytes comprising a deletion or mutation of AAVR resulting in a non-functional human AAVR (Hu AAVR KO human hepatocytes); (b) infecting the one or more infected non-human animal of each group of (a) with an AAV vector; and (c) determining the level of transduction of the AAV vector into the human hepatocytes of non-human animal of Group A and Group B, wherein the level of transduction in Group A is indicative of AAVR-dependent transduction efficiency and the level of transduction in Group B is indicative of AAVR-independent transduction efficiency, of the AAV vector.

The present disclosure also provides a method of determining Adeno-associated virus receptor (AAVR) dependent or AAVR-independent modification and/or inhibition of an AAV vector transduction into human hepatocytes, wherein the method comprises: (a) providing two groups of non-human animals, Group A and Group B, wherein Group A comprises one or more a T-, B- and NK cell deficient or impaired in function non-human animals, and Group B comprises one or a T, B- and NK cell deficient or impaired in function non-human animals, further comprising a deletion or mutation of AAVR resulting in a non-functional non-human animal AAVR, wherein the one or more a T-, B- and NK cell deficient or impaired in function allow re-populating with human hepatocytes to establish human chimerism in the liver of the non-human animal; (b) transplanting human hepatocytes into the one or more non-human animals of both groups of (a); (c) infecting the non-human animals of both groups of (b) with an AAV vector; and (d) determining the level of transduction of the AAV vector into the human hepatocytes and non-human hepatocytes of the non-human animals of Group A and Group B. In some embodiments of the method determining AAVR-dependent or AAVR-independent modification and/or inhibition of an AAV vector transduction into human hepatocytes of the present disclosure, step (b) further comprises applying a selection pressure.

The present disclosure also provides a method of determining AAVR-dependent or AAVR-independent modification and/or inhibition of an AAV vector transduction into human hepatocytes, wherein the method comprises: (a) providing two groups of non-human animals, Group A and Group B, wherein Group A comprises one or more IL-2Rg−/−/Rag 2−/− non-human animals, and Group B comprises one or more IL-2Rg−/−/Rag 2−/− non-human animals, further comprising a deletion or mutation of Adeno-associated virus receptor (AAVR) resulting in a non-functional non-human animal AAVR; (b) transplanting human hepatocytes into the one or more non-human animals of both groups of (a); (c) infecting the non-human animals of both groups of (b) with an AAV vector; and (d) determining the level of transduction of the AAV vector into the human hepatocytes and non-human hepatocytes of the non-human animals of Group A and Group B. In some embodiments of the methods of this disclosure, the non-human animal further comprises a Fah^{-/-}. In some embodiments of the method of determining AAVR-dependent or AAVR-independent modification and/or inhibition of an AAV vector transduction into human hepatocytes of the present disclosure, step (b) further comprises applying selection pressure. In some embodiments of the methods of the disclosure, applying a selection pressure comprises not providing nitisinone (NTBC) to the non-human animal of step (b). In some embodiments of the methods of the disclosure, the method further comprises removing the selection pressure following step (b).

In some embodiments of the methods of the disclosure, the method further comprises: (e) comparing the level of transduction of the AAV vector into the human hepatocytes and non-human hepatocytes of the non-human animals between Group A and Group B, (i) wherein the difference between the levels of transduction of AAV into the non-human hepatocytes and the human hepatocytes of Group A is indicative of both AAVR-dependent and -independent modification and/or inhibition of uptake of the AAV vector into human hepatocytes; (ii) wherein the difference between the levels of transduction of AAV into the non-human hepatocytes and the human hepatocytes of Group B is indicative of both AAVR-independent modification and/or inhibition of uptake of the AAV vector into human hepatocytes; and (iii) wherein the difference between the levels of transduction of AAV into human hepatocytes in Group B and Group A is indicative of AAVR-dependent modification and/or inhibition of uptake of the AAV vector into human hepatocytes.

In another aspect, provided herein is a chimeric non-human animal comprising at least two human tissues, wherein the chimeric non-human animal further comprises: (a) a T-, B- and/or NK cell deficiency or functional impairment that allows re-populating with one or more human tissues to establish human chimerism in the non-human animal; and (b) a deletion or mutation of Adeno-associated virus receptor (AAVR) resulting in deficiency or functional impairment of the non-human animal AAVR. In another aspect, provided herein is a chimeric non-human animal comprising of one or more human tissues, wherein the chimeric non-human animal is a IL-2Rg−/−/Rag 2−/− chimeric non-human animal, and wherein the chimeric non-human animal further comprises a deletion or mutation of AAVR resulting in deficiency or functional impairment of the non-human animal AAVR. In some embodiments, the chimeric non-human animal is a IL-2Rg−/−/Rag 2−/−/Fah−/− non-human animal.

In another aspect, provided herein is a method for preparing a chimeric non-human animal comprising one or more human tissues, the method comprising: (a) providing a T-, B- and/or NK cell deficient or impaired in function non-human animal, that allows re-populating with human tissue to establish human chimerism in the non-human animal, wherein the non-human animal comprises a deletion or mutation of AAVR resulting in a non-functional non-human animal AAVR; and (b) transplanting one or more human tissues into the non-human animal. In another aspect, provided herein is a method for preparing a chimeric non-human animal comprising one or more human tissues, the method comprising: (a) providing a IL-2Rg−/−/Rag 2−/− non-human animal, wherein the non-human animal comprises a deletion or mutation of Adeno-associated virus receptor (AAVR) resulting in a non-functional non-human animal AAVR; and (b) transplanting human tissue into the non-human animal. In some embodiments, the non-human animal further comprises a Fah−/−.

In another aspect, provided herein is a method of determining transduction efficiency of an AAV vector in one or more human tissues, wherein the method comprises: (a) providing a non-human animal described herein (b) infecting the non-human animal of (a) with an amount of the AAV vector; and (c) determining the level of transduction of the AAV vector into the human tissue of the AAVR KO non-human animal.

In another aspect, provided herein is a method of determining transduction efficiency of two or more non-identical AAV vector(s) in at least two one or more human tissues, wherein the method comprises: (a) providing two or more AAVR KO non-human animal described herein, (b) infecting each of the non-human animals of (a) with an amount of the two or more non-identical AAV vectors, wherein each non-human animal is infected with one AAV vector; (c) determining the level of transduction of the AAV vector into at least one human tissue in each of the two or more non-human animals of (b).

In another aspect, provided herein is a method of determining the efficiency of a systemic AAV vector-mediated gene therapy, wherein the method comprises: (a) providing a non-human animal described herein; (b) infecting the non-human animal of (a) with an amount of an AAV vector, at least a first time; (c) determining the level of transduction of the AAV vector into the human tissues of the AAVR KO non-human animal.

In another aspect, provided herein is a method of determining Adeno-associated virus receptor (AAVR) dependent or AAVR-independent transduction efficiency of an AAV vector in one or more human tissues, wherein the method comprises: (a) providing non-human animals divided into two groups, Group A and Group B, each group comprising one or more non-human animals, wherein the Group A is transplanted with wild type human tissue and Group B is transplanted with human tissue comprising a deletion or mutation of Adeno-associated virus receptor (AAVR) resulting in a non-functional human AAVR, wherein the two or more non-human animals further comprise a T-, B- and/or NK cell deficiency or impairment in function, IL-2Rg−/−/Rag 2−/−/, and/or Fah−/−: (b) infecting the one or more non-human animal of each group of (a) with an AAV vector; and (c) determining the level of transduction of the AAV vector into one or more human tissue of non-human animal of Group A and Group B, wherein the level of transduction in Group A is indicative of AAVR-dependent transduction efficiency and the level of transduction in Group B is indicative of AAVR-independent transduction efficiency, of the AAV vector.

In another aspect, provided herein is a method of determining Adeno-associated virus receptor (AAVR) dependent or AAVR-independent modification and/or inhibition of an AAV vector transduction into human tissues, wherein the method comprises: (a) providing groups of non-human animals, Group A and Group B, wherein Group A comprises one or more a T-, B- and NK cell deficient or impaired in function non-human animals, and Group B comprises one or a T-, B- and NK cell deficient or impaired in function non-human animals, further comprising a deletion or mutation of Adeno-associated virus receptor (AAVR) resulting in a non-functional non-human animal AAVR, wherein the one or more a T-, B- and NK cell deficient or impaired in function allow re-populating with human tissue to establish human chimerism in the non-human animal; (b) transplanting one or more human tissues into the one or more non-human animals of both groups of (a); (c) infecting the non-human animals of both groups of (b) with an AAV vector; and (d) determining the level of transduction of the AAV vector into human tissue of the non-human animals of Group A and Group B.

In another aspect, provided herein is a method of determining Adeno-associated virus receptor (AAVR) dependent or AAVR-independent modification and/or inhibition of an AAV vector transduction into one or more human tissues, wherein the method comprises: (a) providing two groups of non-human animals, Group A and Group B, wherein Group A comprises one or more IL-2Rg−/−/Rag 2−/− non-human animals, and Group B comprises one or more IL-2Rg−/−/Rag 2−/− non-human animals, further comprising a deletion or mutation of Adeno-associated virus receptor (AAVR) resulting in a non-functional non-human animal AAVR; (b) transplanting one or more human tissues into the one or more non-human animals of both groups of (a); (c) infecting the non-human animals of both groups of (b) with an AAV vector; and (d) determining the level of transduction of the AAV vector into human tissue of the non-human animals of Group A and Group B. In some embodiments, the non-human animal further comprises a Fah−/−.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

### BRIEF DESCRIPTION OF THE DRAWINGS

The patent or application file contains at least one drawing executed in color. Copies of this patent or patent application publication with color drawings will be provided by the Office upon request and payment of the necessary fee.

The above and further features will be more clearly appreciated from the following detailed description when taken in conjunction with the accompanying drawings.

FIGS. 1A-1B depict generation of CRISPR-KO strain of AAVR. FIG. 1A is a schematic drawing of AAVR gene with boxes depicting exons (exon 1 (E1) to exon 6 (E6)).

FIG. 1B depicts a gel electrophoresis picture of PCR amplification showing generation of homozygous and heterozygous AAVR KO mice using intronic primers. Wild type band (2.3 kbp) and knock out band (0.2 kbp) from homozygous and heterozygous animals generated, in the gel electrophoresis gel are as indicated.

FIG. 2A-2B depicts genotyping scheme and probes/primers for TIRFA (transgene free Il2rg−/−/Rag2−/−/Fah−/−/AAVR−/−) KO mice. FIG. 2A is a schematic drawing of the semi-quantitative PCR using Taqman probes. FIG. 2B depicts the primer sequences and reporters used in the semi-quantitative PCR.

FIG. 3 depicts transduction of human liver chimeric mice by different AAV serotypes. The x-axis depicts the identity of the AAV vector in terms of capsid (serotype/variant), promoter type and detectable/reporter gene, used to transduce FRG (Fah−/−/Rag2−/−/Il2rg−/−) human liver chimeric mice, as indicated. The y-axis depicts the percentage transduction of human hepatocytes (white bars) and mouse hepatocytes (black bars) in the human chimeric livers of FRG human liver chimeric mice. Data shown in each bar is mean±SEM of 5-11 sets of images for each chimeric mouse liver. FIG. 3 depicts data that is partially adapted from Bissig-Choisat B. et al., Nature communications, 2015.

FIG. 4A-4F depict transduction of human liver chimeric FRG mice by AAV8 and AAV9, adapted from Bissig-Choisat B Nature communications 2015. FIGS. 4A-4C show transduction of hepatocytes of FRG mice transduced with AAV8. FIGS. 4D-4F show transduction of hepatocytes of FRG mice transduced with AAV9. Transduced mouse hepatocytes are indicated by arrows and transduced human hepatocytes are indicated by arrowheads. FIGS. 4B and 4F are magnified representatives of the are marked by white square in FIGS. 4A and 4D, respectively (scale bar is 50 ∞m).

FIG. 5A-5L depicts transduction of AAVR knock out human liver chimeric TIRFA (transgene free Il2rg−/−/Rag2−/−/Fah−/−/AAVR−/−) mice by AAV9. FIGS. 5A-5F depict representative immunofluorescent staining panels from duplicate experiments showing transduction of hepatocytes by AAV9 in human liver chimeric mice with homozygous deletion of AAVR (TIRFA/AAVR−/−). FIGS. 5G-5L depict duplicate experiments showing transduction of hepatocytes by AAV9 in human liver chimeric TIRFA mice with heterozygous deletion of AAVR (TIRFA/AAVR+/−). FIGS. 5A, 5D, 5G and 5J depict panels showing staining for AAV vector transduction (dTomato). FIGS. 5B, 5E, 5H and 5K depict panels showing staining for human hepatocyte FAH (human LDHA). FIGS. 5C, 5F, 5I and 5L depict overlay of staining for AAV vector transduction and human hepatocytes.

FIGS. 6A and 6B depict transduction of AAV8 in humanized TIRF and TIRFA mice. Humanized TIRF (transgene free Il2rg^{-/-}/Rag2^{-/-}/Fah^{-/-}/Aavr^{+/+}) (FIG. 6A) and humanized TIRFA (transgene free Il2rg^{-/-}/Rag2^{-/-}/Fah^{-/-}/Aavr^{-/-}) (FIG. 6B) mice were injected with 1×10¹² GC/mouse of AAV8 carrying a td-Tomato expression cassette. Mice were euthanized after 72 hours, and livers harvested for staining of the human marker LDH (light grey) and the viral transgene td-Tomato (black) to show co-localization.

FIGS. 7A and 7B depict transduction of AAV9 in humanized TIRF and TIRFA mice. Humanized TIRF (transgene free Il2rg^{-/-}/Rag2^{-/-}/Fah^{-/-}/Aavr^{+/+}) (FIG. 7A) and humanized TIRFA (transgene free Il2rg^{-/-}/Rag2^{-/-}/Fah^{-/-}/Aavr^{-/-}) (FIG. 7B) mice were injected with 1×10¹² GC/mouse of AAV9 carrying a td-Tomato expression cassette. Mice were euthanized after 72 hours, and livers harvested for staining of the human marker LDH (light grey) and the viral transgene td-Tomato (black) to show co-localization.

FIGS. 8A and 8B depict quantification of AAV transduction efficiency of human hepatocytes in humanized TIRFA mice Humanized TIRF (transgene free Il2rg^{-/-}/Rag2^{-/-}/Fah^{-/-}/Aavr^{+/+}) and humanized TIRFA (transgene free Il2rg^{-/-}/Rag2^{-/-}/Fah^{-/-}/Aavr^{-/-}) mice were injected with 1×10¹² GC/mouse of AAV8 (FIG. 8A) or AAV9 (FIG. 8B) carrying a td-Tomato expression cassette. Mice were euthanized after 72 hours, and livers harvested for staining of the human marker LDH and the viral transgene (td-Tomato). Depicted is the quantification of colocalization of td-Tomato and LDH, e.g. AAV transduction of human hepatocytes in TIRF and TIRFA mice. Three mice with 3-5 liver sections each were used for this quantification.

FIG. 9 is a schematic showing the experimental setup for the teratoma assay described in Example 5.

FIG. 10 depicts fluorescence of freshly harvested liver and teratoma. Humanized TIRFA and non-humanized TIRFA mice were injected subcutaneously with ˜1×10⁷ induced pluripotent stem (iPS) cells and after development of teratoma (˜3 months post injection) intravenously injected with 1×10¹² GC/mouse of AAV9 carrying an expression cassette of GFP. 72 hours after AAV injection mouse was euthanized. Liver and teratoma were exposed to low wavelength lamp to demonstrate fluorescence of tissue. Note, patchy fluorescence of TIRFA mouse liver is originating from human liver areas.

FIG. 11 depicts AAV9 transduction efficiency of human cells in teratoma of TIRFA mouse without human liver. TIRFA mouse was injected subcutaneously with ˜1×10⁷ induced pluripotent stem (iPS) cells and after development of teratoma (3 months post injection) intravenously injected with 1×10¹² GC/mouse of AAV9 carrying an expression cassette of GFP. 72 hours after AAV injection mouse was euthanized and teratoma analyzed by H&E staining (11A) and GFP immunostaining (11B). Shown are serial sections of the teratoma indicating differential transduction efficiencies of different human tissues in the teratoma.

FIG. 12 depicts AAV9 transduction efficiency of human intestinal cells in teratoma of TIRFA mouse without human liver. The teratoma is the same as shown in FIG. 11, but shown here is a selection of endodermal tissue, e.g. intestinal tissue in teratoma transduced with AAV9-GFP.

FIG. 13 depicts AAV9 transduction efficiency of human mesoderm in teratoma of TIRFA mouse without human liver. The teratoma is the same as shown in FIG. 11, but shown here is a selection of mesodermal tissue transduced by AAV9-GFP.

FIGS. 14A-14D depict AAV9 transduction efficiencies of human teratoma of TIRFA mouse with human liver. Human liver TIRFA mouse was injected subcutaneously with ˜10E7 induced pluripotent stem (iPS) cells and after development of teratoma (3 months post injection) intravenously injected with 1×10¹² GC/mouse of AAV9 carrying an expression cassette of GFP. 72 hours after AAV injection mouse was euthanized and analyzed by H&E staining (14A and 14C) and GFP immunostaining (14B and 14D). Shown are serial sections of the teratoma indicating differential transduction efficiencies of human teratoma tissue. The arrow (14C and 14D) shows non-transduced smooth muscle tissue and the arrowhead (14C and 14D) transduced glandular structures. Box in 14A is magnified area of teratoma in 14C and box in 14B is magnified area in 14D.

### DETAILED DESCRIPTION OF THE INVENTION

The present disclosure provides a chimeric non-human animal comprising human hepatocytes, wherein the chimeric non-human animal comprises: a) a T-, B- and/or NK cell deficiency or functional impairment that allows repopulating with human hepatocytes to establish human chimerism in the liver of the non-human animal; and b) a deletion or mutation of Adeno-associated virus receptor (AAVR) resulting in deficiency or functional impairment of the non-human animal AAVR.

The present disclosure provides a chimeric non-human animal comprising human hepatocytes, wherein the chimeric non-human animal is a IL-2Rg−/−/Rag 2−/− chimeric non-human animal, and wherein the chimeric non-human animal comprises a deletion or mutation of AAVR resulting in deficiency or functional impairment of the non-human animal AAVR. In some embodiments, the chimeric non-human animal further comprises Fah−/−.

In some embodiments of the chimeric non-human animals of the disclosure, the chimeric non-human animal is a IL-2rg−/−/Rag 2−/−/Fah−/− non-human animal. In some embodiments of the chimeric non-human animals of the disclosure, the human hepatocytes account for at least 5%; at least 10%; at least 20%; at least 30%; at least 40%; at least 50%; at least 70%; at least 80%; at least 90%; at least 95% or at least 99% of all the hepatocytes in the liver of the chimeric non-human animal are human hepatocytes. In some embodiments of the chimeric non-human animals of the disclosure, the human hepatocytes account for at least 70% (e.g., 70%, 80%, 90%, 95%, or 97%) of all the hepatocytes in the liver of the chimeric non-human animal.

In some embodiments of the chimeric non-human animals of the disclosure, upon infection with an AAV vector less than 50%, less than 40%, less than 30%, less than 20%, less than 10% or less than 5% of the endogenous hepatocytes of the non-human chimeric animal are transduced with the AAV vector.

In some embodiments of the chimeric non-human animals of the disclosure, upon infection with an AAV vector at least 10% c, at least 20%, at least 30%, at least 50% c, at least 70%, at least 80% or at least 90% of the human hepatocytes are transduced with the AAV vector. In some embodiments of the chimeric non-human animals of the disclosure, upon infection with an AAV vector at least 50% (e.g., 50%, 60%, 70%, 80%, 90%, 95%, 99% or 100%) of the human hepatocytes are transduced with the AAV vector.

Adeno-associated virus (AAV), a member of the Parvovirus family, is a small nonenveloped, icosahedral virus with a single-stranded linear DNA genome of 4.7 kilobases (kb) to 6 kb. The life cycle of AAV comprises a latent phase at which AAV genomes, after infection, are integrated into host genomes and an infectious phase in which, following either adenovirus or herpes simplex virus infection, the integrated AAV genomes are subsequently rescued, replicated, and packaged into infectious viruses. The properties of non-pathogenicity, broad host range of infectivity, including non-dividing cells, and integration make AAV an attractive delivery vehicle.

In some embodiments of the chimeric non-human animals of the disclosure, the AAV is any one of the AAV serotypes 1, 2, 3, 4, 5, 6, 7, 8, 9, 10 and 11. In some embodiments of the chimeric non-human animals of the disclosure, the AAV is AAV serotype 8. In some embodiments of the chimeric non-human animals of the disclosure, the AAV is AAV serotype 9. In some embodiments of the chimeric non-human animals of the disclosure, the AAV is a AAV serotype that specifically infects or transduces the liver of a subject. In some embodiments of the chimeric non-human animals of the disclosure, the AAV is a AAV serotype that specifically infects or transduces hepatocytes. In some embodiments of the chimeric non-human animals of the disclosure, the AAV is a hybrid of two or more AAV serotypes. In some embodiments of the chimeric non-human animals of the disclosure, the AAV is a variant selected from any one of 6.2, 2, rh64R1, rh10, 8, 9 and AAV9-PHP.B. In some embodiments of the chimeric non-human animals of the disclosure, the AAV vector encodes a detectable molecule. In some embodiments of the chimeric non-human animals of the disclosure, the detectable molecule is a fluorescent protein, an enzyme, or a peptide. In some embodiments of the chimeric non-human animals of the disclosure, the detectable molecule is GFP, RFP, YFP, CFP, dTomato, mCherry or LacZ (R-galactosidase). In some embodiments of the chimeric non-human animals of the disclosure, the AAV vector comprises an inducible promoter or a constitutive promoter. In some embodiments of the chimeric non-human animals of the disclosure, the AAV vector comprises a tissue specific promoter. In some embodiments of the chimeric non-human animals of the disclosure, the inducible promoter is a tetracycline-inducible promoter or a CMV promoter. In some embodiments of the chimeric non-human animals of the disclosure, the AAV vector encodes one or more heterologous proteins. In some embodiments of the chimeric non-human animals of the disclosure, the one or more heterologous proteins can be selected from any one an immunogenic protein or peptide, a therapeutic protein, a regulatory protein or a marker/detectable protein.

In some embodiments of the chimeric non-human animals of the disclosure, the chimeric non-human animal can be any one of a primate, bird, mouse, rat, fowl, dog, cat, cow, horse, goat, camel, sheep and pig. In some embodiments of the chimeric non-human animals of the disclosure, the chimeric non-human animal is a mouse.

The present disclosure also provides a method for preparing a chimeric non-human animal comprising human hepatocytes, the method comprising: (a) providing a T-, B- and/or NK cell deficient or impaired in function non-human animal, that allows re-populating with human hepatocytes to establish human chimerism in the liver of the non-human animal, wherein the non-human animal comprises a deletion or mutation of AAVR resulting in a non-functional non-human animal AAVR; and (b) transplanting human hepatocytes into the non-human animal. In some embodiments of the method for preparing a chimeric non-human animal comprising human hepatocytes of the present disclosure, step (b) further comprises applying a selection pressure.

The present disclosure also provides a method for preparing a chimeric non-human animal comprising human hepatocytes, the method comprising: (a) providing a IL-2Rg−/−/Rag 2−/− non-human animal, wherein the non-human animal comprises a deletion or mutation of Adeno-associated virus receptor (AAVR) resulting in a non-functional non-human animal AAVR; and (b) transplanting human hepatocytes into the non-human animal. In some embodiments of the methods of this disclosure, the non-human animal further comprises a Fah−/−. In some embodiments of the method for preparing a chimeric non-human animal comprising human hepatocytes of the present disclosure, step (b) further comprises applying a selection pressure.

In some embodiments of the methods of this disclosure, applying a selection pressure comprises not providing nitisinone (NTBC) to the non-human animal of step (b). In some embodiments of the methods of this disclosure, the method can further comprise removal of the selection pressure following step (c) of the methods disclosed herein. The removal of the selection pressure can comprise providing nitisinone (NTBC) to the chimeric non-human animal following step (c) of the methods disclosed herein. In some embodiments of the methods of this disclosure, the chimeric non-human animal does not comprise a transgene. In some embodiments of the methods of this disclosure, the transgene is an antibiotic resistance cassette. The present disclosure also provides a chimeric non-human animal produced by the methods disclosed herein.

In some embodiments of the methods of preparing of this disclosure, the chimeric non-human animal is a IL-2Rg−/−/Rag 2−/−/Fah−/− non-human animal. In some embodiments of the methods of preparing of this disclosure, the chimeric non-human animal is a IL-2Rg−/−/Rag 2−/−/Fah−/− non-human animal, and wherein the chimeric non-human animal does not comprise an antibiotic selection gene cassette.

In some embodiments of the methods of preparing of the disclosure, at least 5%, at least 10%, at least 20%, at least 30%, at least 40%, at least 50%, at least 70%, at least 80%, at least 90%, at least 95% or at least 97% of all the hepatocytes in the liver of the chimeric non-human animal are human hepatocytes. In some embodiments of the methods of preparing of this disclosure, the human hepatocytes account for at least 70% (e.g., 70%, 80%, 90%, 95%, or 97%) of all the hepatocytes in the liver of the chimeric non-human animal are human hepatocytes.

In some embodiments of the methods of preparing of this disclosure, after infection with an AAV vector less than 50%, less than 40%, less than 30%, less than 20%, less than 10% or less than 5% of the endogenous hepatocytes of the non-human chimeric animal are transduced with the AAV vector.

In some embodiments of the methods of preparing of this disclosure, after infection with an AAV vector at least 10%, at least 20%, at least 30%, at least 50%, at least 70%, at least 80% or at least 90% of the human hepatocytes are transduced with the AAV vector. In some embodiments of the methods of preparing of this disclosure, after infection with an AAV vector at least 50% (e.g., 50%, 60%, 70%, 80%, 90%, 95%, 99% or 100%) of the human hepatocytes are transduced with the AAV vector.

In some embodiments of the methods of preparing of this disclosure, the AAV is any one of the AAV serotypes 1, 2, 3, 4, 5, 6, 7, 8, 9, 10 and 11. In some embodiments of the methods of preparing of this disclosure, the AAV is AAV serotype 8. In some embodiments of the methods of preparing of this disclosure, the AAV is AAV serotype 9. In some embodiments of the methods of preparing of this disclosure, the AAV is a AAV serotype that specifically infects and/or transduces the liver of a subject. In some embodiments of the methods of preparing of this disclosure, the AAV is a AAV serotype that specifically infects and/or transduces hepatocytes. In some embodiments of the methods of preparing of this disclosure, the AAV is a hybrid of two or more AAV serotypes. In some embodiments of the methods of preparing of this disclosure, the AAV is a variant selected from any one of 6.2, 2, rh64R1, rh10, 8, 9 and AAV9-PHP.B. In some embodiments of the methods of preparing of this disclosure, the AAV vector encodes a detectable molecule. In some embodiments of the methods of preparing of this disclosure, the detectable molecule is a fluorescent protein, an enzyme, or a peptide. In some embodiments of the methods of preparing of this disclosure, the detectable marker is GFP, RFP, YFP, CFP, dTomato, mCherry or LacZ (β-galactosidase). In some embodiments of the methods of preparing of this disclosure, the AAV vector comprises an inducible promoter or a constitutive promoter. In some embodiments of the methods of preparing of this disclosure, the AAV vector comprises a tissue-specific promoter. In some embodiments of the methods of preparing of this disclosure, the inducible promoter a tetracycline-inducible promoter or a CMV promoter. In some embodiments of the methods of preparing of this disclosure, the AAV vector encodes one or more heterologous proteins. In some embodiments of the methods of preparing of this disclosure, the one or more heterologous proteins can be selected from any one an immunogenic protein or peptide, a therapeutic protein, a regulatory protein or a marker/detectable protein. In some embodiments of the methods of preparing of this disclosure, the AAV vector comprises a unique nucleic acid sequence in the genome of the vector, wherein the unique nucleic acid sequence can be transcribed into a detectable RNA sequence or a barcode RNA sequence. The detectable RNA sequence or barcode RNA sequence of an AAV vector is different from the detectable RNA sequence or a barcode RNA sequence of any other vector used in the methods of the disclosure. The AAV vector can comprise any unique nucleic acid sequence in the genome of an AAV vector or detectable RNA sequence or barcode RNA sequence of an AAV vector known in the art, for example in Adachi K et al., Molecular Therapy Volume 22, Supplement 1, May 2014; Adachi K et al., Nature Communications volume 5, Article number: 3075 (2014); Pekrun K et al., JCI Insight. 2019; 4(22):e131610; US20190135871A1; and WO2020160508A1.

In some embodiments of the methods of preparing of this disclosure, the AAVR of the non-human animal can be deleted by CRISPR/Cas9 mediated deletion of the non-human animal AAVR, or knockdown with exogenous agents or floxed allele of the non-human animal AAVR gene with a first dose of a virus that encodes Cre recombinase or a Cre transgenic non-human animal. In some embodiments of the methods of preparing of this disclosure, the AAVR of the non-human animal can be deleted by one or more RNA interference (RNAi) targeting the AAVR mRNA. In some embodiments of the methods of preparing of this disclosure, the variant AAVR can comprise one or more altered amino acids relative to the amino acid sequence of a wild type AAVR, resulting in reduced in surface expression level or cell surface trafficking, compared to a wild type AAVR. In some embodiments of the methods of preparing of this disclosure, the variant AAVR comprises one or more altered amino acids and/or a deletion in the signaling peptide, one or more of the polycystic kidney disease-like (PKD) domains, the motif at the N terminus with eight cysteines (MANEC) domain, a transmembrane domain and/or the cytosolic tail, relative to the amino acid sequence of a wild type AAVR.

In some embodiments of the methods of preparing of this disclosure, the chimeric non-human animal can be any one of a primate, bird, mouse, rat, fowl, dog, cat, cow, horse, goat, camel, sheep and pig. In some embodiments of the methods of preparing of this disclosure, the chimeric non-human animal is a mouse.

The present disclosure also provides a method of determining transduction efficiency of an AAV vector in human hepatocytes, wherein the method comprises: (a) providing a chimeric non-human animal prepared by any one of the methods of the disclosure; (b) infecting the non-human animal of (a) with an amount of the AAV vector; (c) maintaining the infected non-human animal of (b) for an amount of time; and (d) determining the level of transduction of the AAV vector into the human hepatocytes and the hepatocytes of the AAVR KO non-human animal (non-human animal hepatocytes).

In some embodiments of the method of determining transduction efficiency of the disclosure, in step (d) the level of transduction of the AAV vector into the human hepatocytes or the non-human animal hepatocytes, is measured as: (i) percentage of the AAV vector-transduced human hepatocytes or percentage of AAV vector-transduced non-human animal hepatocytes, respectively, in the non-human animal; or (ii) percentage of the total amount of the AAV vector-transduced into the human hepatocytes or the non-human animal hepatocytes, respectively, in the non-human animal.

In some embodiments of the method of determining transduction efficiency of the disclosure, the method further comprises: (f) selecting the AAV vector as efficient in transducing human hepatocytes if (i) less than a pre-determined percentage of the total amount of the AAV vector is transduced into the non-human animal hepatocytes; (ii) at least a pre-determined percentage of the total amount of AAV vectors is transduced into the human hepatocytes; (iii) percentage of AAV vector-transduced non-human animal hepatocytes is less than a pre-determined value; and/or (iv) percentage of AAV vector-transduced human animal hepatocytes is more than a pre-determined value.

The present disclosure also provides a method of determining transduction efficiency of two or more non-identical AAV vector(s) in human hepatocytes, wherein the method comprises: (a) providing two or more chimeric AAVR KO non-human animal generated by any one of the methods of the disclosure; (b) infecting each of the non-human animals of (a) with an amount of the two or more non-identical AAV vectors, wherein one non-human animal is infected with one AAV vector and the other animal is infected with the other AAV vector; (c) maintaining the infected non-human animals of (b) for an amount of time; and (d) determining the level of transduction of the AAV vector into the human hepatocytes and the hepatocytes of the AAVR KO non-human animal (non-human animal hepatocytes) in each of the two or more non-human animals of (c). In some embodiments of the methods of this disclosure, the method further comprises removing the selection pressure following step (b). In some embodiments of the methods of this disclosure, the method further comprises: (e) comparing the level of transduction of each of the two or more AAV vectors into the non-human hepatocytes determined in (d); and/or (t) comparing the level of transduction of each of the two or more AAV vectors into the human hepatocytes determined in (d). In some embodiments of the methods of this disclosure, the method further comprises: (g) selecting one or more AAV vector(s) as efficient in transducing human hepatocytes if: (i) less than a pre-determined percentage of the total amount of the AAV vector is transduced into the non-human animal hepatocytes; (ii) at least a pre-determined percentage of the total amount of AAV vectors is transduced into the human hepatocytes; (iii) percentage of AAV vector-transduced non-human animal hepatocytes is less than a pre-determined value; and/or (iv) percentage of AAV vector-transduced human animal hepatocytes is more than a pre-determined value.

In some embodiments of the methods of determining transduction efficiency of AAV vector of this disclosure, in (i) the predetermined percentage of the total amount of AAV vectors transduced into the non-human animal hepatocytes is ≤100%, ≤90%, ≤80%, ≤70%, ≤50%, ≤20%, ≤10%, or ≤5%. In some embodiments of the methods of determining transduction efficiency of AAV vector of this disclosure, the percentage of the total amount of AAV vectors transduced into the non-human animal hepatocytes is 70% to 90%. In some embodiments of the methods of determining transduction efficiency of AAV vector of this disclosure, the percentage of the total amount of AAV vectors transduced into the non-human animal hepatocytes is 50% to 70%. In some embodiments of the methods of determining transduction efficiency of AAV vector of this disclosure, the percentage of the total amount of AAV vectors transduced into the non-human animal hepatocytes is 20% to 50%. In some embodiments of the methods of determining transduction efficiency of AAV vector of this disclosure, the percentage of the total amount of AAV vectors transduced into the non-human animal hepatocytes is 10% to 20%. In some embodiments of the methods of determining transduction efficiency of AAV vector of this disclosure, the percentage of the total amount of AAV vectors transduced into the non-human animal hepatocytes is 5% to 10%. In some embodiments of the methods of determining transduction efficiency of AAV vector of this disclosure, the percentage of the total amount of AAV vectors transduced into the non-human animal hepatocytes is 0% to 5%.

In some embodiments of the method of determining transduction efficiency of AAV vector of this disclosure, wherein in (ii) the predetermined percentage of the total amount of AAV vectors transduced into the human hepatocytes is ≥99%, ≥95%, ≥90%, ≥80%, ≤70%, ≥50%, ≥20% or ≤10%. In some embodiments of the methods of determining transduction efficiency of AAV vector of this disclosure, the percentage of the total amount of AAV vectors transduced into the human hepatocytes is 95% to 100%. In some embodiments of the methods of determining transduction efficiency of AAV vector of this disclosure, the percentage of the total amount of AAV vectors transduced into the human hepatocytes is 90% to 95%. In some embodiments of the method of determining transduction efficiency of AAV vector of this disclosure, the percentage of the total amount of AAV vectors transduced into the human hepatocytes is 80% to 90%. In some embodiments of the methods of determining transduction efficiency of AAV vector of this disclosure, the percentage of the total amount of AAV vectors transduced into the human hepatocytes is 70% to 80%. In some embodiments of the methods of determining transduction efficiency of AAV vector of this disclosure, the percentage of the total amount of AAV vectors transduced into the human hepatocytes is 50% to 70%. In some embodiments of the methods of determining transduction efficiency of AAV vector of this disclosure, the percentage of the total amount of AAV vectors transduced into the human hepatocytes is 20% to 50%. In some embodiments of the methods of determining transduction efficiency of AAV vector of this disclosure, the percentage of the total amount of AAV vectors transduced into the human hepatocytes is 10% to 20%.

In some embodiments of the methods of determining transduction efficiency of AAV vector of this disclosure, the percentage of the total amount of AAV vectors transduced into the non-human animal hepatocytes is 0% to 5%, and the percentage of the total amount of AAV vectors transduced into the human hepatocytes is 95% to 100%. In some embodiments of the methods of determining transduction efficiency of AAV vector of this disclosure, the percentage of the total amount of AAV vectors transduced into the non-human animal hepatocytes is 5% to 10%, and the percentage of the total amount of AAV vectors transduced into the human hepatocytes is 90% to 95%. In some embodiments of the methods of determining transduction efficiency of AAV vector of this disclosure, the percentage of the total amount of AAV vectors transduced into the non-human animal hepatocytes is 10% to 20%, and the percentage of the total amount of AAV vectors transduced into the human hepatocytes is 80% to 90%. In some embodiments of the methods of determining transduction efficiency of AAV vector of this disclosure, the percentage of the total amount of AAV vectors transduced into the non-human animal hepatocytes is 20% to 50%, and the percentage of the total amount of AAV vectors transduced into the human hepatocytes is 50% to 80%. In some embodiments of the methods of determining transduction efficiency of AAV vector of this disclosure, the percentage of the total amount of AAV vectors transduced into the non-human animal hepatocytes is 50% to 70%, and the percentage of the total amount of AAV vectors transduced into the human hepatocytes is 20% to 50%. In some embodiments of the methods of determining transduction efficiency of AAV vector of this disclosure, the percentage of the total amount of AAV vectors transduced into the non-human animal hepatocytes is 70% to 80%, and the percentage of the total amount of AAV vectors transduced into the human hepatocytes is 10% to 20%. In some embodiments of the methods of determining transduction efficiency of AAV vector of this disclosure, the percentage of the total amount of AAV vectors transduced into the non-human animal hepatocytes is 80% to 90%, and the percentage of the total amount of AAV vectors transduced into the human hepatocytes is 10% to 20%.

The percentages of the total amount of AAV vectors transduced into the non-human animal hepatocytes and non-human hepatocytes, depend on the specific AAV serotype used to infect the chimeric non-human animal of the present disclosure. In some embodiments, the transduction of the AAV vector into a non-human hepatocyte, can be mediated only by AAVR. In some embodiments, the transduction of the AAV vector into a non-human hepatocyte, can be mediated only by one or more receptors other than AAVR (non-AAVR receptors). In some embodiments, the transduction of the AAV vector into a non-human hepatocyte, can be mediated by both AAVR and one or more receptors other than AAVR (non-AAVR receptors).

The percentage of the total amount of AAV vectors transduced into the non-human animal hepatocytes can be determined by any of the methods known in the art, including by harvesting the liver of the chimeric non-human animal infected with the AAV vector, titrating the amount of the AAV vector recovered from the human hepatocytes or the non-human hepatocytes or both isolated from the liver, and comparing or determining a ratio of the titrated amount of the recovered AAV vector to the total amount of AAV vector used to infect the animal. In some aspect, the titrated amount of the recovered AAV vector from the liver or the ratio of the titrated amount of the recovered AAV vector to the total amount of AAV vector, can be correlated with the percentage of endogenous/non-human hepatocytes of the chimeric non-human animal transduced with the AAV.

In some embodiments, the percentage of the total amount of AAV vectors transduced into the non-human animal hepatocytes can be determined by generating a standard curve by comparing/plotting the mean detectable signal intensity of the total number of transduced mouse cells/hepatocytes or total number of transduced mouse cells/hepatocytes, in a control non-human animal with respect to/against increasing amounts of an AAV vector used to transduce the non-human animal. The control non-human animal as described herein can be a wild type or IL-2Rg−/−/Rag 2−/− non-human animal or a IL-2Rg−/−/Rag 2−/−/Fah−/− non-human animal or a non-human animal with T-, B- and/or NK cell deficiency or functional impairment, without a deletion or mutation of Adeno-associated virus receptor (AAVR). The percentage of the total amount of AAV vectors transduced into the non-human animal hepatocytes or the human hepatocytes or both of the chimeric non-human animal of the methods of this disclosure can be determined based on the standard curve described herein. In some embodiments, it is assumed that all or at least 99% of the total amount of AAV vectors used to infect a control non-human animal are transduced into the non-human animal hepatocytes.

In some embodiments of the methods of determining transduction efficiency of AAV vector of this disclosure, in (iii) the pre-determined value of the percentage of AAV vector-transduced non-human animal hepatocytes is ≤50%, ≤40%, ≤30%, ≤20%, ≤10% or ≤5%.

In some embodiments of the methods of determining transduction efficiency of AAV vector of this disclosure, the percentage of AAV vector-transduced-non-human animal hepatocytes is 40% to 50%. In some embodiments of the methods of determining transduction efficiency of AAV vector of this disclosure, the percentage of AAV vector-transduced non-human animal hepatocytes is 30% to 40%. In some embodiments of the methods of determining transduction efficiency of AAV vector of this disclosure, the percentage of AAV vector-transduced non-human animal hepatocytes is 20% to 30%. In some embodiments of the methods of determining transduction efficiency of AAV vector of this disclosure, the percentage of AAV vector-transduced non-human animal hepatocytes is 10% to 20%. In some embodiments of the methods of determining transduction efficiency of AAV vector of this disclosure, the percentage of AAV vector-transduced non-human animal hepatocytes is 5% to 10%. In some embodiments of the methods of determining transduction efficiency of AAV vector of this disclosure, the percentage of AAV vector-transduced non-human animal hepatocytes is 0% to 5%.

In some embodiments of the methods of determining transduction efficiency of AAV vector of this disclosure, in (iv) the pre-determined value of the percentage of AAV vector-transduced human animal hepatocytes is ≥10%, ≥20%, ≥30%, ≥50%, ≥70%, ≥80% or ≥90%.

In some embodiments of the methods of determining transduction efficiency of AAV vector of this disclosure, the percentage of AAV vector-transduced human hepatocytes is 90% to 97%. In some embodiments of the methods of determining transduction efficiency of AAV vector of this disclosure, the percentage of AAV vector-transduced human hepatocytes is 80% to 90%. In some embodiments of the methods of determining transduction efficiency of AAV vector of this disclosure, the percentage of AAV vector-transduced human animal hepatocytes is 70% to 80%. In some embodiments of the methods of determining transduction efficiency of AAV vector of this disclosure, the percentage of AAV vector-transduced human hepatocytes is 50% to 70%. In some embodiments of the methods of determining transduction efficiency of AAV vector of this disclosure, the percentage of AAV vector-transduced human hepatocytes is 20% to 50%. In some embodiments of the methods of determining transduction efficiency of AAV vector of this disclosure, the percentage of AAV vector-transduced human hepatocytes is 10% to 20%.

In some embodiments of the methods of determining transduction efficiency of AAV vector of this disclosure, the percentage of AAV vector-transduced non-human animal hepatocytes can be 40% to 50%, 30% to 40%, 20% to 30%, 10% to 20%, 5% to 10% or 0% to 5%, and the percentage of AAV vector-transduced human animal hepatocytes can be 10% to 20%, 20% to 30%, 30% to 40%, 40% to 50%, 50% to 70%, 70% to 80%, 80% to 90% or 90% to 100%.

The percentages of AAV vector-transduced non-human animal hepatocytes and non-human hepatocytes, depend on the specific AAV serotype used to infect the chimeric non-human animal of the present disclosure. In some embodiments, the transduction of the AAV vector into a human or a non-human hepatocyte, can be mediated only by AAVR. In some embodiments, the transduction of the AAV vector into a human or a non-human hepatocyte, can be mediated only by one or more receptors other than AAVR (non-AAVR receptors). In some embodiments, the transduction of the AAV vector into a human or a non-human hepatocyte can be mediated by both AAVR and one or more receptors other than AAVR (non-AAVR receptors). The transduction of an AAV into a human or a non-human hepatocyte can be mediated by either an AAVR or a non-AAVR receptor, depending on the specific serotype or variant of a serotype of an AAV. The transduction of an AAV into a human or a non-human hepatocyte can be mediated by either an AAVR or a non-AAVR receptor, depending on the specific capsid proteins of an AAV.

In some embodiments method of determining transduction efficiency of AAV vector of this disclosure, the amount of AAV vector in the infection is 1×10¹ to 1×10¹⁵ viral genomes per non-human animal. In some embodiments method of determining transduction efficiency of AAV vector in the infection of this disclosure, the amount of AAV vector in the infection is 1×10² to 1×10¹² viral genomes per non-human animal. In some embodiments method of determining transduction efficiency of AAV vector of this disclosure, the amount of AAV vector in the infection is 1×10⁴ to 1×10¹⁰ viral genomes per non-human animal. In some embodiments of the methods of determining transduction efficiency of AAV vector of this disclosure, the amount of AAV vector in the infection is 1×10⁶ to 1×10⁸ viral genomes per non-human animal. In some embodiments methods of determining transduction efficiency of AAV vector of this disclosure, the amount of AAV vector in the infection is 1×10¹ to 1×10³ viral genomes per non-human animal. In some embodiments methods of determining transduction efficiency of AAV vector of this disclosure, the amount of AAV vector in the infection is 1×10² to 1×10⁵ viral genomes per non-human animal. In some embodiments of the methods of determining transduction efficiency of AAV vector of this disclosure, the amount of AAV vector in the infection is 1×10⁵ to 1×10⁸ viral genomes per non-human animal. In some embodiments of the methods of determining transduction efficiency of AAV vector of this disclosure, the amount of AAV vector in the infection is 1×10⁸ to 1×10¹² viral genomes per non-human animal. In some embodiments of the methods of determining transduction efficiency of AAV vector of this disclosure, the amount of AAV vector in the infection is 1×10¹² to 1×10¹⁵ viral genomes per non-human animal.

In some embodiments of the methods of determining transduction efficiency of AAV vector of this disclosure, the maintaining of the infected non-human animal is done for at least 1 day. In some embodiments of the methods of determining transduction efficiency of AAV vector of this disclosure, the maintaining of the infected non-human animal is done for at least 1 week. In some embodiments of the methods of determining transduction efficiency of AAV vector of this disclosure, the maintaining of the infected non-human animal is done for at least 2 weeks. In some embodiments of the methods of determining transduction efficiency of AAV vector of this disclosure, the maintaining of the infected non-human animal is done for at least 4 weeks. In some embodiments of the methods of determining transduction efficiency of AAV vector of this disclosure, the maintaining of the infected non-human animal is done for 1 day to 5 days. In some embodiments of the methods of determining transduction efficiency of AAV vector of this disclosure, the maintaining of the infected non-human animal is done for 5 days to 10 days. In some embodiments of the methods of determining transduction efficiency of AAV vector of this disclosure, the maintaining of the infected non-human animal is done for 10 days to 15 days. In some embodiments of the methods of determining transduction efficiency of AAV vector of this disclosure, the maintaining of the infected non-human animal is done for 15 days to 20 days. In some embodiments of the methods of determining transduction efficiency of AAV vector of this disclosure, the maintaining of the infected non-human animal is done for 20 days to 25 days. In some embodiments of the methods of determining transduction efficiency of AAV vector of this disclosure, the maintaining of the infected non-human animal is done for 25 days to 30 days.

In some embodiments of the methods of determining transduction efficiency of AAV vector of this disclosure, the AAV is any one of the AAV serotypes 1, 2, 3, 4, 5, 6, 7, 8, 9, 10 and 11. In some embodiments of the methods of determining transduction efficiency of AAV vector of this disclosure, the AAV is AAV serotype 8. In some embodiments of the methods of determining transduction efficiency of AAV vector of this disclosure, the AAV is AAV serotype 9. In some embodiments of the methods of determining transduction efficiency of AAV vector of this disclosure, the AAV is a AAV serotype that specifically infects or transduces the liver of a subject. In some embodiments of the methods of determining transduction efficiency of AAV vector of this disclosure, the AAV is a AAV serotype that specifically infects or transduces hepatocytes. In some embodiments of the methods of determining transduction efficiency of AAV vector of this disclosure, the AAV is a hybrid of two or more AAV serotypes. In some embodiments of the methods of determining transduction efficiency of AAV vector of this disclosure, the AAV is a variant selected from any one of 6.2, 2, rh64R1, rh10, 8, 9 and AAV9-PHP.B. In some embodiments of the methods of determining transduction efficiency of AAV vector of this disclosure, the AAV vector encodes a detectable marker. In some embodiments of the methods of determining transduction efficiency of AAV vector of this disclosure, the detectable marker is a fluorescent protein, an enzyme, or a peptide. In some embodiments of the methods of determining transduction efficiency of AAV vector of this disclosure, the detectable marker is GFP, RFP, YFP, CFP, dTomato, mCherry or LacZ (β-galactosidase). In some embodiments of the methods of determining transduction efficiency of AAV vector of this disclosure, the AAV vector comprises an inducible promoter or a constitutive promoter. In some embodiments of the methods of determining transduction efficiency of AAV vector of this disclosure, the AAV vector comprises a tissue specific promoter. In some embodiments of the methods of determining transduction efficiency of AAV vector of this disclosure, the inducible promoter is a tetracycline-inducible promoter or a CMV promoter. In some embodiments of the methods of determining transduction efficiency of AAV vector of this disclosure, the AAV vector encodes one or more heterologous proteins. In some embodiments of the methods of determining transduction efficiency of AAV vector of this disclosure, the one or more heterologous proteins can be selected from any one an immunogenic protein or peptide, a therapeutic protein, a regulatory protein or a marker/detectable protein.

The present disclosure also provides a method of determining the efficiency of a systemic AAV vector-mediated gene therapy, wherein the method comprises: (a) providing a chimeric AAVR KO non-human animal prepared by any of the methods of the disclosure; (b) infecting the non-human animal of (a) with an amount of an AAV vector, at least a first time; (c) maintaining the infected non-human animal of (b) for an amount of time; and (d) determining the level of transduction of the AAV vector into the human hepatocytes of the AAVR KO non-human animal. In some embodiment of the method of determining the efficiency of a systemic AAV vector-mediated gene therapy of this disclosure, the method comprises: (e) infecting the non-human animal with an amount of an AAV vector, a second time; (t) maintaining the infected non-human animal of (e) for an amount of time; and (g) determining the level of transduction of the AAV vector into the human hepatocytes of the AAVR KO non-human animal. In some embodiments of the method of determining the efficiency of a systemic AAV-vector-mediated gene therapy of this disclosure, the method further comprises: (i) comparing the level of transduction of the AAV vector into the human hepatocytes between the first and the second infection.

In some embodiments of the method of determining the efficiency of a systemic AAV vector-mediated gene therapy of this disclosure, the amounts of AAV vector in the first and the second infection are same. In some embodiments of the method of determining the efficiency of a systemic AAV vector-mediated gene therapy of this disclosure, the amounts of AAV vector in the first and the second infection are different. In some embodiments of the method of determining the efficiency of a systemic AAV vector-mediated gene therapy of this disclosure, the amount of AAV vector in the first is higher than the amount of AAV vector in the second infection. In some embodiments of the method of determining the efficiency of a systemic AAV vector-mediated gene therapy of this disclosure, the amount of AAV vector in the first is lower than the amount of AAV vector in the second infection.

In some embodiments of the method of determining the efficiency of a systemic AAV vector-mediated gene therapy of the disclosure, the amount of AAV vector in the first/and or second infection is 1×10¹ to 1×10⁵ viral genomes per non-human animal. In some embodiments of the method of determining the efficiency of a systemic AAV vector-mediated gene therapy of this disclosure, the amount of AAV vector in the first/and or second infection is 1×10² to 1×10¹² viral genomes per non-human animal. In some embodiments of the method of determining transduction efficiency of AAV vector of this disclosure, the amount of AAV vector in the first/and or second infection is 1×10⁴ to 1×10¹⁰ viral genomes per non-human animal. In some embodiments of the method of determining transduction efficiency of AAV vector of this disclosure, the amount of AAV vector in the first/and or second infection is 1×10⁶ to 1×10⁸ viral genomes per non-human animal. In some embodiments method of determining the efficiency of a systemic AAV vector-mediated gene therapy of this disclosure, the amount of AAV vector in the first/and or second infection is 1×10¹ to 1×10³ viral genomes per non-human animal. In some embodiments of the method of determining the efficiency of a systemic AAV vector-mediated gene therapy of this disclosure, the amount of AAV vector in the first/and or second infection is 1×10² to 1×10⁵ viral genomes per non-human animal. In some embodiments of the method of determining the efficiency of a systemic AAV vector-mediated gene therapy of this disclosure, the amount of AAV vector in the first/and or second infection is 1×10⁵ to 1×10⁸ viral genomes per non-human animal. In some embodiments of the method of determining the efficiency of a systemic AAV vector-mediated gene therapy of this disclosure, the amount of AAV vector in the first/and or second infection is 1×10⁸ to 1×10¹² viral genomes per non-human animal. In some embodiments of the method of determining the efficiency of a systemic AAV vector-mediated gene therapy of this disclosure, the amount of AAV vector in the first/and or second infection is 1×10¹² to 1×10¹⁵ viral genomes per non-human animal.

In some embodiments of the method of determining the efficiency of a systemic AAV vector-mediated gene therapy of this disclosure, the maintaining of the infected non-human animal is done for at least 1 day. In some embodiments of the method of determining the efficiency of a systemic AAV vector-mediated gene therapy of this disclosure, the maintaining of the infected non-human animal is done for at least 1 week. In some embodiments of the method of determining the efficiency of a systemic AAV vector-mediated gene therapy of this disclosure, the maintaining of the infected non-human animal is done for at least 2 weeks. In some embodiments of the method of determining the efficiency of a systemic AAV vector-mediated gene therapy of this disclosure, the maintaining of the infected non-human animal is done for at least 4 weeks. In some embodiments of the method of determining the efficiency of a systemic AAV vector-mediated gene therapy of this disclosure, the maintaining of the infected non-human animal is done for 1 day to 5 days. In some embodiments of the method of determining the efficiency of a systemic AAV vector-mediated gene therapy of this disclosure, the maintaining of the infected non-human animal is done for 5 days to 10 days. In some embodiments of the method of determining the efficiency of a systemic AAV vector-mediated gene therapy of this disclosure, the maintaining of the infected non-human animal is done for 10 days to 15 days. In some embodiments of the method of determining the efficiency of a systemic AAV vector-mediated gene therapy of this disclosure, the maintaining of the infected non-human animal is done for 15 days to 20 days. In some embodiments of the method of determining the efficiency of a systemic AAV vector-mediated gene therapy of this disclosure, the maintaining of the infected non-human animal is done for 20 days to 25 days. In some embodiments of the method of determining the efficiency of a systemic AAV vector-mediated gene therapy of this disclosure, the maintaining of the infected non-human animal is done for 25 days to 30 days.

In some embodiments of the method of determining the efficiency of a systemic AAV vector-mediated gene therapy of this disclosure, after the first infection, the percentage of AAV vector-transduced non-human animal hepatocytes is ≤50%, ≤40%, ≤30%, ≤20%, ≤10% or ≤5%.

In some embodiments of the method of determining the efficiency of a systemic AAV vector-mediated gene therapy of this disclosure, after the first infection, the percentage of AAV vector-transduced non-human animal hepatocytes is 40% to 50%. In some embodiments of the method of determining the efficiency of a systemic AAV vector-mediated gene therapy of this disclosure, after the first infection, the percentage of AAV vector-transduced non-human animal hepatocytes is 30% to 40%. In some embodiments of the method of determining the efficiency of a systemic AAV vector-mediated gene therapy of this disclosure, after the first infection, the percentage of AAV vector-transduced non-human animal hepatocytes is 20% to 30%. In some embodiments of the method of determining the efficiency of a systemic AAV vector-mediated gene therapy of this disclosure, after the first infection, the percentage of AAV vector-transduced non-human animal hepatocytes is 10% to 20%. In some embodiments of the method of determining the efficiency of a systemic AAV vector-mediated gene therapy of this disclosure, after the first infection, the percentage of AAV vector-transduced non-human animal hepatocytes is 5% to 10%. In some embodiments of the method of determining the efficiency of a systemic AAV vector-mediated gene therapy of this disclosure, after the first infection, the percentage of AAV vector-transduced non-human animal hepatocytes is 0% to 5%.

In some embodiments of the method of determining the efficiency of a systemic AAV vector-mediated gene therapy of this disclosure, after the second infection, the percentage of AAV vector-transduced non-human animal hepatocytes is ≤50%, ≤40%, ≤30%, ≤20%, ≤10% or ≤5%.

In some embodiments of the method of determining the efficiency of a systemic AAV vector-mediated gene therapy of this disclosure, after the second infection, the percentage of AAV vector-transduced non-human animal hepatocytes is 40% to 50%. In some embodiments of the method of determining the efficiency of a systemic AAV vector-mediated gene therapy of this disclosure, after the second infection, the percentage of AAV vector-transduced non-human animal hepatocytes is 30% to 40%. In some embodiments of the method of determining the efficiency of a systemic AAV vector-mediated gene therapy of this disclosure, after the second infection, the percentage of AAV vector-transduced non-human animal hepatocytes is 20% to 30%. In some embodiments of the method of determining the efficiency of a systemic AAV vector-mediated gene therapy of this disclosure, after the second infection, the percentage of AAV vector-transduced non-human animal hepatocytes is 10% to 20%. In some embodiments of the method of determining the efficiency of a systemic AAV vector-mediated gene therapy of this disclosure, after the second infection, the percentage of AAV vector-transduced non-human animal hepatocytes is 5% to 10%. In some embodiments of the method of determining the efficiency of a systemic AAV vector-mediated gene therapy of this disclosure, after the second infection, the percentage of AAV vector-transduced non-human animal hepatocytes is 0% to 5%.

In some embodiments of the method of determining the efficiency of a systemic AAV vector-mediated gene therapy of this disclosure, after the first infection, the percentage of AAV vector-transduced human animal hepatocytes is ≥10%, ≥20%, ≥30%, ≥50%, ≥70%, ≥80% or ≥90%.

In some embodiments of the method of determining the efficiency of a systemic AAV vector-mediated gene therapy of this disclosure, after the first infection, the percentage of AAV vector-transduced human animal hepatocytes is 90% to 100%. In some embodiments of the method of determining the efficiency of a systemic AAV vector-mediated gene therapy of this disclosure, after the first infection, the percentage of AAV vector-transduced human animal hepatocytes is 80% to 90%. In some embodiments of the method of determining the efficiency of a systemic AAV vector-mediated gene therapy of this disclosure, after the first infection, the percentage of AAV vector-transduced human animal hepatocytes is 70% to 80%. In some embodiments of the method of determining the efficiency of a systemic AAV vector-mediated gene therapy of this disclosure, after the first infection, the percentage of AAV vector-transduced human animal hepatocytes is 50% to 70%. In some embodiments of the method of determining the efficiency of a systemic AAV vector-mediated gene therapy of this disclosure, after the first infection, the percentage of AAV vector-transduced human animal hepatocytes is 20% to 50%. In some embodiments of the method of determining the efficiency of a systemic AAV vector-mediated gene therapy of this disclosure, after the first infection, the percentage of AAV vector-transduced human animal hepatocytes is 10% to 20%.

In some embodiments of the method of determining the efficiency of a systemic AAV vector-mediated gene therapy of this disclosure, after the second infection, the percentage of AAV vector-transduced human hepatocytes is ≥10%, ≥20%, ≥30%, 50%, ≥70%, ≥80% or ≥90%.

In some embodiments of the method of determining the efficiency of a systemic AAV vector-mediated gene therapy of this disclosure, after the second infection, the percentage of AAV vector-transduced human animal hepatocytes is 90% to 100%. In some embodiments of the method of determining the efficiency of a systemic AAV vector-mediated gene therapy of this disclosure, after the second infection, the percentage of AAV vector-transduced human animal hepatocytes is 80% to 90%. In some embodiments of the method of determining the efficiency of a systemic AAV vector-mediated gene therapy of this disclosure, after the second infection, the percentage of AAV vector-transduced human animal hepatocytes is 70% to 80%. In some embodiments of the method of determining the efficiency of a systemic AAV vector-mediated gene therapy of this disclosure, after the second infection, the percentage of AAV vector-transduced human animal hepatocytes is 50% to 70%. In some embodiments of the method of determining the efficiency of a systemic AAV vector-mediated gene therapy of this disclosure, after the second infection, the percentage of AAV vector-transduced human animal hepatocytes is 20% to 50%. In some embodiments of the method of determining the efficiency of a systemic AAV vector-mediated gene therapy of this disclosure, after the second infection, the percentage of AAV vector-transduced human animal hepatocytes is 10% to 20%.

In some embodiments of the method of determining the efficiency of a systemic AAV vector-mediated gene therapy of this disclosure, after the first infection, the percentage of AAV vector-transduced non-human animal hepatocytes is 40% to 50%, 30% to 40%, 20% to 30%, 10% to 20%, 5% to 10% or 0% to 5%; and the percentage of AAV vector-transduced human hepatocytes is 10% to 20%, 20% to 30%, 30% to 40%, 40% to 50%, 50% to 70%, 70% to 80%, 80% to 90% or 90% to 100%.

In some embodiments of the method of determining the efficiency of a systemic AAV vector-mediated gene therapy of this disclosure, after the second infection, the percentage of AAV vector-transduced non-human animal hepatocytes is 40% to 50%, 30% to 40%, 20% to 30%, 10% to 20%, 5% to 10% or 0% to 5%; and the percentage of AAV vector-transduced human hepatocytes is 0% to 20%, 20% to 30%, 30% to 40%, 40% to 50%, 50% to 70%, 70% to 80%, 80% to 90% or 90% to 100%.

In some embodiments of the method of determining the efficiency of a systemic AAV vector-mediated gene therapy of this disclosure, the percentage of AAV vector-transduced non-human animal hepatocytes is more than the percentage of AAV vector-transduced human animal hepatocytes, after both the first and the second infections. In some embodiments of the method of determining the efficiency of a systemic AAV vector-mediated gene therapy of this disclosure, after the first infection, the percentage of AAV vector-transduced non-human animal hepatocytes is more than the percentage of AAV vector-transduced human animal hepatocytes, and after the second infection, the percentage of AAV vector-transduced non-human animal hepatocytes is same as the percentage of AAV vector-transduced human animal hepatocytes. In some embodiments of the method of determining the efficiency of a systemic AAV vector-mediated gene therapy of this disclosure, after the first infection, the percentage of AAV vector-transduced non-human animal hepatocytes is more than the percentage of AAV vector-transduced human animal hepatocytes, and after the second infection, the percentage of AAV vector-transduced non-human animal hepatocytes is less than the percentage of AAV vector-transduced human animal hepatocytes.

In some embodiments of the method of determining the efficiency of a systemic AAV-vector-mediated gene therapy of this disclosure, after the first infection, the percentage of AAV vector-transduced non-human animal hepatocytes is same as the percentage of AAV vector-transduced human animal hepatocytes, and after the second infection, the percentage of AAV vector-transduced non-human animal hepatocytes is less than the percentage of AAV vector-transduced human animal hepatocytes. In some embodiments of the method of determining the efficiency of a systemic AAV vector-mediated gene therapy of this disclosure, after the first infection, the percentage of AAV vector-transduced non-human animal hepatocytes is same as the percentage of AAV vector-transduced human animal hepatocytes, and after the second infection, the percentage of AAV vector-transduced non-human animal hepatocytes is more than the percentage of AAV vector-transduced human animal hepatocytes.

In some embodiments of the method of determining the efficiency of a systemic AAV vector-mediated gene therapy of this disclosure, after the first infection, the percentage of AAV vector-transduced non-human animal hepatocytes is less than the percentage of AAV vector-transduced human animal hepatocytes, and after the second infection, the percentage of AAV vector-transduced non-human animal hepatocytes is the same as the percentage of AAV vector-transduced human animal hepatocytes. In some embodiments of the method of determining the efficiency of a systemic AAV vector-mediated gene therapy of this disclosure, after the first infection, the percentage of AAV vector-transduced non-human animal hepatocytes is less than the percentage of AAV vector-transduced human animal hepatocytes, and after the second infection, the percentage of AAV vector-transduced non-human animal hepatocytes is more than the percentage of AAV vector-transduced human animal hepatocytes.

In some embodiments, of the method of determining the efficiency of a systemic AAV vector-mediated gene therapy of this disclosure, the percentage of AAV vector-transduced human animal hepatocytes after the second infection is higher than after the first infection. In some embodiments, of the method of determining the efficiency of a systemic AAV vector-mediated gene therapy of this disclosure, the percentage of AAV vector-transduced non-human animal hepatocytes after the second infection is higher than after the first infection.

The present disclosure also provides a method of determining Adeno-associated virus receptor (AAVR)-dependent or AAVR-independent transduction efficiency of an AAV vector in human hepatocytes, wherein the method comprises: (a) providing two or more chimeric non-human animals prepared by any one of the methods of the disclosure, divided into two groups, Group A and Group B, each group comprising one or more non-human animals, wherein the Group A receives wild type human hepatocytes and Group B receives human hepatocytes comprising a deletion or mutation of AAVR resulting in a non-functional human AAVR (Hu AAVR KO human hepatocytes); (b) infecting the one or more non-human animal of each group of (a) with an AAV vector; (c) maintaining the one or more non-human animal of each group of (b) for an amount of time; and (d) determining the level of transduction of the AAV vector into the human hepatocytes of the one or more non-human animals of Group A and Group B, wherein the level of transduction in Group A is indicative of AAVR-dependent transduction efficiency and the level of transduction in Group B is indicative of AAVR-independent transduction efficiency, of the AAV vector.

In some embodiments of the method of determining AAVR-dependent or AAVR-independent transduction efficiency of an AAV vector in human hepatocytes of this disclosure, step (a) further comprises applying selection pressure. In some embodiments of the methods of this disclosure, applying a selection pressure comprises not providing nitisinone (NTBC) to the non-human animal of step (a). In some embodiments of the method of determining AAVR-dependent or AAVR-independent transduction efficiency of an AAV vector in human hepatocytes of this disclosure, the method further comprises removing the selection pressure following step (b). In some embodiments, the removal of the selection pressure can comprise providing NTBC to the chimeric non-human animal following step (b) of the methods disclosed herein.

In some embodiments of the method of determining AAVR-dependent or AAVR-independent transduction efficiency of an AAV vector in human hepatocytes of this disclosure, the level of transduction of the AAV vector into the human hepatocytes of non-human animal in Group A and Group B, is determined by the percentage of AAV vector-transduced human animal hepatocytes in Group A and Group B, respectively. In some embodiments of the method of determining AAVR-dependent or AAVR-independent transduction efficiency of an AAV vector in human hepatocytes of this disclosure, the percentage of AAV vector-transduced human hepatocytes in Group A is ≥0%, ≥5%, ≥10%, ≥20%, ≥30%, ≥50%, ≥70%, ≥80% or ≥90%. In some embodiments of the method of determining AAVR-dependent or AAVR-independent transduction efficiency of an AAV vector in human hepatocytes of this disclosure, the percentage of AAV vector-transduced human hepatocytes in Group A is 10% to 20%, 20% to 30%, 30% to 40%, 40% to 50%, 50% to 70%, 70% to 80%, 80% to 90% or 90% to 100%.

In some embodiments of the method of determining AAVR-dependent or AAVR-independent transduction efficiency of an AAV vector in human hepatocytes of this disclosure, the percentage of AAV vector-transduced human hepatocytes in Group B is ≥0%, ≥5%, ≥10%, ≥20%, ≥30%, ≥50%, ≥70%, ≥80% or ≥90%. In some embodiments of the method of determining AAVR-dependent or AAVR-independent transduction efficiency of an AAV vector in human hepatocytes of this disclosure, the percentage of AAV vector-transduced human hepatocytes in Group B is 0% to 5%, 5% to 10%, 10% to 20%, 20% to 30%, 30% to 40%, 40% to 50%, 50% to 70%, 70% to 80%, 80% to 90% or 90% to 100%.

In some embodiments of the method of determining AAVR-dependent or AAVR-independent transduction efficiency of an AAV vector in human hepatocytes of this disclosure, the percentage of AAV vector-transduced human animal hepatocytes in Group A is higher than the percentage of AAV vector-transduced human animal hepatocytes in Group B, indicating that the AAV transduction is more AAVR-dependent than AAVR-independent. In some embodiments of the method of determining AAVR-dependent or AAVR-independent transduction efficiency of an AAV vector in human hepatocytes of this disclosure, the percentage of AAV vector-transduced human animal hepatocytes in Group A is lower than the percentage of AAV vector-transduced human animal hepatocytes in Group B, indicating that the AAV transduction is more AAVR-independent than AAVR-dependent. In some embodiments of the method of determining AAVR-dependent or AAVR-independent transduction efficiency of an AAV vector in human hepatocytes of this disclosure, the percentage of AAV vector-transduced human animal hepatocytes in Group A is same as the percentage of AAV vector-transduced human animal hepatocytes in Group B, indicating that the AAV transduction is equally AAVR-dependent than AAVR-independent.

In some embodiments of the method of determining AAVR-dependent or AAVR-independent transduction efficiency of an AAV vector in human hepatocytes of this disclosure, the percentage of AAV vector-transduced human hepatocytes in Group A is ≥5%, ≥10%, ≥20%, ≥30%, ≥50%, ≥70%, ≥80% or ≥90% and the percentage of AAV vector-transduced human hepatocytes in Group B is 0% to 5%, indicating that the AAV transduction is AAVR-dependent. In some embodiments of the method of determining AAVR-dependent or AAVR-independent transduction efficiency of an AAV vector in human hepatocytes of this disclosure, the percentage of AAV vector-transduced human hepatocytes in Group A is 0% to 5% and the percentage of AAV vector-transduced human hepatocytes in Group B is 5%, ≥10%, ≥20%, ≥30%, ≥50%, ≥70%, ≥80% or ≥90%, indicating that the AAV transduction is AAVR-independent.

The AAVR-dependent and AAVR-independent transduction of an AAV vector into human hepatocytes of Group A and Group B, respectively, of the present disclosure depend on the serotype or variant of the AAV. The AAVR-dependent and AAVR-independent transduction of an AAV vector into human hepatocytes of Group A and Group B, respectively, of the present disclosure depend on the specific capsid proteins of the AAV. In some embodiments, the transduction of the AAV vector into a human hepatocyte can be mediated only by AAVR. In some embodiments, the transduction of the AAV vector into a human hepatocyte can be mediated only by one or more receptors other than AAVR (non-AAVR receptors). In some embodiments, the transduction of the AAV vector into a human hepatocyte can be mediated by both AAVR and one or more receptors other than AAVR (non-AAVR receptors).

In some embodiments of the method of determining AAVR-dependent or AAVR-independent transduction efficiency of an AAV vector in human hepatocytes of this disclosure, the number of non-human animals of Group A and Group B are equal. In some embodiments of the method of determining AAVR-dependent or AAVR-independent transduction efficiency of an AAV vector in human hepatocytes of this disclosure, the number of non-human animals of Group A and Group B are different.

In some embodiments of the method of determining AAVR-dependent or AAVR-independent transduction efficiency of an AAV vector in human hepatocytes of this disclosure, the amount of AAV vector in the infection is 1×10¹ to 1×10⁵ viral genomes per non-human animal. In some embodiments of the method of determining AAVR-dependent or AAVR-independent transduction efficiency of an AAV vector in human hepatocytes of this disclosure, the amount of AAV vector in the infection is 1×10² to 1×10¹² viral genomes per non-human animal. In some embodiments of the method of determining AAVR-dependent or AAVR-independent transduction efficiency of an AAV vector in human hepatocytes of this disclosure, the amount of AAV vector in the infection is 1×10⁴ to 1×10¹⁰ viral genomes per non-human animal. In some embodiments of the method of determining AAVR-dependent or AAVR-independent transduction efficiency of an AAV vector in human hepatocytes of this disclosure, the amount of AAV vector in the infection is 1×10⁶ to 1×10⁸ viral genomes per non-human animal. In some embodiments of the method of determining AAVR-dependent or AAVR-independent transduction efficiency of an AAV vector in human hepatocytes of this disclosure, the amount of AAV vector in the infection is 1×10¹ to 1×10³ viral genomes per non-human animal. In some embodiments of the method of determining AAVR-dependent or AAVR-independent transduction efficiency of an AAV vector in human hepatocytes of this disclosure, the amount of AAV vector in the infection is 1×10² to 1×10⁵ viral genomes per non-human animal. In some embodiments of the method of determining AAVR-dependent or AAVR-independent transduction efficiency of an AAV vector in human hepatocytes of this disclosure, the amount of AAV vector in the infection is 1×10⁵ to 1×10⁸ viral genomes per non-human animal. In some embodiments of the method of determining AAVR-dependent or AAVR-independent transduction efficiency of an AAV vector in human hepatocytes of this disclosure, the amount of AAV vector in the infection is 1×10⁸ to 1×10^{1V} viral genomes per non-human animal. In some embodiments of the method of determining AAVR-dependent or AAVR-independent transduction efficiency of an AAV vector in human hepatocytes of this disclosure, the amount of AAV vector in the infection is 1×10¹² to 1×10¹⁵ viral genomes per non-human animal.

In some embodiments of the method of determining AAVR-dependent or AAVR-independent transduction efficiency of an AAV vector in human hepatocytes of this disclosure, the maintaining of the infected non-human animal is done for at least 1 day. In some embodiments of the method of determining AAVR-dependent or AAVR-independent transduction efficiency of an AAV vector in human hepatocytes of this disclosure, the maintaining of the infected non-human animal is done for at least 1 week. In some embodiments of the method of determining AAVR-dependent or AAVR-independent transduction efficiency of an AAV vector in human hepatocytes of this disclosure, the maintaining of the infected non-human animal is done for at least 2 weeks. In some embodiments of the method of determining AAVR-dependent or AAVR-independent transduction efficiency of an AAV vector in human hepatocytes of this disclosure, the maintaining of the infected non-human animal is done for at least 4 weeks. In some embodiments of the method of determining AAVR-dependent or AAVR-independent transduction efficiency of an AAV vector in human hepatocytes of this disclosure, the maintaining of the infected non-human animal is done for 1 day to 5 days. In some embodiments of the method of determining AAVR-dependent or AAVR-independent transduction efficiency of an AAV vector in human hepatocytes of this disclosure, the maintaining of the infected non-human animal is done for 5 days to 10 days. In some embodiments of the method of determining AAVR-dependent or AAVR-independent transduction efficiency of an AAV vector in human hepatocytes of this disclosure, the maintaining of the infected non-human animal is done for 10 days to 15 days. In some embodiments of the method of determining AAVR-dependent or AAVR-independent transduction efficiency of an AAV vector in human hepatocytes of this disclosure, the maintaining of the infected non-human animal is done for 15 days to 20 days. In some embodiments of the method of determining AAVR-dependent or AAVR-independent transduction efficiency of an AAV vector in human hepatocytes of this disclosure, the maintaining of the infected non-human animal is done for 20 days to 25 days. In some embodiments of the method of determining AAVR-dependent or AAVR-independent transduction efficiency of an AAV vector in human hepatocytes of this disclosure, the maintaining of the infected non-human animal is done for 25 days to 30 days.

In some embodiments of the method of determining AAVR-dependent or AAVR-independent transduction efficiency of an AAV vector in human hepatocytes of this disclosure, the AAV is any one of the AAV serotypes 1, 2, 3, 4, 5, 6, 7, 8, 9, 10 and 11. In some embodiments of the method of determining AAVR-dependent or AAVR-independent transduction efficiency of an AAV vector in human hepatocytes of this disclosure, the AAV is AAV serotype 8. In some embodiments of the method of determining AAVR-dependent or AAVR-independent transduction efficiency of an AAV vector in human hepatocytes of this disclosure, the AAV is AAV serotype 9. In some embodiments of the method of determining AAVR-dependent or AAVR-independent transduction efficiency of an AAV vector in human hepatocytes of this disclosure, the AAV is a AAV serotype that specifically infects or transduces the liver of a subject. In some embodiments of the method of determining AAVR-dependent or AAVR-independent transduction efficiency of an AAV vector in human hepatocytes of this disclosure, the AAV is a AAV serotype that specifically infects or transduces hepatocytes. In some embodiments of the method of determining AAVR-dependent or AAVR-independent transduction efficiency of an AAV vector in human hepatocytes of this disclosure, the AAV is a hybrid of two or more AAV serotypes. In some embodiments of the method of determining AAVR-dependent or AAVR-independent transduction efficiency of an AAV vector in human hepatocytes of this disclosure, the AAV is a variant selected from 6.2, 2, rh64R1, rh10, 8, 9 and AAV9-PHP.B. In some embodiments of the method of determining AAVR-dependent or AAVR-independent transduction efficiency of an AAV vector in human hepatocytes of this disclosure, the AAV vector encodes a detectable marker. In some embodiments of the method of determining AAVR-dependent or AAVR-independent transduction efficiency of an AAV vector in human hepatocytes of this disclosure, the detectable marker is a fluorescent protein, an enzyme, or a peptide. In some embodiments of the method of determining AAVR-dependent or AAVR-independent transduction efficiency of an AAV vector in human hepatocytes of this disclosure, the detectable marker is GFP, RFP, YFP, CFP, dTomato, mCherry or LacZ (3-galactosidase). In some embodiments of the method of determining AAVR-dependent or AAVR-independent transduction efficiency of an AAV vector in human hepatocytes of this disclosure, the AAV vector comprises an inducible promoter or a constitutive promoter. In some embodiments of the method of determining AAVR-dependent or AAVR-independent transduction efficiency of an AAV vector in human hepatocytes of this disclosure, the AAV vector comprises a tissue specific promoter. In some embodiments of the method of determining AAVR-dependent or AAVR-independent transduction efficiency of an AAV vector in human hepatocytes of this disclosure, the inducible promoter is a tetracycline-inducible promoter or a CMV promoter. In some embodiments of the method of determining AAVR-dependent or AAVR-independent transduction efficiency of an AAV vector in human hepatocytes of this disclosure, the AAV vector encodes one or more heterologous proteins. In some embodiments of the method of determining AAVR-dependent or AAVR-independent transduction efficiency of an AAV vector in human hepatocytes of this disclosure, the one or more heterologous proteins can be selected from an immunogenic protein or peptide, a therapeutic protein, a regulatory protein or a marker/detectable protein.

The present disclosure also provides a method of determining AAVR-dependent or AAVR-independent modification and/or inhibition of an AAV vector transduction into human hepatocytes, wherein the method comprises: (a) providing two groups of non-human animals, Group A and Group B, wherein Group A comprises one or more a T-, B- and NK cell deficient or impaired in function non-human animals, and Group B comprises one or a T, B- and NK cell deficient or impaired in function non-human animals, further comprising a deletion or mutation of AAVR resulting in a non-functional non-human animal AAVR; (b) transplanting human hepatocytes into the one or more non-human animals of both groups of (a); (c) infecting the non-human animals of both groups of (b) with an AAV vector; (d) maintaining the one or more infected non-human animal of each group of (c) for an amount of time; and (e) determining the level of transduction of the AAV vector into the human hepatocytes and non-human hepatocytes of the non-human animals of Group A and Group B.

The “modification” of an AAV vector transaction comprises, for example, a change in the timing or efficacy or transduction, and/or a change in the type of cell population transfected with the AAV vector.

The present disclosure also provides a method of determining AAVR-dependent or AAVR-independent modification and/or inhibition of an AAV vector transduction into human hepatocytes, wherein the method comprises: (a) providing two groups of non-human animals, Group A and Group B, wherein Group A comprises one or more IL-2Rg−/−/Rag 2−/− non-human animals, and Group B comprises one or more IL-2Rg−/−/Rag 2−/− non-human animals, further comprising a deletion or mutation of AAVR resulting in a non-functional non-human animal AAVR; (b) transplanting human hepatocytes into the one or more non-human animals of both groups of (a) and applying selection pressure; (c) infecting the non-human animals of both groups of (b) with an AAV vector; (d) maintaining the one or more infected non-human animal of each group of (c) for an amount of time; and (e) determining the level of transduction of the AAV vector into the human hepatocytes and non-human hepatocytes of the non-human animals of Group A and Group B. In some embodiments of the methods of this disclosure, the non-human animal further comprises a Fah−/−. In some embodiments of the methods of determining AAVR-dependent or AAVR-independent modification and/or inhibition of an AAV vector transduction into human hepatocytes of the present disclosure, step (b) further comprises applying a selection pressure. In some embodiments of the methods of the disclosure, applying a selection pressure comprises not providing nitisinone (NTBC) to the non-human animal of step (b). In some embodiments of the methods of the disclosure, the method further comprises removing the selection pressure following step (b). In some embodiments, the removal of the selection pressure can comprise providing NTBC to the chimeric non-human animal following step (b) of the methods disclosed herein.

In some embodiments of the method of determining AAVR-dependent or AAVR-independent modification and/or inhibition of an AAV vector transduction into human hepatocytes of this disclosure, the method further comprises: (f) comparing the level of transduction of the AAV vector into the human hepatocytes and non-human hepatocytes of the non-human animals between Group A and Group B, (i) wherein the difference between the levels of transduction of AAV into the non-human hepatocytes and the human hepatocytes of Group A is indicative of both AAVR-dependent and AAVR-independent modification and/or inhibition of uptake of the AAV vector into human hepatocytes; (ii) wherein the difference between the levels of transduction of AAV into the non-human hepatocytes and the human hepatocytes of Group B is indicative of AAVR-independent modification and/or inhibition of uptake of the AAV vector into human hepatocytes; and (iii) wherein the difference between the levels of transduction of AAV into human hepatocytes in Group B and Group A is indicative of AAVR-dependent modification and/or inhibition of uptake of the AAV vector into human hepatocytes.

In some embodiments of the method of determining AAVR-dependent or AAVR-independent modification and/or inhibition of an AAV vector transduction into human hepatocytes of this disclosure, the level of transduction of the AAV vector into the human hepatocytes of non-human animal in Group A and Group B, is determined by the percentage of AAV vector-transduced human hepatocytes in Group A and Group B, respectively.

In some embodiments of the method of determining AAVR-dependent or AAVR-independent modification and/or inhibition of an AAV vector transduction into human hepatocytes of this disclosure, the percentage of AAV vector-transduced non-human animal hepatocytes in Group A is ≥0%, ≥5%, ≥10%, ≥20%, ≥30%, ≥50%, ≥70%, ≥80% or ≥90%. In some embodiments of the method of determining AAVR-dependent or AAVR-independent modification and/or inhibition of an AAV vector transduction into human hepatocytes of this disclosure, the percentage of AAV vector-transduced non-human animal hepatocytes in Group A is 0% to 5%, 5% to 10%, 10% to 20%, 20% to 30%, 30% to 40%, 40% to 50%, 50% to 70%, 70% to 80%, 80% to 90% or 90% to 100%.

In some embodiments of the method of determining AAVR-dependent or AAVR-independent modification and/or inhibition of an AAV vector transduction into human hepatocytes of this disclosure, the percentage of AAV vector-transduced human hepatocytes in Group A is ≥0%, ≥5%, ≥10%, ≥20%, ≥30%, ≥50%, ≥70%, ≥80% or ≥90%. In some embodiments of the method of determining AAVR-dependent or AAVR-independent modification and/or inhibition of an AAV vector transduction into human hepatocytes of this disclosure, the percentage of AAV vector-transduced human hepatocytes in Group A is 0% to 5%, 5% to 10%, 10% to 20%, 20% to 30%, 30% to 40%, 40% to 50%, 50% to 70%, 70% to 80%, 80% to 90% or 90% to 100%.

In some embodiments of the method of determining AAVR-dependent or AAVR-independent modification and/or inhibition of an AAV vector transduction into non-human hepatocytes of this disclosure, the percentage of AAV vector-transduced non-human animal hepatocytes in Group B is ≥0%, ≥5%, ≥10%, ≥20%, ≥30%, ≥50%, ≥70%, ≥80% or ≥90%. In some embodiments of the method of determining AAVR-dependent or AAVR-independent modification and/or inhibition of an AAV vector transduction into non-human animal hepatocytes of this disclosure, the percentage of AAV vector-transduced non-human animal hepatocytes in Group B is 0% to 5%, 5% to 10%, 10% to 20%, 20% to 30%, 30% to 40%, 40% to 50%, 50% to 70%, 70% to 80%, 80% to 90% or 90% to 100%.

In some embodiments of the method of determining AAVR-dependent or AAVR-independent modification and/or inhibition of an AAV vector transduction into human hepatocytes of this disclosure, the percentage of AAV vector-transduced human hepatocytes in Group B is ≥0%, ≥5%, ≥10%, ≥20%, ≥30%, ≥50%, ≥70%, ≥80% or ≥90%. In some embodiments of the method of determining AAVR-dependent or AAVR-independent modification and/or inhibition of an AAV vector transduction into human hepatocytes of this disclosure, the percentage of AAV vector-transduced human hepatocytes in Group B is 0% to 5%, 5% to 10%, 10% to 20%, 20% to 30%, 30% to 40%, 40% to 50%, 50% to 70%, 70% to 80%, 80% to 90% or 90% to 100%.

In some embodiments of the method of determining AAVR-dependent or AAVR-independent modification and/or inhibition of an AAV vector transduction into human hepatocytes of this disclosure, the difference between the levels of transduction of AAV into the non-human hepatocytes and the human hepatocytes of Group A in (i), the difference between the levels of transduction of AAV into the non-human hepatocytes and the human hepatocytes of Group B in (ii) and the difference between the levels of transduction of AAV into human hepatocytes in Group B and Group A, can be expressed or indicated as percentage difference or fold change.

The AAVR-dependent or AAVR-independent modification and/or inhibition of an AAV vector transduction into human hepatocytes depends on the AAV serotype or variant. The AAVR-dependent or AAVR-independent modification and/lor inhibition of an AAV vector transduction into human hepatocytes depends on the specific capsid proteins of the AAV vector.

In some embodiments of the method of determining AAVR-dependent or AAVR-independent modification and/or inhibition of an AAV vector transduction into human hepatocytes of this disclosure, the number of non-human animals in Group A and Group B is same. In some embodiments of the method of determining AAVR-dependent or AAVR-independent modification and/or inhibition of an AAV vector transduction into human hepatocytes of this disclosure, the number of non-human animals in Group A and Group B are different.

In some embodiments of the method of determining AAVR-dependent or AAVR-independent modification and/or inhibition of an AAV vector transduction into human hepatocytes of this disclosure, the amount of AAV vector in the infection is 1×10¹ to 1×10¹⁵ viral genomes per non-human animal. In some embodiments of the method of determining AAVR-dependent or AAVR-independent modification and/or inhibition of an AAV vector transduction into human hepatocytes of this disclosure, the amount of AAV vector in the infection is 1×10² to 1×10¹² viral genomes per non-human animal. In some embodiments of the method of determining AAVR-dependent or AAVR-independent modification and/or inhibition of an AAV vector transduction into human hepatocytes of this disclosure, the amount of AAV vector in the infection is 1×10⁴ to 1×10¹⁰ viral genomes per non-human animal. In some embodiments of the method of determining AAVR-dependent or AAVR-independent modification and/or inhibition of an AAV vector transduction into human hepatocytes of this disclosure, the amount of AAV vector in the infection is 1×10⁶ to 1×10⁸ viral genomes per non-human animal. In some embodiments of the method of determining AAVR-dependent or AAVR-independent modification and/or inhibition of an AAV vector transduction into human hepatocytes of this disclosure, the amount of AAV vector in the infection is 1×10¹ to 1×10³ viral genomes per non-human animal. In some embodiments of the method of determining AAVR-dependent or AAVR-independent modification and/or inhibition of an AAV vector transduction into human hepatocytes of this disclosure, the amount of AAV vector in the infection is 1×10² to 1×10⁵ viral genomes per non-human animal. In some embodiments of the method of determining AAVR-dependent or AAVR-independent modification and/or inhibition of an AAV vector transduction into human hepatocytes of this disclosure, the amount of AAV vector in the infection is 1×10⁵ to 1×10⁸ viral genomes per non-human animal. In some embodiments of the method of determining AAVR-dependent or AAVR-independent modification and/or inhibition of an AAV vector transduction into human hepatocytes of this disclosure, the amount of AAV vector in the infection is 1×10⁸ to 1×10¹² viral genomes per non-human animal. In some embodiments of the method of determining AAVR-dependent or AAVR-independent modification and/or inhibition of an AAV vector transduction into human hepatocytes of this disclosure, the amount of AAV vector in the infection is 1×10¹² to 1×10¹⁵ viral genomes per non-human animal.

In some embodiments of the method of determining AAVR-dependent or AAVR-independent modification and/or inhibition of an AAV vector transduction into human hepatocytes of this disclosure, the maintaining of the infected non-human animal is done for at least 1 day. In some embodiments of the method of determining AAVR-dependent or AAVR-independent modification and/or inhibition of an AAV vector transduction into human hepatocytes of this disclosure, the maintaining of the infected non-human animal is done for at least 1 week. In some embodiments of the method of determining AAVR-dependent or AAVR-independent modification and/or inhibition of an AAV vector transduction into human hepatocytes of this disclosure, the maintaining of the infected non-human animal is done for at least 2 weeks. In some embodiments of the method of determining AAVR-dependent or AAVR-independent modification and/or inhibition of an AAV vector transduction into human hepatocytes of this disclosure, the maintaining of the infected non-human animal is done for at least 4 weeks. In some embodiments of the method of determining AAVR-dependent or AAVR-independent modification and/or inhibition of an AAV vector transduction into human hepatocytes of this disclosure, the maintaining of the infected non-human animal is done for 1 day to 5 days. In some embodiments of the method of determining AAVR-dependent or AAVR-independent modification and/or inhibition of an AAV vector transduction into human hepatocytes of this disclosure, the maintaining of the infected non-human animal is done for 5 days to 10 days. In some embodiments of the method of determining AAVR-dependent or AAVR-independent modification and/or inhibition of an AAV vector transduction into human hepatocytes of this disclosure, the maintaining of the infected non-human animal is done for 10 days to 15 days. In some embodiments of the method of determining AAVR-dependent or AAVR-independent modification and/or inhibition of an AAV vector transduction into human hepatocytes of this disclosure, the maintaining of the infected non-human animal is done for 15 days to 20 days. In some embodiments of the method of determining AAVR-dependent or AAVR-independent modification and/or inhibition of an AAV vector transduction into human hepatocytes of this disclosure, the maintaining of the infected non-human animal is done for 20 days to 25 days. In some embodiments of the method of determining AAVR-dependent or AAVR-independent modification and/or inhibition of an AAV vector transduction into human hepatocytes of this disclosure, the maintaining of the infected non-human animal is done for 25 days to 30 days.

In some embodiments of the method of determining AAVR-dependent or AAVR-independent modification and/or inhibition of an AAV vector transduction into human hepatocytes of this disclosure, the AAV is any one of the AAV serotypes 1, 2, 3, 4, 5, 6, 7, 8, 9, 10 and 11. In some embodiments of the method of determining AAVR-dependent or AAVR-independent modification and/or inhibition of an AAV vector transduction into human hepatocytes of this disclosure, the AAV is AAV serotype 8. In some embodiments of the method of determining AAVR-dependent or AAVR-independent modification and/or inhibition of an AAV vector transduction into human hepatocytes of this disclosure, the AAV is AAV serotype 9. In some embodiments of the method of determining AAVR-dependent or AAVR-independent modification and/lor inhibition of an AAV vector transduction into human hepatocytes of this disclosure, the AAV is a AAV serotype that specifically infects or transduces the liver of a subject. In some embodiments of the method of determining AAVR-dependent or AAVR-independent modification and/or inhibition of an AAV vector transduction into human hepatocytes of this disclosure, the AAV is a AAV serotype that specifically infects or transduces hepatocytes. In some embodiments of the method of determining AAVR) dependent or AAVR-independent modification and/or inhibition of an AAV vector transduction into human hepatocytes of this disclosure, the AAV is a hybrid of two or more AAV serotypes. In some embodiments of the method of determining Adeno-associated virus receptor (AAVR-dependent or AAVR-independent modification and/or inhibition of an AAV vector transduction into human hepatocytes of this disclosure, the AAV is a variant selected from any one of 6.2, 2, rh64R1, rh10, 8, 9 and AAV9-PHP.B. In some embodiments of the method of determining AAVR-dependent or AAVR-independent modification and/or inhibition of an AAV vector transduction into human hepatocytes of this disclosure, the AAV vector encodes a detectable marker. In some embodiments of the method of determining AAVR-dependent or AAVR-independent modification and/or inhibition of an AAV vector transduction into human hepatocytes of this disclosure, the detectable marker is a fluorescent protein, an enzyme, or a peptide. In some embodiments of the m method of determining AAVR-dependent or AAVR-independent modification and/or inhibition of an AAV vector transduction into human hepatocytes of this disclosure, the detectable marker is GFP, RFP, YFP, CFP, dTomato, mCherry or LacZ (β-galactosidase). In some embodiments of the method of determining AAVR-dependent or AAVR-independent modification and/or inhibition of an AAV vector transduction into human hepatocytes of this disclosure, the AAV vector comprises an inducible promoter or a constitutive promoter. In some embodiments of the method of determining AAVR-dependent or AAVR-independent modification and/or inhibition of an AAV vector transduction into human hepatocytes of this disclosure, the AAV vector comprises a tissue specific promoter. In some embodiments of the method of determining AAVR-dependent or AAVR-independent modification and/or inhibition of an AAV vector transduction into human hepatocytes of this disclosure, the inducible promoter is a tetracycline-inducible promoter or a CMV promoter. In some embodiments of the method of determining AAVR-dependent or AAVR-independent modification and/or inhibition of an AAV vector transduction into human hepatocytes of this disclosure, the AAV vector encodes one or more heterologous proteins. In some embodiments of the method of determining AAVR-dependent or AAVR-independent modification and/or inhibition of an AAV vector transduction into human hepatocytes of this disclosure, the one or more heterologous proteins can be an immunogenic protein or peptide, a therapeutic protein, a regulatory protein or a marker/detectable protein.

Without wishing to be bound by theory, the disclosed chimeric non-human or humanized animal (e.g., mice) unite the experimental tractability of the non-human animal with the physiological accuracy of human hepatocytes and thus allow for validation of results from a range of studies with disparate methodologies to determine the transduction efficacy of AAV vectors into human hepatocytes. It is believed that these mice are useful for mechanistic studies on the molecular basis of the disease and for pre-clinical testing of experimental therapies. In some embodiments, the chimeric non-human animal of the present disclosure can be any one of a primate, bird, mouse, rat, fowl, dog, cat, cow, horse, goat, camel, sheep and pig.

In another aspect, provided herein is a chimeric non-human animal comprising one or more human tissues, wherein the chimeric non-human animal further comprises: (a) a T-, B- and/or NK cell deficiency or functional impairment that allows re-populating with one or more human tissues to establish human chimerism in the non-human animal; and (b) a deletion or mutation of Adeno-associated virus receptor (AAVR) resulting in deficiency or functional impairment of the non-human animal AAVR. In some embodiments, the chimeric non-human animal further comprises a Fah−/−. In some embodiments, the chimeric non-human animal is a IL-2Rg^{-/-}/Rag 2^{-/-}/Fah^{-/-} non-human animal. Also provided herein is a chimeric non-human animal comprising one or more human tissues, wherein the chimeric non-human animal is a IL-2Rg^{-/-}/Rag 2^{-/-} chimeric non-human animal, and wherein the chimeric non-human animal further comprises a deletion or mutation of AAVR resulting in deficiency or functional impairment of the non-human animal AAVR.

The one or more human tissue in any of the non-human animals provided herein may be any tissue that is suitable for transplantation into a non-human animal. In some embodiments, the one or more human tissue is connective tissue, epithelial tissue, muscle tissue (e.g., smooth muscle tissue, skeletal muscle tissue, or cardiac muscle tissue), or nervous tissue. In some embodiments, the tissue is early neural, mesodermal, endodermal, or ectodermal tissue. In some embodiments, the human tissue is liver tissue, kidney tissue, pancreatic tissue, intestinal tissue, spleen tissue, cardiac tissue, lung tissue, skin tissue, bone tissue, eye tissue, hematopoietic tissue, lymph tissue, reproductive tissue, mucosal tissue, or gastric tissue.

The one or more human tissue may be introduced into the non-human animal by any suitable means. For example, the non-human animal may be transplanted with induced pluripotent stem cells which then differentiate into the one or more human tissue. In some embodiments, the one or more human tissue are present in a teratoma.

In another aspect, provided herein is a method for preparing a chimeric non-human animal comprising one or more human tissues, the method comprising: (a) providing a T-, B- and/or NK cell deficient or impaired in function non-human animal, that allows re-populating with human tissue to establish human chimerism in the non-human animal, wherein the non-human animal comprises a deletion or mutation of AAVR resulting in a non-functional non-human animal AAVR; and (b) transplanting one or more human tissues into the non-human animal. In some embodiments, the non-human animal further comprises a Fah−/−. In some embodiments, the non-human animal is a IL-2Rg^{-/-}/Rag 2^{-/-}/Fah^{-/-} non-human animal.

Also provided herein is a method for preparing a chimeric non-human animal comprising one or more human tissues, the method comprising: (a) providing a IL-2Rg^{-/-}/Rag 2^{-/-} non-human animal, wherein the non-human animal comprises a deletion or mutation of Adeno-associated virus receptor (AAVR) resulting in a non-functional non-human animal AAVR; and (b) transplanting human tissue into the non-human animal. In some embodiments, the non-human animal further comprises a Fah^{-/-}.

In another aspect, provided herein is a method of determining transduction efficiency of an AAV vector in one or more human tissues, wherein the method comprises: (a) providing a non-human animal described herein; (b) infecting the non-human animal of (a) with an amount of the AAV vector; and (c) determining the level of transduction of the AAV vector into the human tissue of the AAVR KO non-human animal.

Also provided herein is a method of determining transduction efficiency of two or more non-identical AAV vector(s) in at least two one or more human tissues, wherein the method comprises: (a) providing two or more AAVR KO non-human animal described herein, (b) infecting each of the non-human animals of (a) with an amount of the two or more non-identical AAV vectors, wherein each non-human animal is infected with one AAV vector; and (c) determining the level of transduction of the AAV vector into at least one human tissue in each of the two or more non-human animals of (b). In some embodiments, the method further comprises (d) comparing the level of transduction of each of the two or more AAV vectors into the one or more human tissue. In some embodiments, the method further comprises (f) selecting one or more AAV vector(s) as efficient in transducing human hepatocytes if: (i) less than a pre-determined percentage of the total amount of the AAV vector is transduced into a non-human tissues of the non-human animal; (ii) at least a pre-determined percentage of the total amount of AAV vectors is transduced into the one or more human tissue; (iii) percentage of AAV vector-transduced-non-human tissue is less than a pre-determined value; and/or (iv) percentage of AAV vector-transduced human tissue is more than a pre-determined value.

In some embodiments of the methods disclosed herein, the AAV vector expresses a detectable marker, e.g., a fluorescent protein, an enzyme, or a peptide. In some embodiments, the AAV vector comprises a unique nucleic acid sequence in the genome of the vector, wherein the unique nucleic acid sequence can transcribe into a detectable or a barcode RNA sequence.

Also provided herein is a method of determining the efficiency of a systemic AAV vector-mediated gene therapy, wherein the method comprises: (a) providing a non-human animal described herein; (b) infecting the non-human animal of (a) with an amount of an AAV vector, at least a first time; and (c) determining the level of transduction of the AAV vector into the one or more human tissues of the AAVR KO non-human animal. In some embodiments, the method further comprises the method comprises: (d) infecting the non-human animal with an amount of an AAV vector, a second time; and (e) determining the level of transduction of the AAV vector into the one or more human tissue of the AAVR KO non-human animal. In some embodiments, the method further comprises (f) comparing the level of transduction of the AAV vector into the one or more human tissue between the first and the second infection. In some embodiments, the amount of AAV vector in the first and/or second infection is 1×10¹ to 1×10¹⁵ viral genomes per non-human animal. In some embodiments, the amounts of AAV vector in the first and the second infection are same. In some embodiments, amounts of AAV vector in the first and the second infection are different.

In another aspect, provided herein is a method of determining AAVR-dependent or AAVR-independent transduction efficiency of an AAV vector in one or more human tissues, wherein the method comprises. (a) providing non-human animals divided into two groups, Group A and Group B, each group comprising one or more non-human animals, wherein the Group A is transplanted with wild type human tissue and Group B is transplanted with human tissue comprising a deletion or mutation of Adeno-associated virus receptor (AAVR) resulting in a non-functional human AAVR, wherein the two or more non-human animals further comprise a T-, B- and/or NK cell deficiency or impairment in function, IL-2Rg−/−/Rag 2−/−/, and/or Fah−/−. (b) infecting the one or more non-human animal of each group of (a) with an AAV vector; (c) determining the level of transduction of the AAV vector into one or more human tissue of non-human animal of Group A and Group B, wherein the level of transduction in Group A is indicative of AAVR-dependent transduction efficiency and the level of transduction in Group B is indicative of AAVR-independent transduction efficiency, of the AAV vector.

In another aspect, provided herein is a method of determining Adeno-associated virus receptor (AAVR) dependent or AAVR-independent modification and/or inhibition of an AAV vector transduction into human tissues, wherein the method comprises: (a) providing groups of non-human animals, Group A and Group B, wherein Group A comprises one or more a T-, B- and NK cell deficient or impaired in function non-human animals, and Group B comprises one or a T-, B- and NK cell deficient or impaired in function non-human animals, further comprising a deletion or mutation of Adeno-associated virus receptor (AAVR) resulting in a non-functional non-human animal AAVR, wherein the one or more a T-, B- and NK cell deficient or impaired in function allow re-populating with human tissue to establish human chimerism in the non-human animal; (b) transplanting one or more human tissues into the one or more non-human animals of both groups of (a); (c) infecting the non-human animals of both groups of (b) with an AAV vector; and (d) determining the level of transduction of the AAV vector into human tissue of the non-human animals of Group A and Group B.

In another aspect, provided herein is a method of determining AAVR-dependent or AAVR-independent modification and/or inhibition of an AAV vector transduction into one or more human tissues, wherein the method comprises: (a) providing two groups of non-human animals, Group A and Group B, wherein Group A comprises one or more IL-2Rg^{-/-}/Rag 2^{-/-} non-human animals, and Group B comprises one or more IL-2Rg^{-/-}/Rag 2^{-/-} non-human animals, further comprising a deletion or mutation of Adeno-associated virus receptor (AAVR) resulting in a non-functional non-human animal AAVR; (b) transplanting one or more human tissues into the one or more non-human animals of both groups of (a); (c) infecting the non-human animals of both groups of (b) with an AAV vector; and (d) determining the level of transduction of the AAV vector into human tissue of the non-human animals of Group A and Group B. In some embodiments, the non-human animal further comprises a Fah^{-/-}.

In the present disclosure, examples of the “chimeric non-human animal” include portions of the non-human animal. The term “a portion(s) of the chimeric non-human animal” refers to, non-human animal-derived tissues, body fluids, cells, and disrupted products thereof or extracts therefrom, for example (the examples thereof are not particularly limited to them). Examples of such tissues include, but are not particularly limited to, heart, lungs, kidney, liver, gallbladder, pancreas, spleen, intestine, muscle, blood vessel, brain, testis, ovary, uterus, placenta, marrow, thyroid gland, thymus gland, and mammary gland. Examples of body fluids include, but are not particularly limited to, blood, lymph fluids, and urine. The term “cells” refers to cells contained in the above tissues or body fluids, and examples thereof include cultured cells, sperm cells, ova, and fertilized eggs obtained by isolation or culture thereof. Examples of cultured cells include both primary cultured cells and cells of an established cell line. Examples of the portions of the chimeric non-human animal also include tissues, body fluids, and cells at the developmental stage (embryonic stage), as well as the disrupted products or extracts thereof. In addition, a cell line from the mice of the present disclosure can be established using a known method (Primary Culture Methods for Embryonic Cells (Shin Seikagaku Jikken Koza (New Biochemical Experimental Lecture Series), Vol. 18, pages 125-129, TOKYO KAGAKU DOZIN CO., LTD., and Manuals for. Mouse Embryo Manipulation, pages 262-264, Kindai Shuppan)).

The chimeric non-human animal of the present disclosure can be an immunodeficient chimeric non-human animal (e.g., a mouse). The immunodeficient chimeric non-human animal of the present disclosure can be used as a host mouse for transplantation of human hepatocytes. Examples of the “immunodeficient non-human animal” may be any chimeric non-human animal that does not exhibit rejection against hepatocytes from a different animal origin (in particular, human hepatocytes), and include, but are not limited to, SCID (severe combined immunodeficiency) mice exhibiting deficiency in T- and B-cell lines, NUDE mice that have lost T cell functions because of genetic deletion of the thymus gland, and RAG2 knockout mice produced by knocking out the RAG2 gene by a known gene targeting method (Science, 244: 1288-1292, 1989).

Moreover, the present disclosure provides a chimeric non-human animal having human hepatocytes. The chimeric non-human animal of the present disclosure can be immunologically deficient. The chimeric non-human animal of the present disclosure can be prepared by transplanting human hepatocytes into an immunodeficient chimeric non-human animal of the present disclosure.

As human hepatocytes to be used for transplantation, human hepatocytes isolated from normal human liver tissue by a conventional method such as a collagenase perfusion method can be used. The thus separated hepatocytes can also be used after cryopreservation. Alternatively, the chimeric non-human animal hepatocytes, which are defined as the human hepatocytes separated by a technique such as a collagenase perfusion method from a chimeric non-human animal liver, in which chimeric non-human animal hepatocytes have been replaced by human hepatocytes, can be used in a fresh state, and the cryopreserved chimeric non-human animal hepatocytes are also available after thawing.

Such human hepatocytes can be transplanted into the liver via the spleen of a chimeric non-human animal (e.g. mouse) of the present disclosure. Such human hepatocytes can also be directly transplanted via the portal vein. The number of human hepatocytes to be transplanted may range from about 1 to 2,000,000 cells and preferably range from about 200,000 to 1,000,000 cells. The gender of the chimeric non-human animal of the present disclosure is not particularly limited. Also, the age on days of the mouse of the present disclosure upon transplantation is not particularly limited. When human hepatocytes are transplanted into a young chimeric non-human animal (early weeks of age), human hepatocytes can more actively proliferate as the chimeric non-human animal grows. For example, about 0- to 40-day-old mice after birth, and particularly about 8- to 40-day-old mice after birth are preferably used.

The transplanted human hepatocytes account for any percentage of human chimerism of at least 5%, at least 10%, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99% or 100% of all hepatocytes in the chimeric liver of the chimeric non-human animal.

Described herein are non-human animals comprising one or more gene deletions. In general, a biallelic deletion of a gene is denoted “−/−”, e.g., “Fah−/−.”

A human nucleic sequence encoding an exemplary Il2-rg protein of the disclosure consist of or comprises Genbank Accession number: NM 000206.2:

| (SEQ ID NO: 1) | |
|---|---|
| 1 | agaggaaacg tgtgggtggg gaggggtagt gggtgaggga cccaggttcc tgacacagac |
| 61 | agactacacc cagggaatga agagcaagcg ccatgttgaa gccatcatta ccattcacat |
| 121 | ccctcttatt cctgcagctg cccctgctgg gagtggggct gaacacgaca attctgacgc |
| 181 | ccaatgggaa tgaagacacc acagctgatt tcttcctgac cactatgccc actgactccc |
| 241 | tcagtgtttc cactctgccc ctcccagagg ttcagtgttt tgtgttcaat gtcgagtaca |
| 301 | tgaattgcac ttggaacagc agctctgagc cccagcctac caacctcact ctgcattatt |
| 361 | ggtacaagaa ctcggataat gataaagtcc agaagtgcag ccactatcta ttctctgaag |
| 421 | aaatcacttc tggctgtcag ttgcaaaaaa aggagatcca cctctaccaa acatttgttg |
| 481 | ttcagctcca ggacccacgg gaacccagga gacaggccac acagatgcta aaactgcaga |
| 541 | atctggtgat cccctgggct ccagagaacc taacacttca caaactgagt gaatcccagc |
| 601 | tagaactgaa ctggaacaac agattcttga accactgttt ggagcacttg gtgcagtacc |
| 661 | ggactgactg ggaccacagc tggactgaac aatcagtgga ttatagacat aagttctcct |
| 721 | tgcctagtgt ggatgggcag aaacgctaca cgtttcgtgt tcggagccgc tttaacccac |
| 781 | tctgtggaag tgctcagcat tggagtgaat ggagccaccc aatccactgg gggagcaata |
| 841 | cttcaaaaga gaatcctttc ctgtttgcat tggaagccgt ggttatctct gttggctcca |
| 901 | tuggattgat tatcagcctt ctatgtgtgt atttctggct ggaacggacg atgccccgaa |
| 961 | ttcccaccct gaagaaccta gaggatcttg ttactgaata ccacgggaac ttttcggcct |
| 1021 | ggagtggtgt gtctaaggga ctggctgaga gtctgcagcc agactacagt gaacgactct |
| 1081 | gcctcgtcag tgagattccc ccaaaaggag gggcccttgg ggaggggcct ggggcctccc |
| 1141 | catgcaacca gcatagcccc tactgggccc ccccatgtta caccctaaag cctgaaacct |
| 1201 | gaaccccaat cctctgacag aagaacccca gggtcctgta gccctaagtg gtactaactt |
| 1261 | tccttcattc aacccacctg cgtctcatac tcacctcacc ccactgtggc tgatttggaa |
| 1321 | ttttgtgccc ccatgtaagc accccttcat ttggcattcc ccacttgaga attacccttt |
| 1381 | tgccccgaac atgtttttct tctccctcag tctggccctt ccttttcgca ggattcttcc |
| 1441 | tccctccctc tttccctccc ttcctctttc catctaccct ccgattgttc ctgaaccgat |
| 1501 | gagaaataaa gtttctgttg ataatcatca aaaaaaaaaa |

The corresponding human amino acid sequence of an exemplary Il2-rg protein of the disclosure consist of or comprises Genbank Accession number: NP-000197.1:

| (SEQ ID NO: 2) | |
|---|---|
| 1 | mlkpslpfts llflqlpllg vglnttiltp ngnedttadf flttmptdsl svstlplpev |
| 61 | qcfvfnveym nctwnsssep qptnltlhyw yknsdndkvq kcshylfsee itsgcqlqkk |
| 121 | eihlyqtfvv qlqdpreprr qatqmlklqn lvipwapenl tlhklsesql elnwnnrfln |
| 181 | hclehlvqyr tdwdhswteq svdyrhkfsl psvdgqkryt frvrsrfnpl cgsaqhwsew |
| 241 | shpihwgsnt skenpflfal eavvisvgsm gliisllcvy fwlertmpri ptlknledlv |
| 301 | teyhgnfsaw sgvskglaes lqpdyserlc lvseippkgg algegpgasp cnqhspywap |
| 361 | pcytlkpet |

A murine nucleic sequence encoding an exemplary Il2-rg protein of the disclosure consist of or comprises Genbank Accession number: NM_013563.4:

| (SEQ ID NO: 3) | |
|---|---|
| 1 | aggaaatgta tgggtgggga gggcttgtgg gagagtggtt cagggttctg acacagacta |
| 61 | cacccagaga aagaagagca agcaccatgt tgaaactatt attgtcacct agatccttct |
| 121 | tagtccttca gctgctcctg ctgagggcag ggtggagctc caaggtcctc atgtccagtg |
| 181 | cgaatgaaga catcaaagct gatttgatcc tgacttctac agcccctgaa cacctcagtg |
| 241 | ctcctactct gccccttcca gaggttcagt gctttgtgtt caacatagag tacatgaatt |
| 301 | gcacttggaa tagcagttct gagcctcagg caaccaacct cacgctgcac tataggtaca |
| 361 | aggtatctga taataataca ttccaggagt gcagtcacta tttgttctcc aaagagatta |
| 421 | cttctggctg tcagatacaa aaagaagata tccagctcta ccagacattt gttgtccagc |
| 481 | tccaggaccc ccagaaaccc cagaggcgag ctgtacagaa gctaaaccta cagaatcttg |
| 541 | tgatcccacg ggctccagaa aatctaacac tcagcaatct gagtgaatcc cagctagagc |
| 601 | tgagatggaa aagcagacat attaaagaac gctgtttaca atacttggtg cagtaccgga |
| 661 | gcaacagaga tcgaagctgg acggaactaa tagtgaatca tgaacctaga ttctccctgc |
| 721 | ctagtgtgga tgagctgaaa cggtacacat ttcgggttcg gagccgctat aacccaatct |
| 781 | gtggaagttc tcaacagtgg agtaaatgga gccagcctgt ccactggggg agtcatactg |
| 841 | tagaggagaa tccttccttg tttgcactgg aagctgtgct tatccctgtt ggcaccatgg |
| 901 | ggttgattat taccctgatc tttgtgtact gttggttgga acgaatgcct ccaattcccc |
| 961 | ccatcaagaa tctagaggat ctggttactg aataccaagg gaacttttcg gcctggagtg |
| 1021 | gtgtgtctaa agggctgact gagagtctgc agccagacta cagtgaacgg ttctgccacg |
| 1081 | tcagcgagat tccccccaaa ggaggggccc taggagaggg gcctggaggt tctccttgca |
| 1141 | gcctgcatag cccttactgg cctcccccat gttattctct gaagccggaa gcctgaacat |
| 1201 | caatcctttg atggaacctc aaagtcctat agtcctaagt gacgctaacc tcccctactc |
| 1261 | accttggcaa tctggatcca atgctcactg ccttcccttg gggctaagtt tcgatttcct |
| 1321 | gtcccatgta actgcttttc tgttccatat gccctacttg agagtgtccc ttgccctctt |
| 1381 | tccctgcaca agccctccca tgcccagcct aacacctttc cactttcttt gaagagagtc |
| 1441 | ttaccctgta gcccagggtg gctgggagct cactatgtag gccaggttgg cctccaactc |
| 1501 | acaggctatc ctcccacctc tgcctcataa gagttggggt tactggcatg caccaccaca |
| 1561 | cccagcatgg tccttctctt ttataggatt ctccctccct ttttctacct atgattcaac |
| 1621 | tgtttccaaa tcaacaagaa ataaagtttt taaccaatga tca |

The corresponding murine amino acid sequence of an exemplary Il2-rg protein of the disclosure consist of or comprises Genbank Accession number: NP_038591.1:

| (SEQ ID NO: 4) | |
|---|---|
| 1 | mlklllsprs flvlqllllr agwsskvlms sanedikadl iltstapehl saptlplpev |
| 61 | qcfvfnieym nctwnsssep qatnltlhyr ykvsdnntfq ecshylfske itsgcqiqke |
| 121 | diqlyqtfvv qlqdpqkpqr ravqklnlqn lviprapenl tlsnlsesql elrwksrhik |
| 181 | erclqylvqy rsnrdrswte livnheprfs lpsvdelkry tfrvrsrynp icgssqqwsk |
| 241 | wsqpvhwgsh tveenpslfa leavlipvgt mgliitlifv ycwlermppi ppiknledlv |
| 301 | teyqgnfsaw sgvskgltes lqpdyserfc hvseippkgg algegpggsp cslhspywpp |
| 361 | pcyslkpea |

A human nucleic sequence encoding an exemplary Rag2 protein of the disclosure consist of or comprises Genbank Accession number: NM_000536.3:

| (SEQ ID NO: 5) | |
|---|---|
| 1 | attagatcag tgttcataag aacatctgta ggcacacata cacactctct ttacagtcag |
| 61 | ccttctgctt gccacagtca tagtgggcag tcagtgaatc ttccccaagt gctgacaatt |
| 121 | aatacctggt ttagcggcaa agattcagag aggcgtgagc agcccctctg gccttcagac |
| 181 | aaaaatctac gtaccatcag aaactatgtc tctgcagatg gtaacagtca gtaataacat |
| 241 | agccttaatt cagccaggct tctcactgat gaattttgat ggacaagttt tcttctttgg |
| 301 | acaaaaaggc tggcccaaaa gatcctgccc cactggagtt ttccatctgg atgtaaagca |
| 361 | taaccatgtc aaactgaagc ctacaatttt ctctaaggat tcctgctacc tccctcctct |
| 421 | tcgctaccca gccacttgca cattcaaagg cagcttggag tctgaaaagc atcaatacat |
| 481 | catccatgga gggaaaacac caaacaatga ggtttcagat aagatttatg tcatgtctat |
| 541 | tgtttgcaag aacaacaaaa aggttacttt tcgctgcaca gagaaagact tggtaggaga |
| 601 | tgttcctgaa gccagatatg gtcattccat taatgtggtg tacagccgag ggaaaagtat |
| 661 | gggtgttctc tttggaggac gctcatacat gccttctacc cacagaacca cagaaaaatg |
| 721 | gaatagtgta gctgactgcc tgccctgtgt tttcctggtg gattttgaat ttgggtgtgc |
| 781 | tacatcatac attcttccag aacttcagga tgggctatct tttcatgtct ctattgccaa |
| 841 | aaatgacacc atctatattt taggaggaca ttcacttgcc aataatatcc ggcctgccaa |
| 901 | cctgtacaga ataagggttg atcttcccct gggtagccca gctgtgaatt gcacagtctt |
| 961 | gccaggagga atctctgtct ccagtgcaat cctgactcaa actaacaatg atgaatttgt |
| 1021 | tattgttggt ggctatcagc ttgaaaatca aaaaagaatg atctgcaaca tcatctcttt |
| 1081 | agaggacaac aagatagaaa ttcgtgagat ggagacccca gattggaccc cagacattaa |
| 1141 | gcacagcaag atatggtttg gaagcaacat gggaaatgga actgtttttc ttggcatacc |
| 1201 | aggagacaat aaacaagttg tttcagaagg attctatttc tatatgttga aatgtgctga |
| 1261 | agatgatact aatgaagagc agacaacatt cacaaacagt caaacatcaa cagaagatcc |
| 1321 | aggggattcc actccctttg aagactctga agaattttgt ttcagtgcag aagcaaatag |
| 1381 | ttttgatggt gatgatgaat ttgacaccta taatgaagat gatgaagaag atgagtctga |
| 1441 | gacaggctac tggattacat gctgccctac ttgtgatgtg gatatcaaca cttgggtacc |
| 1501 | attctattca actgagctca acaaacccgc catgatctac tgctctcatg gggatgggca |
| 1561 | ctgggtccat gctcagtgca tggatctggc agaacgcaca ctcatccatc tgtcagcagg |
| 1621 | aagcaacaag tattactgca atgagcatgt ggagatagca agagctctac acactcccca |
| 1681 | aagagtccta cccttaaaaa agcctccaat gaaatccctc cgtaaaaaag gttctggaaa |
| 1741 | aatcttgact cctgccaaga aatcctttct tagaaggttg tttgattagt tttgcaaaag |
| 1801 | cctttcagat tcaggtgtat ggaatttttg aatctatttt taaaatcata acattgattt |
| 1861 | taaaaataca tttttgttta tttaaaatgc ctatgttttc ttttagttac atgaattaag |
| 1921 | ggccagaaaa aagtgtttat aatgcaatga taaataaagt cattctagac cctatacatt |
| 1981 | ttgaaaatat tttacccaaa tactcaattt actaatttat tcttcactga ggatttctga |
| 2041 | tctgattttt tattcaacaa accttaaaca cccagaagca gtaataatca tcgaggtatg |
| 2101 | tttatattta ttatataagt cttggtaaca aataacctat aaagtgttta tgacaaattt |
| 2161 | agccaataaa gaaattaaca cccaaaagaa ttaaattgat tattttgtgc aacataacaa |
| 2221 | ttcggcagtt ggccaaaact taaaagcaag atctactaca tcccacatta gtgttcttta |
| 2281 | tataccttca agcaaccctt tcgattatgc ccatgaacaa gttagtttct catagcttta |
| 2341 | cagatgtaga tataaatata aatatatgta tacatataga tagataatgt tctccactga |
| 2401 | cacaaaagaa gaaataaata atctacatca aaaaaaaaaa aaaaaaaaaa aaaaaaa |

The corresponding human amino acid sequence of an exemplary Rag2 protein of the disclosure consist of or comprises Genbank Accession number: NP_000527.2:

| (SEQ ID NO: 6) | |
|---|---|
| 1 | mslqmvtvsn nialiqpgfs lmnfdgqvff fgqkgwpkrs cptgvfhldv khnhvklkpt |
| 61 | ifskdscylp plrypatctf kgslesekhq yiihggktpn nevsdkiyvm sivcknnkkv |
| 121 | tfrctekdlv gdvpearygh sinvvysrgk smgvlfggrs ympsthttte kwnsvadclp |
| 181 | cvflvdfefg catsyilpel qdglsfhvsi akndtiyilg ghslannirp anlyrirvdl |
| 241 | plgspavnct vlpggisvss ailtqtnnde fvivggyqle nqkrmicnii slednkieir |
| 301 | emetpdwtpd ikhskiwigs nmgngtvflg ipgdnkqvvs egfyfymlkc aeddtneeqt |
| 361 | tftnsqtste dpgdstpfed seefcfsaea nsfdgddefd tyneddeede setgywitcc |
| 421 | ptcdvdintw vpfystelnk pamiycshgd ghwvhaqcmd laertlihls agsnkyycne |
| 481 | hveiaralht pqrvlplkkp pmkslrkkgs gkiltpakks flrrlfd |

A murine nucleic sequence encoding an exemplary Rag2 protein of the disclosure consist of or comprises Genbank Accession number: NM_009020.3:

| (SEQ ID NO: 7) | |
|---|---|
| 1 | actctaccct gcagccttca gcttggcaca aactaaacag tgactcttcc ccaagtgccg |
| 61 | agtttaattc ctggcttggc cgaaaggatt cagagaggga taagcagccc ctctggcctt |
| 121 | cagtgccaaa ataagaaaga gtatttcaca tccacaagca ggaagtacac ttcatacctc |
| 181 | tctaagataa aagacctatt cacaatcaaa aatgtccctg cagatggtaa cagtgggtca |
| 241 | taacatagcc ttaattcaac caggcttctc acttatgaat tttgatggcc aagttttctt |
| 301 | ctttggccag aaaggctggc ctaagagatc ctgtcctact ggagtctttc attttgatat |
| 361 | aaaacaaaat catctcaaac tgaagcctgc aatcttctct aaagattcct gctacctccc |
| 421 | acctcttcgt tatccagcta cttgctcata caaaggcagc atagactctg acaagcatca |
| 481 | atatatcatt cacggaggga aaacaccaaa caatgagctt tccgataaga tttatatcat |
| 541 | gtctgtcgct tgcaagaata acaaaaaagt tactttccgt tgcacagaga aagacttagt |
| 601 | aggagatgtc cctgaaccca gatacggcca ttccattgac gtggtgtata gtcgagggaa |
| 661 | aagcatgggt gttctctttg gaggaccttc atacatgcct tctacccaga gaaccacaga |
| 721 | aaaatggaat agtgtagctg actgcctacc ccatgttttc ttgatagatt ttgaatttgg |
| 781 | gtgtgctaca tcatatattc tcccagaact tcaggatggg ctgtcttttc atgtttctat |
| 841 | tgccagaaac gataccgttt atattttggg aggacactca cttgccagta atatacgccc |
| 901 | tgctaacttg tatagaataa gagtggacct tcccctgggt accccagcag tgaattgcac |
| 961 | agtcttgcca ggaggaatct ctgtctccag tgcaatcctc actcaaacaa acaatgatga |
| 1021 | atttgttatt gtgggtggtt atcagctgga aaatcagaaa aggatggtct gcagccttgt |
| 1081 | ctctctaggg gacaacacga ttgaaatcag tgagatggag actcctgact ggacctcaga |
| 1141 | tattaagcat agcaaaatat ggtttggaag caacatggga aacgggacta ttttccttgg |
| 1201 | cataccagga gacaataagc aggctatgtc agaagcattc tatttctata ctttgagatg |
| 1261 | ctctgaagag gatttgagtg aagatcagaa aattgtctcc aacagtcaga catcaacaga |
| 1321 | agatcctggg gactccactc cctttgaaga ctcagaggaa ttttgtttca gtgctgaagc |
| 1381 | aaccagtttt gatggtgacg atgaatttga cacctacaat gaagatgatg aagatgacga |
| 1441 | gtctgtaacc ggctactgga taacatgttg ccctacttgt gatgttgaca tcaatacctg |
| 1501 | ggttccgttc tattcaacgg agctcaataa acccgccatg atctattgtt ctcatgggga |
| 1561 | tgggcactgg gtacatgccc agtgcatgga tttggaagaa cgcacactca tccacttgtc |
| 1621 | agaaggaagc aacaagtatt attgcaatga acatgtacag atagcaagag cattgcaaac |
| 1681 | tcccaaaaga aaccccccct tacaaaaacc tccaatgaaa tccctccaca aaaaaggctc |
| 1741 | tgggaaagtc ttgactcctg ccaagaaatc cttccttaga agactgtttg attaatttag |
| 1801 | caaaagcccc tcagactcag gtatattgct ctctgaatct actttcaatc ataaacatta |
| 1861 | ttttgatttt tgtttactga aatctctatg ttatgtttta gttatgtgaa ttaagtgctg |
| 1921 | ttgtgattta ttgttaagta taactattct aatgtgtgtt ttttaacatc ttatccagga |
| 1981 | atgtcttaaa tgagaaatgt tatacagttt tccattaagg atatcagtga taaagtatag |
| 2041 | aactcttaca ttattttgta acaatctaca tattgaatag taactaaata ccaataaata |
| 2101 | aactaatgca caaaaagtta agttcttttg tgtaataagt agcctatagt tcgtttaaac |
| 2161 | agttaaaacc aacagctata tcccacacta ctgctgttta taaattttaa ggtggcctct |
| 2221 | ggtttatact tatgagcaga attatatata ttggtcaata ccatgaagaa aaatttaatt |
| 2281 | ctatatcaag ccaggcatgg tgatggtgat acatgcctgt aatcctggca cttaggaagt |
| 2341 | ggaagaagga agtttgtgag tttgatgctt gttgaggtat gaccttttgc tatgtattgt |
| 2401 | agtgtatgag ccccaagacc tgcttgaccc agagacaaga gagtccacac atagatccaa |
| 2461 | gtaatgctat gtgaccttgc cccccggtta cttgtgatta ggtgaataaa gatgtcaaca |
| 2521 | gccaatagct gggcagaaga gccaaaagtg gggattgagg gtaccctggc ttgatgtagg |
| 2581 | aggagaccat gaggaaaggg gagaaaaaag tgatggagga ggagaaagat gccatgagct |
| 2641 | aggagttaag aaagcatggc catgagtgct ggccaattgg agttaagagc agcccagatg |
| 2701 | aaacatagta agtaataact cagggttatc gatagaaaat agattttagt gccgtactct |
| 2761 | ccccagccct agagctgact atggcttact gtaaatataa agtttgtatg tgtcttttat |
| 2821 | ccaggaacta aatggtcaaa ggtggagtag aaactctgga ttgggattaa atttttctac |
| 2881 | aacaaatgct ggcctgggct agattttatc tcatatccga aggctgacag aacacagagc |
| 2941 | actggtaaca ttgccacctg ccatgcacaa agacctgagt ctaatactgt ggacattttc |
| 3001 | ttgaagtatc tacatgtact tctggagtga aaacatattc caacaatatg cctttgttta |
| 3061 | aatcactcac tcactttggg ccctcacatt atatcctttc aaaatcaatg gttcacccct |
| 3121 | ttgaaaatgc ttagccatag tccctcatct tccttaaaga cagttgtcat ctctggaaat |
| 3181 | agtcacatgt cattcaaggt ccaatactgt gcagctctga agtatggcat taccacttta |
| 3241 | agtgaaaagt gaaatatgaa catgagctca gacaaaggtt tgggactatc actctcaagg |
| 3301 | aggctctact gctaagtcct gaactgcttt cacatgaata cagaaattat aacaaaaaat |
| 3361 | atgtaatcaa taaaaagaaa actttcatat tcc |

The corresponding murine amino acid sequence of an exemplary Rag2 gene of the disclosure consist of or comprises Genbank Accession number: NP_033046.1:

| (SEQ ID NO: 8) | |
|---|---|
| 1 | mslqmvtvgh nialiqpgfs lmnfdgqvff fgqkgwpkrs cptgvfhfdi kqnhlklkpa |
| 61 | ifskdscylp plrypatcsy kgsidsdkhq yiihggktpn nelsdkiyim svacknnkkv |
| 121 | tfrctekdlv gdvpeprygh sidvvysrgk smgvlfggrs ympstqrtte kwnsvadclp |
| 181 | hvflidfefg catsyilpel qdglsfhvsi arndtvyilg ghslasnirp anlyrirvdl |
| 241 | plgtpavnct vlpggisvss ailtqtnnde fvivggyqle nqkrmvcslv slgdntieis |
| 301 | emetpdwtsd ikhskiwfgs nmgngtiflg ipgdnkqams eafyfytlrc seedlsedqk |
| 361 | ivsnsqtste dpgdstpfed seefcfsaea tsfdgddefd tyneddedde svtgywitcc |
| 421 | ptcdvdintw vpfystelnk pamiycshgd ghwvhaqcmd leertlihls egsnkyycne |
| 481 | hvqiaralqt pkrnpplqkp pmkslhkkgs gkvltpakks flrrlfd |

A human nucleic sequence encoding an exemplary Fah protein of the disclosure consist of or comprises Genbank Accession number: NM_000137.2:

| (SEQ ID NO: 9) | |
|---|---|
| 1 | gagaccaaaa gtcaggtagg agcctccggg gtccctgctg tgtcacccgg acaggccgtg |
| 61 | ggggcgggca ggggggcggg gccgggcctg accacagcgg ccgagttcag tcctgctctc |
| 121 | cgcacgccac cttaggcccg cagccgtgcc gggtgctctt cagcatgtcc ttcatcccgg |
| 181 | tggccgagga ttccgacttc cccatccaca acctgcccta cggcgtcttc tcgaccagag |
| 241 | gcgacccaag accgaggata ggtgtggcca ttggcgacca gatcctggac ctcagcatca |
| 301 | tcaagcacct ctttactggt cctgtcctct ccaaacacca ggatgtcttc aatcagccta |
| 361 | cactcaacag cttcatgggc ctgggtcagg ctgcctggaa ggaggcgaga gtgttcttgc |
| 421 | agaacttgct gtctgtgagc caagccaggc tcagagatga caccgaactt cggaagtgtg |
| 481 | cattcatctc ccaggcttct gccacgatgc accttccagc caccatagga gactacacag |
| 541 | acttctattc ctctcggcag catgctacca acgtcggaat catgttcagg gacaaggaga |
| 601 | atgcgttgat gccaaattgg ctgcacttac cagtgggcta ccatggccgt gcctcctctg |
| 661 | tcgtggtgtc tggcacccca atccgaaggc ccatgggaca gatgaaacct gatgactcta |
| 721 | agcctcccgt atatggtgcc tgcaagctct tygacatgga gctggaaatg gctttttttg |
| 781 | taggccctgg aaacagattg ggagagccga tccccatttc caaggcccat gagcacattt |
| 841 | ttggaatggt ccttatgaac gactggagtg cacgagacat tcagaagtgg gagtatgtcc |
| 901 | ctctcgggcc attccttggg aagagttttg ggaccactgt ctctccgtgg gtggtgccca |
| 961 | tggatgctct catgcccttt gctgtgccca acccgaagca ggaccccagg cccctgccgt |
| 1021 | atctgtgcca tgacgagccc tacacatttg acatcaacct ctctgttaac ctgaaaggag |
| 1081 | aaggaatgag ccaggcggct accatatgca agtccaattt taagtacatg tactggacga |
| 1141 | tgctgcagca gctcactcac cactctgtca acggctgcaa cctgcggccg ggggacctcc |
| 1201 | tggcttctgg gaccatcagc gggccggagc cagaaaactt cggctccatg ttggaactgt |
| 1261 | cgtggaaggg aacgaagccc atagacctgg ggaatggtca gaccaggaag tttctgctgg |
| 1321 | acggggatga agtcatcata acagggtact gccaggggga tggttaccgc atcggctttg |
| 1381 | gccagtgtgc tggaaaagtg ctgcctgctc tcctgccatc atgagatttt ctctgctctt |
| 1441 | ctggaaacaa agggctcaag cacccctttc aaccctgtga ctggggtcct ccctcgggct |
| 1501 | gtaggcctgg tccgccattc agtgacaaat aaagccattg tgctctgagg cctgcactgc |
| 1561 | cgcagatgca gctgtgtcca cttatgatcg tgatttgatc cagtgggtca aggtgtgtaa |
| 1621 | agcctccctg ccagatattc attaatatgt tttctcactc ttattagtga ggtcaggggt |
| 1681 | ctttgtggga ttttcttatt agacatccca ggcctcctgg tattccatgg aatttgaaaa |
| 1741 | gagactggca cctgtagtag tcagggctct ccagagaaat agaaccaagg agaaagaaaa |
| 1801 | aaaaaaaaaa |

The corresponding human amino acid sequence of an exemplary Fah protein of the disclosure consist of or comprises Genbank Accession number: NP_000128.1:

| (SEQ ID NO: 10) | |
|---|---|
| 1 | msfipvaeds dfpihnlpyg vfstrgdprp rigvaigdqi ldlsiikhlf tgpvlskhqd |
| 61 | vfnqptlnsf mglgqaawke arvflqnlls vsqarlrddt elrkcafisq asatmhlpat |
| 121 | igdytdfyss rqhatnvgim frdkenalmp nwlhlpvgyh grassvvvsg tpirrpngqm |
| 181 | kpddskppvy gacklldmel emaffvgpgn rlgepipisk ahehifgmvl mndwsardiq |
| 241 | kweyvplgpf lgksfgttvs pwvvpmdalm pfavpnpkqd prplpylchd epytfdinls |
| 301 | vnlkgegmsq aaticksnfk ymywtmlqql thhsvngcnl rpgdllasgt isgpepenfg |
| 361 | smlelswkgt kpidlgngqt rkflldgdev iitgycqgdg yrigfgqcag kvlpallps |

The corresponding mouse nucleic acid sequence encoding an exemplary Fah protein of the disclosure consist of or comprises Genbank Accession number: NM_010176.4;

| (SEQ ID NO: 11) | |
|---|---|
| 1 | gggtgctaaa agaatcacta gggtggggag gcggtcccag tggggcgggt aggggtgtgt |
| 61 | gccaggtggt accgggtatt ggctggagga agggcagccc ggggttcggg gcggtccctg |
| 121 | aatctaaagg ccctcggcta gtctgatcct tgccctaagc atagtcccgt tagccaaccc |
| 181 | cctacccgcc gtgggctctg ctgcccggtg ctcgtcagca tgtcctttat tccagtggcc |
| 241 | gaggactccg actttcccat ccaaaacctg ccctatggtg ttttctccac tcaaagcaac |
| 301 | ccaaagccac ggattggtgt agccatcggt gaccagatct tggacctgag tgtcattaaa |
| 361 | cacctcttta ccggacctgc cctttccasa catcaacatg tcttcgatga gacaactctc |
| 421 | aataacttca tgggtctggg tcaagctgca tggaaggagg caagagcatc cttacagaac |
| 481 | ttactgtctg ccagccaagc ccggctcaga gatgacaagg agcttcggca gcgtgcattc |
| 541 | acctcccagg cttctgcgac aatgcacctt cctgctacca taggagacta cacggacttc |
| 601 | tactcttctc ggcagcatgc caccaatgtt ggcattatgt tcagaggcaa ggagaatgcg |
| 661 | ctgttgccaa attggctcca cttacctgtg ggataccatg gccgagcttc ctccattgtg |
| 721 | gtatctggaa ccccgattcg aagacccatg gggcagatga gacctgataa ctcaaagcct |
| 781 | cctgtgtatg gtgcctgcag actcttagac atggagttgg aaatggcttt cttcgtaggc |
| 841 | cctgggaaca gattcggaga gccaatcccc atttccaaag cccatgaaca cattttcggg |
| 901 | atggtcctca tgaacgactg gagcgcacga gacatccagc aatgggagta cgtcccactt |
| 961 | gggccattcc tggggaaaag ctttggaacc acaatctccc cgtgggtggt gcctatggat |
| 1021 | gccctcatgc cctttgtggt gccaaaccca aagcaggacc ccaagccctt gccatatctc |
| 1081 | tgccacagcc agccctacac atttgatatc aacctgtctg tctctttgaa aggagaagga |
| 1141 | atgagccagg cggctaccat ctgcaggtct aactttaagc acatgtactg gaccatgctg |
| 1201 | cagcaactca cacaccactc tgttaatgga tgcaacctga gacctgggga cctcttggct |
| 1261 | tctggaacca tcagtggatc agaccctgaa agctttggct ccatgctgga actgtcctgg |
| 1321 | aagggaacaa aggccatcga tgtggagcag gggcagacca ggaccttcct gctggacggc |
| 1381 | gatgaagtca tcataacagg tcactgccag ggggacggct accgtgttgg ctttggccag |
| 1441 | tgtgctggga aagtgctgcc tgccctttca ccagcctgaa gctccggaag tcacaagaca |
| 1501 | cacccttgcc ttatgaggat catgctacca ctgcatcagt caggaatgaa taaagctact |
| 1561 | ttgattgtgg gaaatgccac agaaaaaaaa aaaaaaa |

The corresponding murine amino acid sequence encoding an exemplary Fab protein of the disclosure consist of or comprises Genbank Accession number: NP_034306.2:

| (SEQ ID NO: 12) | |
|---|---|
| 1 | msfipvaeds dfpiqnlpyg vfstqsnpkp rigvaigdqi ldlsvikhlf tgpalskhqh |
| 61 | vfdettlnnf mglgqaawke araslqnlls asqarlrddk elrqraftsq asatmhlpat |
| 121 | igdytdfyss rqhatnvgim frgkenallp nwlhlpvgyh grassivvsg tpirrpmgqm |
| 181 | rpdnskppvy gacrlldmel emaffvgpgn rfgepipisk ahehifgmvl mndwsardiq |
| 241 | qweyvplgpf lgksfgttis pwvvpmdalm pfvvpnpkqd pkplpylchs qpytfdinls |
| 301 | vslkgegmsq aaticrsnfk hmywtmlqql thhsvngcnl rpgdllasgt isgsdpesfg |
| 361 | smlelswkgt kaidveqgqt rtflldgdev iitghcqgdg yrvgfgqcag kvlpalspa |

The adeno-associated virus (AAV) receptor (AAVR) disclosed herein, also referred to as Dyslexia-associated protein KIAA0319-like (KIAA0319L) protein, is a predicted type I transmembrane protein with five Ig-like domains in its ectodomain, referred to as polycystic kidney disease (PKD) domains. Ig-like domains mediate cell-cell adhesion and are present in various well-characterized virus receptors, including those for poliovirus, measles virus and reovirus. (Pillay S. et al., Nature 2016; 530:108-112) The PKD of AAVR have been shown to bind directly to the spike region of the AAV2 capsid adjacent to the icosahedral three-fold axis. (Zhang R. et al., Nat Microbiol, 2019 April; 4(4):675-682).

A murine amino acid sequence of an exemplary AAVR of the disclosure consist of or comprises Genbank Accession number: Q81K135:

| (SEQ ID NO: 13) | |
|---|---|
| 1 | mekrlgvkps paswvlpgyc wqtsvklprs lyllysffcf svlwlstdad esrcqqgktl |
| 61 | ygaglrtege nhlrllagsl pfhacraacc rdsachalww legmcfqadc skpqscqpfr |
| 121 | tdssnsmlii fqksqttddl gllpeddeph llrlgwgrts wrrqsllgap ltlsvpsshh |
| 181 | qsllrdrqkr dlsvvpthga mqhskvnhse eagalsptsa evrktitvag sftsnhttqt |
| 241 | pewpknvsih pepsehsspv sgtpqvkste hsptdaplpv apsysyatpt pqassqstsa |
| 301 | phpvvkelvv sagksvqitl pknevqlnaf vlpeaepget ytydwqlith ptdysgever |
| 361 | khsqslqlsk ltpglyefkv tvdgqnahge gyvnvtvkpe prknrppvav vspqfqeisl |
| 421 | pttstiidgs qstdddkivq yhweelkgpl reekisedta ilklsklvpg nytfsltvvd |
| 481 | sdgatnstta sltvnkavdy ppvanagpnq vitlpqnsit lfgnqstddh gitsyewsls |
| 541 | psskgkvvem qgvrtpalql samqegdyty qltvtdtagq qataqvtviv qpennkppqa |
| 601 | dagpdkeltl pvdsttldgs kstddqrvvs ylweqsrgpd gvqlenanss vatvtglqvg |
| 661 | tyvftltvkd ernlqsqssv nvivkeeink ppvakiagnv vvtlptstae ldgsrssddk |
| 721 | givsylwtrd etspaagevl nhsdhhpvlf lsnlvegtyt fhlkvtdakg esdtdrttve |
| 781 | vkpdprksnl veiildvnvs glterlkgml irqigvllgv ldsdiivqki qpyteqstkm |
| 841 | lffvqndpph qlfkghevaa mlkselqkqk adflifrale istvtcqlnc sdhghcdsft |
| 901 | krcvcdpfwm enfikvqlrd gdsncewsvl yviiasfviv valgilswtt iccckrqkgk |
| 961 | pkrksrykil datdqeslel kptsragska kgptlssslm hseseldsdd aiftwpdrek |
| 1021 | gkllygqngs vpngqtplks rsareeil |

A human amino acid sequence of an exemplary AAVR protein of the disclosure consist of or comprises Genbank Accession number: NP 079150:

| (SEQ ID NO: 14) | |
|---|---|
| 1 | mekrlgvkpn paswilsgyy wqtsakwlrs lylfytcfcf svlwlstdas esrcqqgktq |
| 61 | fgvglrsgge nhlwllegtp slqscwaacc qdsachvfww legmciqadc srpqscrafr |
| 121 | thssnsmlvf lkkfqtaddl gflpeddvph llglgwnwas wrqsppraal rpavsssdqq |
| 181 | slirklqkrg spsdvvtpiv tqhskvndsn elgglttsgs aevhkaitis splttdltae |
| 241 | lsggpknvsv qpeiseglat tpstqqvkss ektqiavpqp vapsysyatp tpqasfqsts |
| 301 | apypvikelv vsagesvqit lpknevqlna yvlqeppkge tytydwqlit hprdysgeme |
| 361 | gkhsqilkls kltpglyefk vivegqnahg egyvnvtvkp eprknrppia ivspqfqeis |
| 421 | lpttstvidg sqstdddkiv qyhweelkgp lreekisedt ailklsklvp gnytfsltvv |
| 481 | dsdgatnstt anltvnkavd yppvanagpn qvitlpqnsi tlfgnqstdd hgitsyewsl |
| 541 | spsskgkvve mqgvrtptlq lsamqegdyt yqltvtdtig qqataqvtvi vqpennkppq |
| 601 | adagpdkelt lpvdsttldg skssddqkii sylwektqgp dgvqlenans svatvtglqv |
| 661 | gtyvftltvk dernlqsqss vnvivkeein kppiakitgn vvitlptsta eldgskssdd |
| 721 | kgivsylwtr degspaagev lnhsdhhpil flsnlvegty tfhlkvtdak gesdtdrttv |
| 781 | evkpdprknn lveiildinv sqlterlkgm firqigvllg vldsdiivqk iqpyteqstk |
| 841 | mvffvqnepp hqifkgheva amlkselrkq kadflifral evntvtcqln csdhghcdsf |
| 901 | tkrcicdpfw menfikvqlr dgdsncewsv lyviiatfvi vvalgilswt viccckrqkg |
| 961 | kpkrkskyki ldatdqesle lkptsragik qkglllsssl mhseseldsd daiftwpdre |
| 1021 | kgkllhgqng svpngqtplk arspreeil |

The following examples are provided to better illustrate the claimed disclosure and are not to be interpreted as limiting the scope of the disclosure. To the extent that specific materials are mentioned, it is merely for purposes of illustration and is not intended to limit the disclosure. One skilled in the art may develop equivalent means or reactants without the exercise of inventive capacity and without departing from the scope of the disclosure.

### EXAMPLES

### Example 1: Generation of Il2rg^{-/-}/Rag2^{-/-}/Fah^{-/-} Mouse Model

The use of humanized chimeric mice models for studying and evaluating the use of AAV vectors for gene therapy, because the AAV vector preferably transduces mouse cells. The mouse AAV receptor is key for transduction with AAV vectors serotypes. Described herein are studies describing the generation a transgene free Il2rg^{-/-}/Rag2^{-/-}/Fah^{-/-} mice model with a mouse adeno-associated virus receptor knock out (AAVR KO) gene deletion, and use of the same to determine transduction efficiency of AAV vectors. There are many potential AAV serotypes that can be good clinical candidates for gene therapy in the liver, nevertheless, transduction efficiency even in the liver remains an important point. The immune response against the viral capsid is directly proportional to the dose of injected AAV (Nathwani A C et al., N Engl J Med 2014; 371:1994-2004.). Hence it is important to use as little AAV as possible to reduce the immune response, but sufficient AAV to get an efficient transduction and therapeutic effect. The present disclosure shows that human liver chimeric mouse in the background of AAVR deletion of the mouse (AAVR knockout mice) should be more specific and valuable for validation of human directed gene therapy, e.g. validation of gene therapy vectors in the human context. The human liver chimeric mouse with deleted AAV receptor of the murine tissue of the present disclosure should be useful to identify and evaluate the transduction efficiency of the most suitable and clinically translatable AAV gene therapy vectors.

### Materials and Methods

### Generation of AAVR-KO Strain by CRISPR/Cas9-Mediated Exonic Deletion.

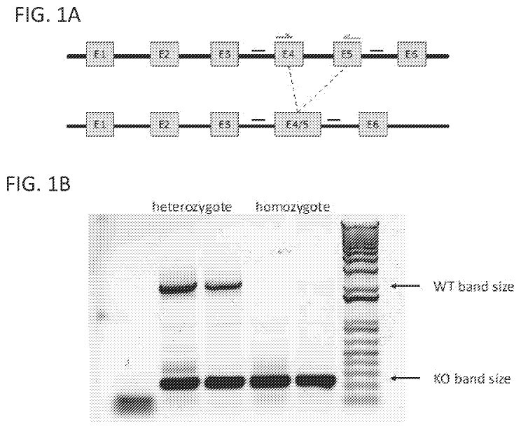

The design and cloning of sgRNA for generating the AAVR KO knock out mouse disclosed herein was done as described previously in the art (Johnson C G et al., Curr Protoc Mol Biol 2020; 130:e117). Exons 4 and 5 of the mouse AAVR gene were targeted (FIG. 1A). Provided below are the sgRNA sequences used for the study described herein:

sgRNA Ex4
(SEQ ID NO: 15)
ACTTGCGGAGTACCAGATAC.AGG
sgRNA Ex5
(SEQ ID NO: 16)
CATTCTTAGGCAGGGTGATC.TGG

The sgRNAs and Cas9 were in vitro transcribed using MEGAshortscript T7 Transcription Kit (Life tech, AM1354) and mMessage mMachine T7 ULTRA Kit (life tech AM1345), respectively. A mix of 15 ng/μL of each sgRNA and 60 ng/μL of Cas9 mRNA in 1×PBS was used for the zygote's microinjection. Cas9 and sgRNA were injected into homozygous zygotes from TIRF (transgene free Il2rg−/−/Rag2−/−/Fah−/−) mice.

Nineteen founder pups were obtained, four of which died after birth. All of the pups were screened for deletions. Seven out of the fifteen mice that survived were heterozygotes for a deletion. A total of 4 deletions were obtained, some of them shared present in more than one mouse, and all of them lead to a premature stop codon after the junction site.

Positive animals were backcrossed twice with the TIRF strain to eliminate any possible off-target mutation. Heterozygote F2 mice were crossed to each other to obtain homozygote pups. Homozygous mice were normal in size and did not show any abnormal phenotype, but seemed to have a fertility problem and a homozygous colony could not be established.

### TIRFA Strain Genotyping

All of the homozygous pups obtained were screened by PCR using the following primers:

AAVR Ex4 For
(SEQ ID NO: 17)
5′ACAGTTGCCGGTTCCTTCAC 3′
AAVR_In5 Rev
(SEQ ID NO: 18)
5′ CCACATGCACATCACAACCTC 3′

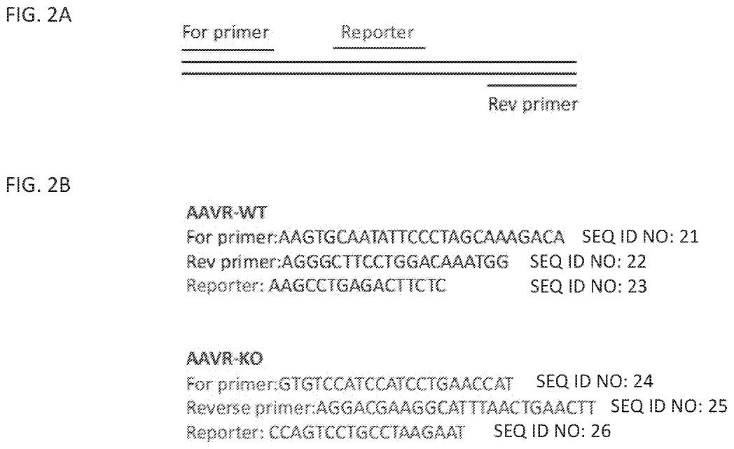

The expected PCR bands for wild type and knocked out mice were 2.3 Kb and 200 bp, respectively (FIG. 1B). PCR bands corresponding to deleted alleles were purified and sent for Sanger sequencing. Once the junction was defined, the further offspring was genotyped by Transnetyx Incorporation. by semi-quantitative PCR using Taqman probes (FIG. 2A-2B). The Taqman probes are single stranded DNA probes that have a fluorophore covalently attached to the 5′ and a quencher to the 3′ end. The quencher absorbs the fluorescence while in close proximity to the fluorophore. The probes bind to the genomic region of interest that is amplified by a Taq polymerase using specific primers. Once the strands are separated for each amplification step, the probe can bind to the strand to which it has affinity for. Degradation of the annealed probe occurs by the 5′ to 3′ exonuclease activity of the Taq polymerase in the next amplification round. The fluorophore is released, and a quantitative PCR thermal cycler detects the emitted fluorescence, which accumulates over the cycles.

Transplantation of Human Hepatocytes into TIRFA Strain

Human cryopreserved hepatocytes (Lonza) were transplanted into the TIRFA strain as previously described (Bissig-Choisat B, et al., Nature communications 2015; 6:7339; Barzi M et al., Nature communications 2017; 8:39). In brief, the abdominal cavity was opened by a midabdominal incision, and 3×10⁶ human hepatocytes in a volume of 100 μl PBS were injected into the spleen. Immediately after transplantation, selection pressure towards transplanted human hepatocytes was applied by withdrawing the drug NTBC from the drinking water in the following steps: 2 days at 25%, then 2 days at 12% and eventually 2 days at 6% of the colony maintenance dose (100%=7.5 mg/A) prior to discontinuing the drug completely (Bissig K D et al., The Journal of clinical investigation 2010). In order to determine the extent of human chimerism, human albumin (ELISA, Bethyl laboratories) was measured in the murine blood, because human albumin levels correlate with the level of human chimerism assessed by immunostaining of human hepatocytes (Bissig K D et al., The Journal of clinical investigation 2010). Only mice with a human chimerism >70% were used for AAV injections

### AAV Production

A triple-plasmid transfection protocol was used to generate rAAV vectors (Shen S et al., J Biol Chem 2013; 288.28814-28823); the transfection mixture contained. (1) the pXR helper plasmid; (2) the adenoviral helper plasmid pXX6-80; and (3) the dTomato, driven by a CMV promoter, flanked by AAV2 ITRs. Vector purification was carried out using iodixanol gradient ultracentrifugation followed by desalting with ZebaSpin desalting columns (40K MWCO; ThermoScientific, Waltham, MA, USA). vg titers were obtained by qPCR (LightCycler 480; Roche Applied Sciences, Pleasanton, CA, USA) using primers designed to selectively bind AAV2 ITRs (forward, 50-AACATGCTACGCAGAGAGGGAGTGG-30 (SEQ ID NO: 19); reverse, 50-CATGAGACAAGGAACCCCTAGTGATGGAG-30 (SEQ ID NO: 20)).

Immunostaining was performed on formalin-fixed paraffin-embedded liver. Paraffin sections (5 μm think) were dewaxed and antigen retrieval was performed in citrate buffer pH 6.0. Immunostaining was performed after blocking with 1% donkey serum+0.2% Triton with rabbit antibody against hFAH (Sigma Aldrich). After washing in PBS, the sections were stained with fluorescent-labeled secondary antibodies (Jackson Immunoresearch Laboratories) in 1% donkey serum for 30 min, washed again, and mounted with Vectashield plus DAPI (Vector Labs).

### Example 2: Transduction Efficiency of Human and Mouse Hepatocytes of Human Liver Chimeric Mice with Different AAV Serotypes

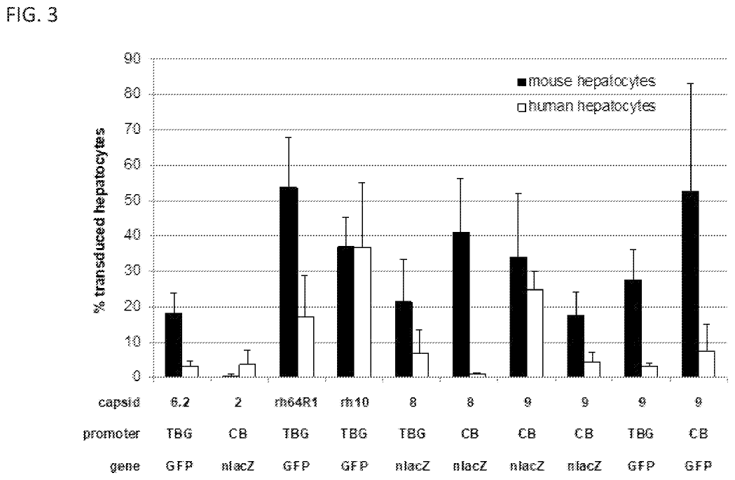

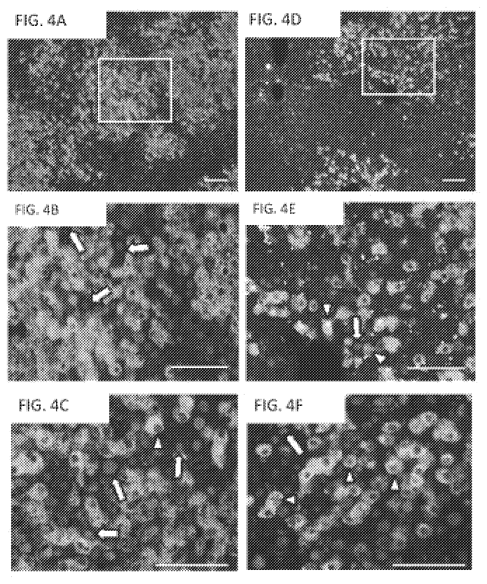

Human liver chimeric mice (mice with humanized livers) have been used to determine AAV transduction efficiencies of human hepatocytes (Bissig-Choisat B et al., Nature communications 2015; Lisowski L et al., Nature 2014) in vivo since extrapolating results from animal studies to humans for gene therapy is problematic. There are potential differences in uptake, delivery to the nucleus, uncoating, second strand synthesis of the recombinant genome, and persistence and expression of the transgene, that must be considered. Therefore, chimeric humanized liver mice provide a unique in vivo platform to further evaluate candidate AAV serotypes for transduction efficiency of human hepatocytes (Bissig-Choisat B et al., Nature communications 2015; Lisowski L et al., Nature 2014), overcoming some of these limitations. In the study described herein FRG (Fah−/−/Rag2−/−/Il2rg−/−) mice were repopulated with healthy human hepatocytes that were transduced with different AAV serotypes. Each transduced AAV vectors expressed different expression cassettes. The transduction efficiency was evaluated in terms of the percentage of AAV transduced human and mouse hepatocytes in the FRG human chimeric liver mice by counting human cells immunostained for FAH or human nuclear staining, and transduced cells (positive for LacZ or GFP). The result of the study described herein shows that many AAV serotypes validated for transduction efficiency in humanized mice transduce murine hepatocytes much better than human hepatocytes (FIG. 3). Most importantly, two clinically used serotypes, AAV8 and AAV9 also seem to transduce murine hepatocytes much more readily than human hepatocytes (FIGS. 4B-4F) (Bissig-Choisat B et al., Nature communications 2015). This “squelching” or “sink” effect of murine hepatocytes reduces the value of human liver chimeric mouse systems for evaluation of transduction efficiencies of AAV vectors for human hepatocytes since less total AAV particles are available to bind to the human cells. The present study investigates the mechanism of this “squelching” or “sink” effect of murine hepatocytes on the AAV transduction efficiency into human hepatocytes, and methods of reducing the same.

### Example 3: Generation of TIRFA Mouse Model

Recently, a AAV receptor (AAVR) has been identified (Pillay S et al., Nature 2016). In the study described herein, it was hypothesized that an AAVR knock out human liver chimeric mice would be a good model for validation of gene therapy vectors in the human context. The mouse AAVR gene was deleted using a CRISPR approach in zygotes of TIRF mice (transgene free Il2rg−/−/Rag2−/−/Fah−/−) (JHEP reference March 2021) using the methods and materials described herein. These TIRFA mice generated using the methods described herein were viable and had no obvious pathology when heterozygous or homozygous for the AAVR gene KO. Initial attempts to breed the homozygous TIRFA mice described herein were not successful and the studies described herein were done using pups from heterozygous breeding pairs. TIRF(A) mice (homozygous, heterozygous and wild-type for AAVR) described herein were humanized with human hepatocytes as described previously (Bissig-Choisat B et al., Nature communications 2015; Lisowski L et al., Nature 2014). Humanization in TIRFA animals were assessed by measurements of human albumin in the murine blood using a human specific albumin test using the methods described herein. Human albumin was detected between 1 mg/mL and 5 mg/ml serum, which is compatible with high human liver chimerism (Bissig K D et al., The Journal of clinical investigation 2010). The results disclosed herein show that the present disclosure provides an immunodepleted chimeric mice model that is deficient or depleted in endogenous AAVR and that allows that allows re-populating with human hepatocytes to establish human chimerism in the liver of the mice.

### Example 4: Evaluation of Transduction Efficiencies of AAV Serotypes in Human Hepatocytes in Humanized TIRFA Livers

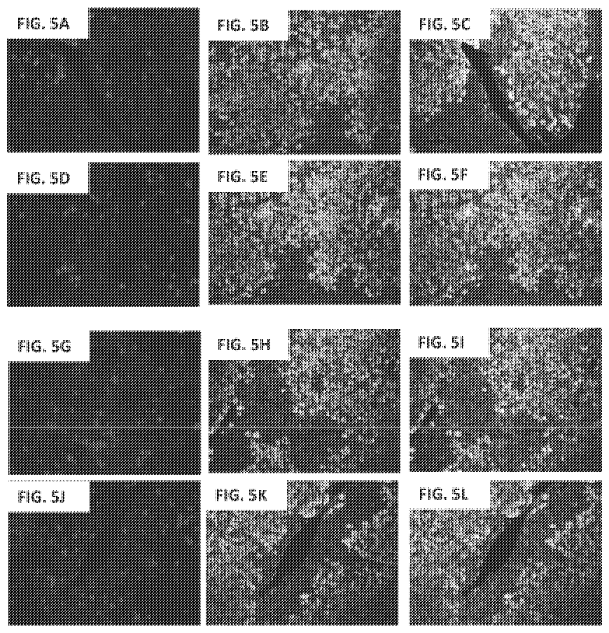

In the study described herein, the transduction efficiencies of AAV serotypes in human hepatocytes in the humanized TIRFA livers was evaluated. As described in Example 2, a AAV serotype 9 (AAV9) gene therapy vector was produced, containing an expression cassette of dTomato under an ubiquitous promoter (hybrid CMV enhancer/chicken β-actin (CBA)). All mice were injected into the tail vein with 1×10² vg/mouse, a dose, which transduces the whole murine liver in the presence of the AAVR. One months after injection humanized mice were euthanized and livers harvested. In the study described herein, efficient humanization was determined by (immunostaining of FAH), and transduction of human hepatocytes by AAV9 was determined by dTomato immunostaining (FIGS. 5C, 5F, 5I and 5L). The results described herein show that AAV9 efficiently transduced human cells and only very little murine cells in humanized TIRFA mice with homozygous deletion of the AAVR, while the majority of transduced cells in humanized TIRFA mice with heterozygous deletion of the AAVR (AAVR+/−) were the murine hepatocytes, similar to humanized TIRF or FRG, which are both AAVR+/+ wild type (FIG. 4D).

Based on the experimental data disclosed in the above stated examples, the “squelching” or “sink” effect of murine hepatocytes on transduction with AAV vectors in a chimeric non-human animal model can be reduced or inhibited and transduction of human hepatocytes can be achieved by knocking out the non-human animal AAVR. The examples disclosed herein show that the chimeric non-human animal model disclosed herein can be used for determining the biology and efficiency of AAV transduction into human cells.

### Example 5: Generation of Teratoma from Human iPS Cells in TIRFA Mice

Most AAV serotypes and recombinant capsids have high tropism for the liver and when injecting AAV intravenously the vast majority of AAVs transduce hepatocytes. While this is beneficial for many liver-directed gene therapy approaches, some are intended to target other organs like muscle or the brain. Hence, the liver can act as a sponge (squelching effect) so that very few AAV transduce the target tissue. To evaluate if the tropism of a specific capsid is higher for any given organ than the one for liver, humanized mice need to have liver tissue and the other human tissue in the same mouse. Hence if the tropism of a capsid is much higher for liver, than say muscle, then this specific capsid is not suitable for transduction of muscle in patients. To have a dual or multiple organ systems in a human liver TIRFA mouse, teratoma from induced pluripotent stem (iPS) cells were generated. Teratoma have fully differentiated and functional tissue from all three germ layers (Endo-, Ecto- and Mesoderm).

### Methods:

Generation of Teratoma from Human iPS Cells

Human induced pluripotent (iPS) cells were cultured on Matrigel using standard iPS cell media (mTeSR from StemCells Technologies). When reaching 80% confluency on a 10-cm dish, cells were gently scrapped off the plate (cell scraper) in media to form cell clumps and spun down at 500 g for 5 min. Cell clumps (˜1×10⁷ cells) are resuspended in 50-100 μl of fresh media and injected subcutaneously into a human liver chimeric or non-humanized TIRFA mouse.

### Immunostaining Chimeric Livers and Teratomas:

Immunostaining was performed on formalin-fixed paraffin-embedded liver. Paraffin sections (5 mm thick) were dewaxed and antigen retrieval was performed in citrate buffer pH 6.0. Immunostaining was performed after blocking with 1% donkey serum or with the corresponding reagent (for immunohistochemistry) following by incubation with either mouse antibody against hLDH (Santa Cruz, diluted 1:100) and rabbit anti-RFP antibody (Rockland, diluted 1:100) for liver sections or with anti-GFP (Abcam, diluted 1:200) for teratomas. After washing in PBS, the sections were stained with either fluorescent-labeled secondary antibodies (Jackson Immunoresearch Laboratories) or with the corresponding secondary antibodies from ImpressDuet and developed following the manufacturer's instructions (Vector Labs).

Humanized liver mice (human liver chimeric mice) were generated by repopulating the liver of TIRFA mice as explained above. After generation of high human liver chimerism (>20% human tissue), human induced pluripotent stem (iPS) cells were injected subcutaneously. 3-4 months after injection mice formed clearly visible subcutaneous teratoma. 1-3 weeks after formation of teratoma, AAV9 carrying a ubiquitous GFP expression cassette was injected intravenously.

### Results

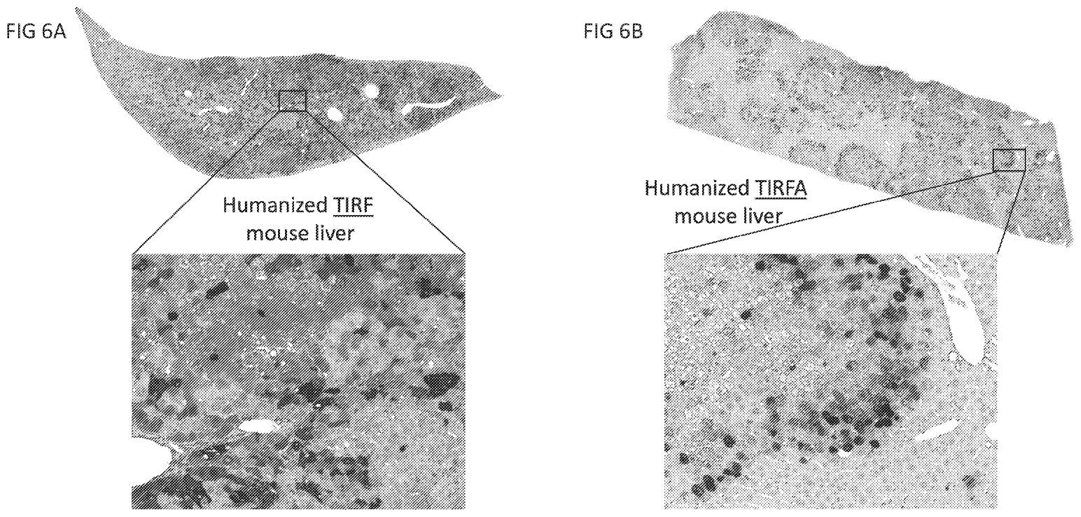

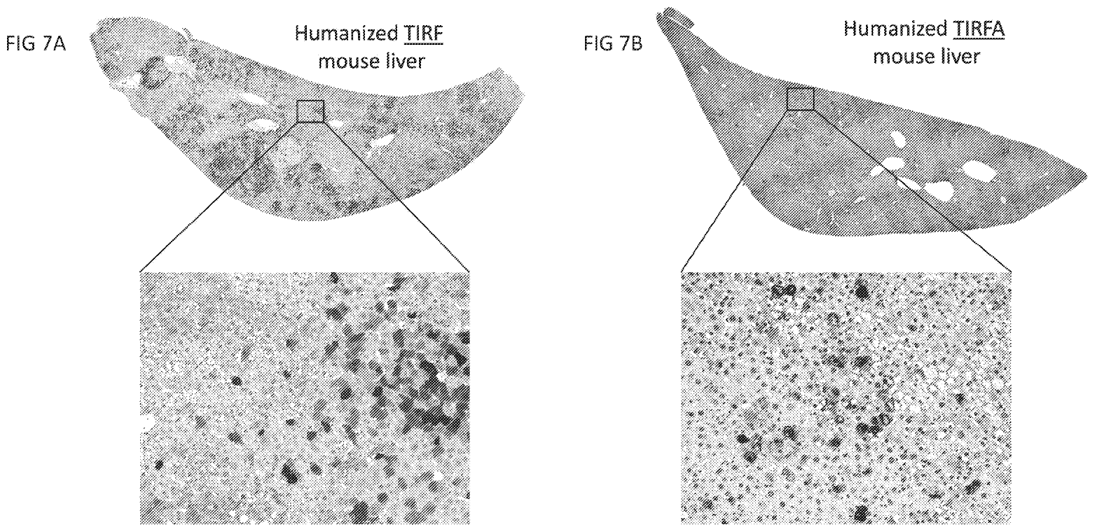

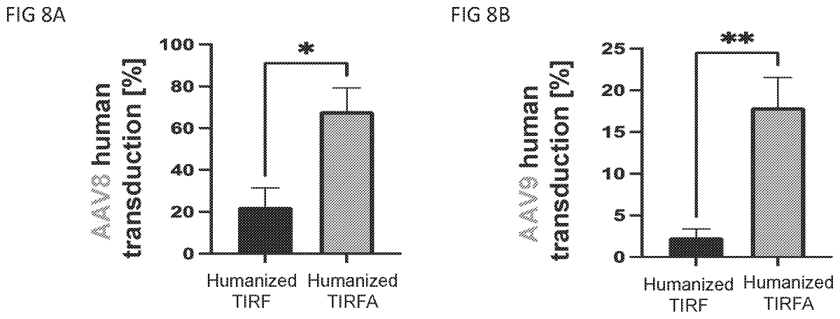

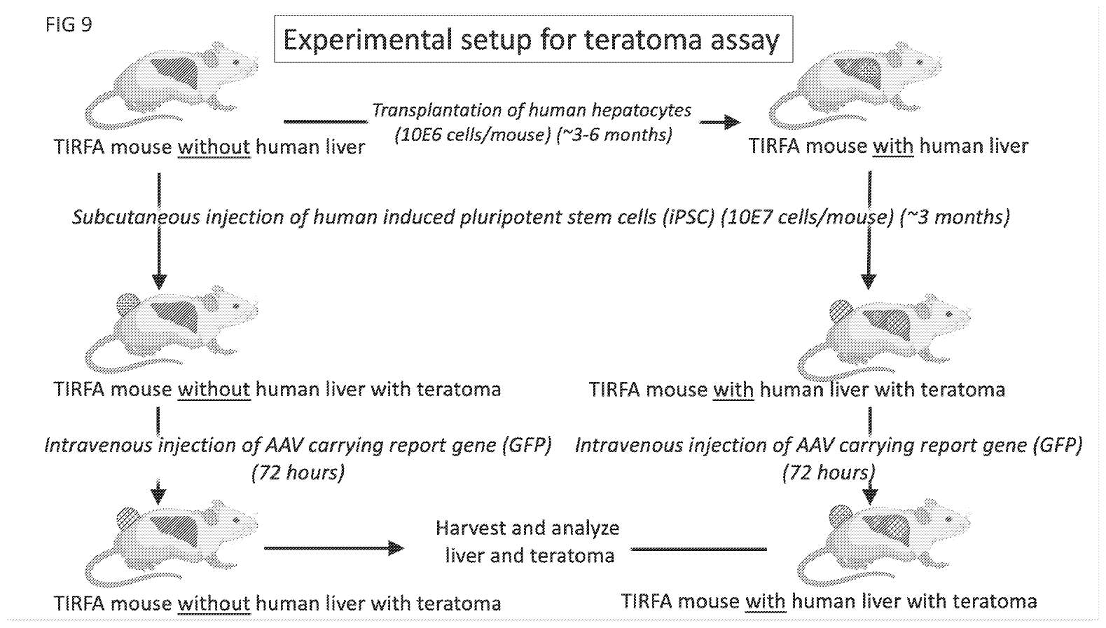

FIGS. 6A-8B show human liver chimeric TIRFA mice, e.g. TIRFA mice repopulated with human hepatocytes, which have been injected with 1×10¹² GC/mouse of AAV8 (FIGS. 6A, 6B, 8A and 8B) or AAV9 (FIGS. 7A, 7B, 8A, and 8B) carrying a td-Tomato expression cassette. Mice were euthanized 72 hours after injection, and livers harvested for staining of the human marker LDH (light grey) and the viral transgene td-Tomato (black) to show co-localization. The experiments demonstrate that AAV8 and AAV9 transduce exclusively human hepatocytes in humanized TIRFA mice, while the gene therapy vectors do not transduce murine TIRFA hepatocytes since they are deficient of the AAVR. Control humanized mice (AAVR expressing TIRF mice) demonstrate transduction of mostly murine hepatocytes and very little human hepatocytes (FIGS. 6A-7B).

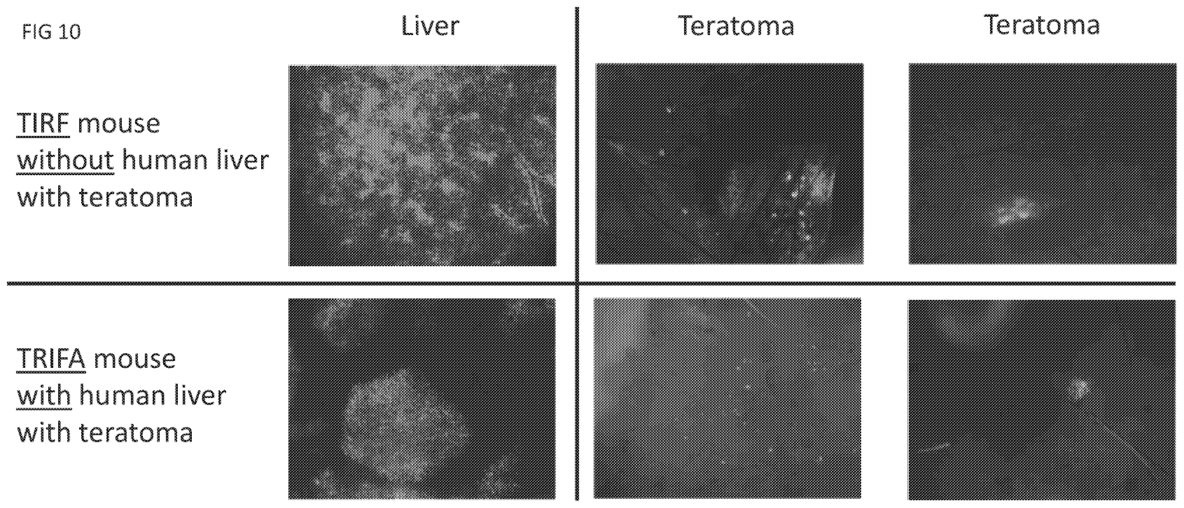

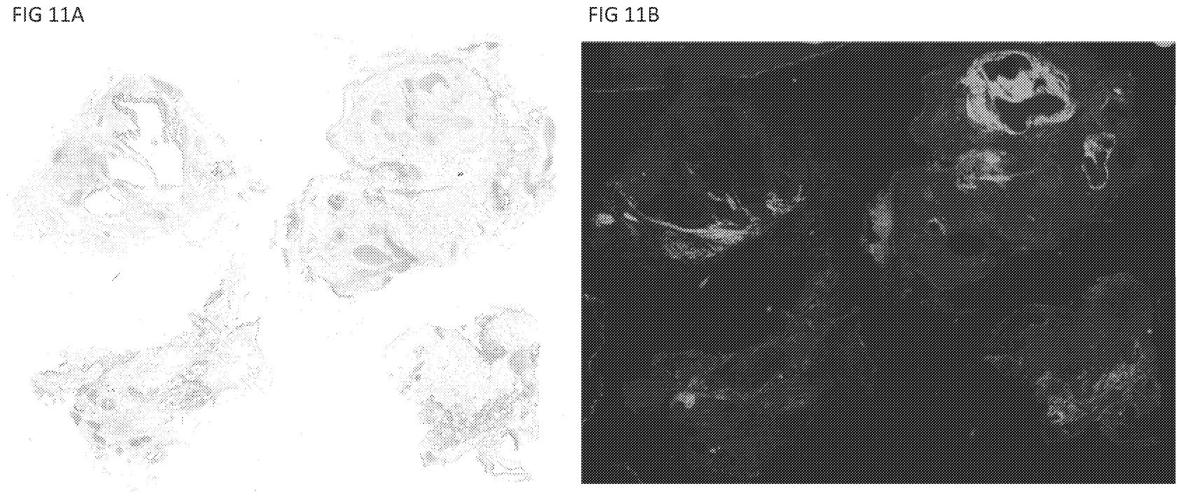

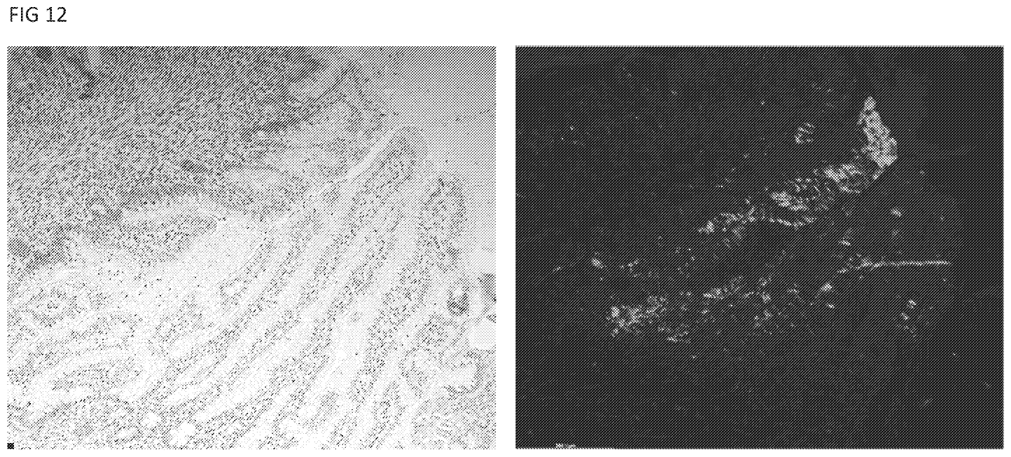

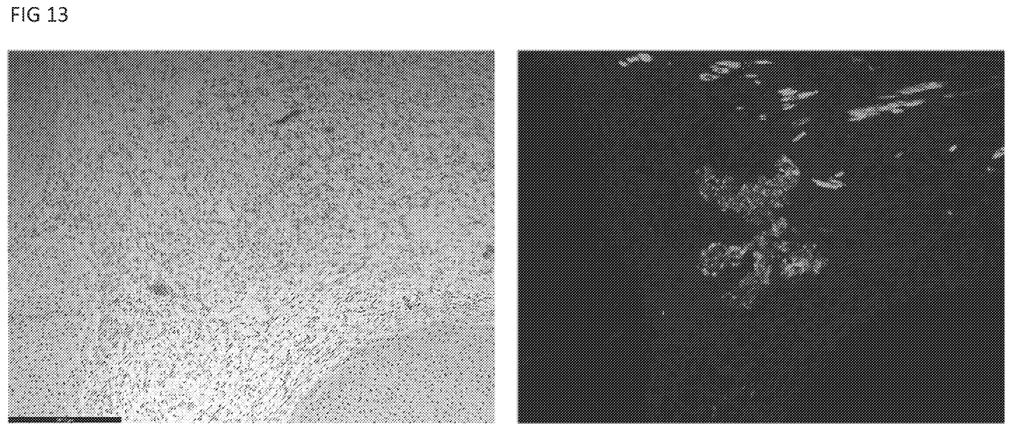

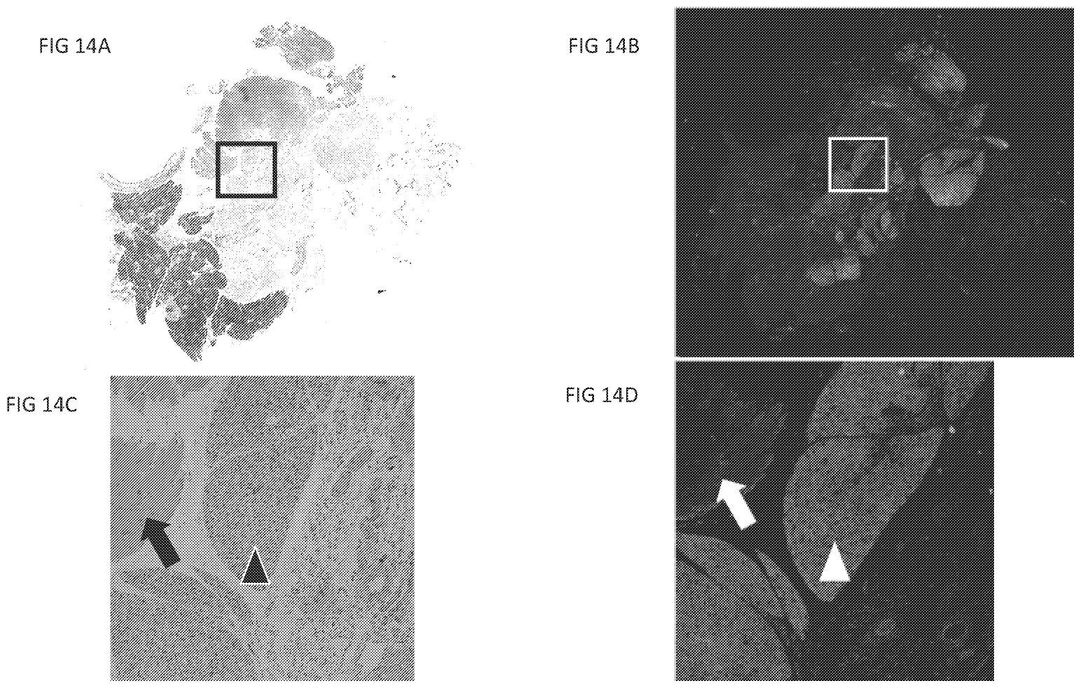

Transduction of human hepatocytes was observed in the liver of chimeric mice as well as transduction of human cells in the teratoma (FIGS. 10-14D). Noteworthy is that not all human cells in the teratoma are transduced equally well and there are significant differences in transduction efficiencies in the teratoma, e.g. different human tissues.

1. A chimeric non-human animal comprising human hepatocytes, wherein the chimeric non-human animal comprises:
a) a T-, B- and/or NK cell deficiency or functional impairment that allows re-populating with human hepatocytes to establish human chimerism in the liver of the non-human animal; and
b) a deletion or mutation of Adeno-associated virus receptor (AAVR) resulting in deficiency or functional impairment of the non-human animal AAVR.

2. A chimeric non-human animal comprising human hepatocytes,
wherein the chimeric non-human animal is a IL-2Rg^{-/-}/Rag 2^{-/-} chimeric non-human animal, and wherein the chimeric non-human animal comprises a deletion or mutation of AAVR resulting in deficiency or functional impairment of the non-human animal AAVR.

3. (canceled)

4. The chimeric non-human animal of claim 1, wherein the chimeric non-human animal is a IL-2Rg^{-/-}/Rag 2^{-/-}/Fah^{-/-} non-human animal.

5. The chimeric non-human animal of claim 1, wherein the human hepatocytes account for:
i) at least 5%;
ii) at least 10%;
iii) at least 20%;
iii) at least 30%;
iv) at least 40%;
v) at least 50%;
vi) at least 70%;
vii) at least 80%;
viii) at least 90%;
ix) at least 95%; or
x) at least 99%, of all the hepatocytes in the liver of the chimeric non-human animal.

6. A method for preparing a chimeric non-human animal comprising human hepatocytes, the method comprising:
(a) providing a T-, B- and/or NK cell deficient or impaired in function non-human animal, or providing an IL-2Rg^{-/-}/Rag2^{-/-} non-human animal, that allows re-populating with human hepatocytes to establish human chimerism in the liver of the non-human animal, wherein the non-human animal comprises a deletion or mutation of AAVR resulting in a non-functional non-human animal AAVR; and
(b) transplanting human hepatocytes into the non-human animal.

7.-10. (canceled)

11. A method of determining transduction efficiency of an AAV vector in human hepatocytes, wherein the method comprises:
(a) providing a chimeric non-human animal of claim 1;
(b) infecting the non-human animal of (a) with an amount of the AAV vector; and
(c) determining the level of transduction of the AAV vector into the human and the hepatocytes of the AAVR KO non-human animal (non-human animal hepatocytes).

12. (canceled)

13. (canceled)

14. A method of determining transduction efficiency of two or more non-identical AAV vector(s) in human hepatocytes, wherein the method comprises:
(a) providing two or more chimeric AAVR KO non-human animal of claim 1;
(b) infecting each of the non-human animals of (a) with an amount of the two or more non-identical AAV vectors, wherein one non-human animal is infected with one AAV vector;
(c) determining the level of transduction of the AAV vector into the human hepatocytes and the hepatocytes of the AAVR KO non-human animal (non-human animal hepatocytes) in each of the two or more non-human animals of (b).

15.-23. (canceled)

24. A method of determining the efficiency of a systemic AAV vector-mediated gene therapy, wherein the method comprises:
(a) providing a chimeric non-human animal of claim 1;
(b) infecting the non-human animal of (a) with an amount of an AAV vector, at least a first time;
(c) determining the level of transduction of the AAV vector into the human hepatocytes of the AAVR KO non-human animal.

25.-29. (canceled)

30. A method of determining Adeno-associated virus receptor (AAVR) dependent or AAVR-independent transduction efficiency of an AAV vector in human hepatocytes, wherein the method comprises:
(a) providing two or more non-human animals divided into two groups, Group A and Group B, each group comprising one or more non-human animals, wherein the Group A is transplanted with wild type human hepatocytes and Group B is transplanted with human hepatocytes comprising a deletion or mutation of Adeno-associated virus receptor (AAVR) resulting in a non-functional human AAVR (Hu AAVR KO human hepatocytes);
wherein the two or more non-human animals further comprise a T-, B- and/or NK cell deficiency or impairment in function, IL-2Rg−/−/Rag 2−/−/, and/or Fah−/−.
(b) infecting the one or more infected non-human animal of each group of (a) with an AAV vector;
(c) determining the level of transduction of the AAV vector into the human hepatocytes of non-human animal of Group A and Group B,
wherein the level of transduction in Group A is indicative of AAVR-dependent transduction efficiency and the level of transduction in Group B is indicative of AAVR-independent transduction efficiency, of the AAV vector.

31. (canceled)

32. A method of determining Adeno-associated virus receptor (AAVR) dependent or AAVR-independent modification and/or inhibition of an AAV vector transduction into human hepatocytes, wherein the method comprises:
(a) providing two groups of non-human animals, Group A and Group B, wherein Group A comprises one or more IL-2Rg^{-/-}/Rag 2^{-/-} non-human animals, and Group B comprises one or more IL-2Rg^{-/-}/Rag 2^{-/-} non-human animals, further comprising a deletion or mutation of Adeno-associated virus receptor (AAVR) resulting in a non-functional non-human animal AAVR;
(b) transplanting human hepatocytes into the one or more non-human animals of both groups of (a);
(c) infecting the non-human animals of both groups of (b) with an AAV vector; and
(d) determining the level of transduction of the AAV vector into the human hepatocytes and non-human hepatocytes of the non-human animals of Group A and Group B.

33. (canceled)

34. (canceled)

35. A chimeric non-human animal comprising at least two human tissues, wherein the chimeric non-human animal further comprises:
a) a T-, B- and/or NK cell deficiency or functional impairment that allows re-populating with one or more human tissues to establish human chimerism in the non-human animal; and
b) a deletion or mutation of Adeno-associated virus receptor (AAVR) resulting in deficiency or functional impairment of the non-human animal AAVR.

36. (canceled)

37. The chimeric non-human animal of claim 35, wherein the chimeric non-human animal is a IL-2Rg^{-/-}/Rag 2^{-/-}/Fah^{-/-} non-human animal.

38.-43. (canceled)

44. A method of determining Adeno-associated virus receptor (AAVR) dependent or AAVR-independent transduction efficiency of an AAV vector in one or more human tissues, wherein the method comprises:
(a) providing non-human animals divided into two groups, Group A and Group B, each group comprising one or more non-human animals, wherein the Group A is transplanted with wild type human tissue and Group B is transplanted with human tissue comprising a deletion or mutation of Adeno-associated virus receptor (AAVR) resulting in a non-functional human AAVR;
wherein the two or more non-human animals further comprise a T-, B- and/or NK cell deficiency or impairment in function, IL-2Rg−/−/Rag 2−/−/, and/or Fah−/−.
(b) infecting the one or more non-human animal of each group of (a) with an AAV vector;
(c) determining the level of transduction of the AAV vector into one or more human tissue of non-human animal of Group A and Group B,
wherein the level of transduction in Group A is indicative of AAVR-dependent transduction efficiency and the level of transduction in Group B is indicative of AAVR-independent transduction efficiency, of the AAV vector.

45.-47. (canceled)


SEQUENCE LISTING (20 sequences)

SEQ ID NO: 1 - DNA, 1540 bp, Homo sapiens
  source 1..1540: mol_type = other DNA
agaggaaacgtgtgggtggggaggggtagtgggtgagggacccaggttcctgacacagacagactacacccagggaatgaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatcaaaaaaaaaaa

SEQ ID NO: 2 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 3 - DNA, 1663 bp, Mus musculus
  source 1..1663: mol_type = other DNA
aggaaatgtatgggtggggagggcttgtgggagagtggttcagggttctgacacagactacacccagagaaagaagagcaagcaccatgttgaaactattattgtcacctagatccttcttagtccttcagctgctcctgctgagggcagggtggagctccaaggtcctcatgtccagtgcgaatgaagacatcaaagctgatttgatcctgacttctacagcccctgaacacctcagtgctcctactctgccccttccagaggttcagtgctttgtgttcaacatagagtacatgaattgcacttggaatagcagttctgagcctcaggcaaccaacctcacgctgcactataggtacaaggtatctgataataatacattccaggagtgcagtcactatttgttctccaaagagattacttctggctgtcagatacaaaaagaagatatccagctctaccagacatttgttgtccagctccaggacccccagaaaccccagaggcgagctgtacagaagctaaacctacagaatcttgtgatcccacgggctccagaaaatctaacactcagcaatctgagtgaatcccagctagagctgagatggaaaagcagacatattaaagaacgctgtttacaatacttggtgcagtaccggagcaacagagatcgaagctggacggaactaatagtgaatcatgaacctagattctccctgcctagtgtggatgagctgaaacggtacacatttcgggttcggagccgctataacccaatctgtggaagttctcaacagtggagtaaatggagccagcctgtccactgggggagtcatactgtagaggagaatccttccttgtttgcactggaagctgtgcttatccctgttggcaccatggggttgattattaccctgatctttgtgtactgttggttggaacgaatgcctccaattccccccatcaagaatctagaggatctggttactgaataccaagggaacttttcggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctcaaagtcctatagtcctaagtgacgctaacctcccctactcaccttggcaatctggatccaatgctcactgccttcccttggggctaagtttcgatttcctgtcccatgtaactgcttttctgttccatatgccctacttgagagtgtcccttgccctctttccctgcacaagccctcccatgcccagcctaacacctttccactttctttgaagagagtcttaccctgtagcccagggtggctgggagctcactatgtaggccaggttggcctccaactcacaggctatcctcccacctctgcctcataagagttggggttactggcatgcaccaccacacccagcatggtccttctcttttataggattctccctccctttttctacctatgattcaactgtttccaaatcaacaagaaataaagtttttaaccaatgatca

SEQ ID NO: 4 - protein, 369 aa, Mus musculus
MLKLLLSPRSFLVLQLLLLRAGWSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 5 - DNA, 2457 bp, Homo sapiens
  source 1..2457: mol_type = other DNA
attagatcagtgttcataagaacatctgtaggcacacatacacactctctttacagtcagccttctgcttgccacagtcatagtgggcagtcagtgaatcttccccaagtgctgacaattaatacctggtttagcggcaaagattcagagaggcgtgagcagcccctctggccttcagacaaaaatctacgtaccatcagaaactatgtctctgcagatggtaacagtcagtaataacatagccttaattcagccaggcttctcactgatgaattttgatggacaagttttcttctttggacaaaaaggctggcccaaaagatcctgccccactggagttttccatctggatgtaaagcataaccatgtcaaactgaagcctacaattttctctaaggattcctgctacctccctcctcttcgctacccagccacttgcacattcaaaggcagcttggagtctgaaaagcatcaatacatcatccatggagggaaaacaccaaacaatgaggtttcagataagatttatgtcatgtctattgtttgcaagaacaacaaaaaggttacttttcgctgcacagagaaagacttggtaggagatgttcctgaagccagatatggtcattccattaatgtggtgtacagccgagggaaaagtatgggtgttctctttggaggacgctcatacatgccttctacccacagaaccacagaaaaatggaatagtgtagctgactgcctgccctgtgttttcctggtggattttgaatttgggtgtgctacatcatacattcttccagaacttcaggatgggctatcttttcatgtctctattgccaaaaatgacaccatctatattttaggaggacattcacttgccaataatatccggcctgccaacctgtacagaataagggttgatcttcccctgggtagcccagctgtgaattgcacagtcttgccaggaggaatctctgtctccagtgcaatcctgactcaaactaacaatgatgaatttgttattgttggtggctatcagcttgaaaatcaaaaaagaatgatctgcaacatcatctctttagaggacaacaagatagaaattcgtgagatggagaccccagattggaccccagacattaagcacagcaagatatggtttggaagcaacatgggaaatggaactgtttttcttggcataccaggagacaataaacaagttgtttcagaaggattctatttctatatgttgaaatgtgctgaagatgatactaatgaagagcagacaacattcacaaacagtcaaacatcaacagaagatccaggggattccactccctttgaagactctgaagaattttgtttcagtgcagaagcaaatagttttgatggtgatgatgaatttgacacctataatgaagatgatgaagaagatgagtctgagacaggctactggattacatgctgccctacttgtgatgtggatatcaacacttgggtaccattctattcaactgagctcaacaaacccgccatgatctactgctctcatggggatgggcactgggtccatgctcagtgcatggatctggcagaacgcacactcatccatctgtcagcaggaagcaacaagtattactgcaatgagcatgtggagatagcaagagctctacacactccccaaagagtcctacccttaaaaaagcctccaatgaaatccctccgtaaaaaaggttctggaaaaatcttgactcctgccaagaaatcctttcttagaaggttgtttgattagttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaaaaaaaaaaaaaaaaaaaaaaaaaaaa

SEQ ID NO: 6 - protein, 527 aa, Homo sapiens
MSLQMVTVSNNIALIQPGFSLMNFDGQVFFFGQKGWPKRSCPTGVFHLDVKHNHVKLKPTIFSKDSCYLPPLRYPATCTFKGSLESEKHQYIIHGGKTPNNEVSDKIYVMSIVCKNNKKVTFRCTEKDLVGDVPEARYGHSINVVYSRGKSMGVLFGGRSYMPSTHRTTEKWNSVADCLPCVFLVDFEFGCATSYILPELQDGLSFHVSIAKNDTIYILGGHSLANNIRPANLYRIRVDLPLGSPAVNCTVLPGGISVSSAILTQTNNDEFVIVGGYQLENQKRMICNIISLEDNKIEIREMETPDWTPDIKHSKIWFGSNMGNGTVFLGIPGDNKQVVSEGFYFYMLKCAEDDTNEEQTTFTNSQTSTEDPGDSTPFEDSEEFCFSAEANSFDGDDEFDTYNEDDEEDESETGYWITCCPTCDVDINTWVPFYSTELNKPAMIYCSHGDGHWVHAQCMDLAERTLIHLSAGSNKYYCNEHVEIARALHTPQRVLPLKKPPMKSLRKKGSGKILTPAKKSFLRRLFD

SEQ ID NO: 7 - DNA, 3393 bp, Mus musculus
  source 1..3393: mol_type = other DNA
actctaccctgcagccttcagcttggcacaaactaaacagtgactcttccccaagtgccgagtttaattcctggcttggccgaaaggattcagagagggataagcagcccctctggccttcagtgccaaaataagaaagagtatttcacatccacaagcaggaagtacacttcatacctctctaagataaaagacctattcacaatcaaaaatgtccctgcagatggtaacagtgggtcataacatagccttaattcaaccaggcttctcacttatgaattttgatggccaagttttcttctttggccagaaaggctggcctaagagatcctgtcctactggagtctttcattttgatataaaacaaaatcatctcaaactgaagcctgcaatcttctctaaagattcctgctacctcccacctcttcgttatccagctacttgctcatacaaaggcagcatagactctgacaagcatcaatatatcattcacggagggaaaacaccaaacaatgagctttccgataagatttatatcatgtctgtcgcttgcaagaataacaaaaaagttactttccgttgcacagagaaagacttagtaggagatgtccctgaacccagatacggccattccattgacgtggtgtatagtcgagggaaaagcatgggtgttctctttggaggacgttcatacatgccttctacccagagaaccacagaaaaatggaatagtgtagctgactgcctaccccatgttttcttgatagattttgaatttgggtgtgctacatcatatattctcccagaacttcaggatgggctgtcttttcatgtttctattgccagaaacgataccgtttatattttgggaggacactcacttgccagtaatatacgccctgctaacttgtatagaataagagtggaccttcccctgggtaccccagcagtgaattgcacagtcttgccaggaggaatctctgtctccagtgcaatcctcactcaaacaaacaatgatgaatttgttattgtgggtggttatcagctggaaaatcagaaaaggatggtctgcagccttgtctctctaggggacaacacgattgaaatcagtgagatggagactcctgactggacctcagatattaagcatagcaaaatatggtttggaagcaacatgggaaacgggactattttccttggcataccaggagacaataagcaggctatgtcagaagcattctatttctatactttgagatgctctgaagaggatttgagtgaagatcagaaaattgtctccaacagtcagacatcaacagaagatcctggggactccactccctttgaagactcagaggaattttgtttcagtgctgaagcaaccagttttgatggtgacgatgaatttgacacctacaatgaagatgatgaagatgacgagtctgtaaccggctactggataacatgttgccctacttgtgatgttgacatcaatacctgggttccgttctattcaacggagctcaataaacccgccatgatctattgttctcatggggatgggcactgggtacatgcccagtgcatggatttggaagaacgcacactcatccacttgtcagaaggaagcaacaagtattattgcaatgaacatgtacagatagcaagagcattgcaaactcccaaaagaaacccccccttacaaaaacctccaatgaaatccctccacaaaaaaggctctgggaaagtcttgactcctgccaagaaatccttccttagaagactgtttgattaatttagcaaaagcccctcagactcaggtatattgctctctgaatctactttcaatcataaacattattttgatttttgtttactgaaatctctatgttatgttttagttatgtgaattaagtgctgttgtgatttattgttaagtataactattctaatgtgtgttttttaacatcttatccaggaatgtcttaaatgagaaatgttatacagttttccattaaggatatcagtgataaagtatagaactcttacattattttgtaacaatctacatattgaatagtaactaaataccaataaataaactaatgcacaaaaagttaagttcttttgtgtaataagtagcctatagttggtttaaacagttaaaaccaacagctatatcccacactactgctgtttataaattttaaggtggcctctggtttatacttatgagcagaattatatatattggtcaataccatgaagaaaaatttaattctatatcaagccaggcatggtgatggtgatacatgcctgtaatcctggcacttaggaagtggaagaaggaagtttgtgagtttgatgcttgttgaggtatgaccttttgctatgtattgtagtgtatgagccccaagacctgcttgacccagagacaagagagtccacacatagatccaagtaatgctatgtgaccttgccccccggttacttgtgattaggtgaataaagatgtcaacagccaatagctgggcagaagagccaaaagtggggattgagggtaccctggcttgatgtaggaggagaccatgaggaaaggggagaaaaaagtgatggaggaggagaaagatgccatgagctaggagttaagaaagcatggccatgagtgctggccaattggagttaagagcagcccagatgaaacatagtaagtaataactcagggttatcgatagaaaatagattttagtgccgtactctccccagccctagagctgactatggcttactgtaaatataaagtttgtatgtgtcttttatccaggaactaaatggtcaaaggtggagtagaaactctggattgggattaaatttttctacaacaaatgctggcctgggctagattttatctcatatccgaaggctgacagaacacagagcactggtaacattgccacctgccatgcacaaagacctgagtctaatactgtggacattttcttgaagtatctacatgtacttctggagtgaaaacatattccaacaatatgcctttgtttaaatcactcactcactttgggccctcacattatatcctttcaaaatcaatggttcacccctttgaaaatgcttagccatagtccctcatcttccttaaagacagttgtcatctctggaaatagtcacatgtcattcaaggtccaatactgtgcagctctgaagtatggcattaccactttaagtgaaaagtgaaatatgaacatgagctcagacaaaggtttgggactatcactctcaaggaggctctactgctaagtcctgaactgctttcacatgaatacagaaattataacaaaaaatatgtaatcaataaaaagaaaactttcatattcc

SEQ ID NO: 8 - protein, 527 aa, Mus musculus
MSLQMVTVGHNIALIQPGFSLMNFDGQVFFFGQKGWPKRSCPTGVFHFDIKQNHLKLKPAIFSKDSCYLPPLRYPATCSYKGSIDSDKHQYIIHGGKTPNNELSDKIYIMSVACKNNKKVTFRCTEKDLVGDVPEPRYGHSIDVVYSRGKSMGVLFGGRSYMPSTQRTTEKWNSVADCLPHVFLIDFEFGCATSYILPELQDGLSFHVSIARNDTVYILGGHSLASNIRPANLYRIRVDLPLGTPAVNCTVLPGGISVSSAILTQTNNDEFVIVGGYQLENQKRMVCSLVSLGDNTIEISEMETPDWTSDIKHSKIWFGSNMGNGTIFLGIPGDNKQAMSEAFYFYTLRCSEEDLSEDQKIVSNSQTSTEDPGDSTPFEDSEEFCFSAEATSFDGDDEFDTYNEDDEDDESVTGYWITCCPTCDVDINTWVPFYSTELNKPAMIYCSHGDGHWVHAQCMDLEERTLIHLSEGSNKYYCNEHVQIARALQTPKRNPPLQKPPMKSLHKKGSGKVLTPAKKSFLRRLFD

SEQ ID NO: 9 - DNA, 1810 bp, Homo sapiens
  source 1..1810: mol_type = other DNA
gagaccaaaagtcaggtaggagcctccggggtccctgctgtgtcacccggacaggccgtgggggcgggcaggggggcggggccgggcctgaccacagcggccgagttcagtcctgctctccgcacgccaccttaggcccgcagccgtgccgggtgctcttcagcatgtccttcatcccggtggccgaggattccgacttccccatccacaacctgccctacggcgtcttctcgaccagaggcgacccaagaccgaggataggtgtggccattggcgaccagatcctggacctcagcatcatcaagcacctctttactggtcctgtcctctccaaacaccaggatgtcttcaatcagcctacactcaacagcttcatgggcctgggtcaggctgcctggaaggaggcgagagtgttcttgcagaacttgctgtctgtgagccaagccaggctcagagatgacaccgaacttcggaagtgtgcattcatctcccaggcttctgccacgatgcaccttccagccaccataggagactacacagacttctattcctctcggcagcatgctaccaacgtcggaatcatgttcagggacaaggagaatgcgttgatgccaaattggctgcacttaccagtgggctaccatggccgtgcctcctctgtcgtggtgtctggcaccccaatccgaaggcccatgggacagatgaaacctgatgactctaagcctcccgtatatggtgcctgcaagctcttggacatggagctggaaatggctttttttgtaggccctggaaacagattgggagagccgatccccatttccaaggcccatgagcacatttttggaatggtccttatgaacgactggagtgcacgagacattcagaagtgggagtatgtccctctcgggccattccttgggaagagttttgggaccactgtctctccgtgggtggtgcccatggatgctctcatgccctttgctgtgcccaacccgaagcaggaccccaggcccctgccgtatctgtgccatgacgagccctacacatttgacatcaacctctctgttaacctgaaaggagaaggaatgagccaggcggctaccatatgcaagtccaattttaagtacatgtactggacgatgctgcagcagctcactcaccactctgtcaacggctgcaacctgcggccgggggacctcctggcttctgggaccatcagcgggccggagccagaaaacttcggctccatgttggaactgtcgtggaagggaacgaagcccatagacctggggaatggtcagaccaggaagtttctgctggacggggatgaagtcatcataacagggtactgccagggggatggttaccgcatcggctttggccagtgtgctggaaaagtgctgcctgctctcctgccatcatgagattttctctgctcttctggaaacaaagggctcaagcacccctttcaaccctgtgactggggtcctccctcgggctgtaggcctggtccgccattcagtgacaaataaagccattgtgctctgaggcctgcactgccgcagatgcagctgtgtccacttatgatcgtgatttgatccagtgggtcaaggtgtgtaaagcctccctgccagatattcattaatatgttttctcactcttattagtgaggtcaggggtctttgtgggattttcttattagacatcccaggcctcctggtattccatggaatttgaaaagagactggcacctgtagtagtcagggctctccagagaaatagaaccaaggagaaagaaaaaaaaaaaaaa

SEQ ID NO: 10 - protein, 419 aa, Homo sapiens
MSFIPVAEDSDFPIHNLPYGVFSTRGDPRPRIGVAIGDQILDLSIIKHLFTGPVLSKHQDVFNQPTLNSFMGLGQAAWKEARVFLQNLLSVSQARLRDDTELRKCAFISQASATMHLPATIGDYTDFYSSRQHATNVGIMFRDKENALMPNWLHLPVGYHGRASSVVVSGTPIRRPMGQMKPDDSKPPVYGACKLLDMELEMAFFVGPGNRLGEPIPISKAHEHIFGMVLMNDWSARDIQKWEYVPLGPFLGKSFGTTVSPWVVPMDALMPFAVPNPKQDPRPLPYLCHDEPYTFDINLSVNLKGEGMSQAATICKSNFKYMYWTMLQQLTHHSVNGCNLRPGDLLASGTISGPEPENFGSMLELSWKGTKPIDLGNGQTRKFLLDGDEVIITGYCQGDGYRIGFGQCAGKVLPALLPS

SEQ ID NO: 11 - DNA, 1597 bp, Mus musculus
  source 1..1597: mol_type = other DNA
gggtgctaaaagaatcactagggtggggaggcggtcccagtggggcgggtaggggtgtgtgccaggtggtaccgggtattggctggaggaagggcagcccggggttcggggcggtccctgaatctaaaggccctcggctagtctgatccttgccctaagcatagtcccgttagccaaccccctacccgccgtgggctctgctgcccggtgctcgtcagcatgtcctttattccagtggccgaggactccgactttcccatccaaaacctgccctatggtgttttctccactcaaagcaacccaaagccacggattggtgtagccatcggtgaccagatcttggacctgagtgtcattaaacacctctttaccggacctgccctttccaaacatcaacatgtcttcgatgagacaactctcaataacttcatgggtctgggtcaagctgcatggaaggaggcaagagcatccttacagaacttactgtctgccagccaagcccggctcagagatgacaaggagcttcggcagcgtgcattcacctcccaggcttctgcgacaatgcaccttcctgctaccataggagactacacggacttctactcttctcggcagcatgccaccaatgttggcattatgttcagaggcaaggagaatgcgctgttgccaaattggctccacttacctgtgggataccatggccgagcttcctccattgtggtatctggaaccccgattcgaagacccatggggcagatgagacctgataactcaaagcctcctgtgtatggtgcctgcagactcttagacatggagttggaaatggctttcttcgtaggccctgggaacagattcggagagccaatccccatttccaaagcccatgaacacattttcgggatggtcctcatgaacgactggagcgcacgagacatccagcaatgggagtacgtcccacttgggccattcctggggaaaagctttggaaccacaatctccccgtgggtggtgcctatggatgccctcatgccctttgtggtgccaaacccaaagcaggaccccaagcccttgccatatctctgccacagccagccctacacatttgatatcaacctgtctgtctctttgaaaggagaaggaatgagccaggcggctaccatctgcaggtctaactttaagcacatgtactggaccatgctgcagcaactcacacaccactctgttaatggatgcaacctgagacctggggacctcttggcttctggaaccatcagtggatcagaccctgaaagctttggctccatgctggaactgtcctggaagggaacaaaggccatcgatgtggagcaggggcagaccaggaccttcctgctggacggcgatgaagtcatcataacaggtcactgccagggggacggctaccgtgttggctttggccagtgtgctgggaaagtgctgcctgccctttcaccagcctgaagctccggaagtcacaagacacacccttgccttatgaggatcatgctaccactgcatcagtcaggaatgaataaagctactttgattgtgggaaatgccacagaaaaaaaaaaaaaaa

SEQ ID NO: 12 - protein, 419 aa, Mus musculus
MSFIPVAEDSDFPIQNLPYGVFSTQSNPKPRIGVAIGDQILDLSVIKHLFTGPALSKHQHVFDETTLNNFMGLGQAAWKEARASLQNLLSASQARLRDDKELRQRAFTSQASATMHLPATIGDYTDFYSSRQHATNVGIMFRGKENALLPNWLHLPVGYHGRASSIVVSGTPIRRPMGQMRPDNSKPPVYGACRLLDMELEMAFFVGPGNRFGEPIPISKAHEHIFGMVLMNDWSARDIQQWEYVPLGPFLGKSFGTTISPWVVPMDALMPFVVPNPKQDPKPLPYLCHSQPYTFDINLSVSLKGEGMSQAATICRSNFKHMYWTMLQQLTHHSVNGCNLRPGDLLASGTISGSDPESFGSMLELSWKGTKAIDVEQGQTRTFLLDGDEVIITGHCQGDGYRVGFGQCAGKVLPALSPA

SEQ ID NO: 13 - protein, 1048 aa, Mus musculus
MEKRLGVKPSPASWVLPGYCWQTSVKLPRSLYLLYSFFCFSVLWLSTDADESRCQQGKTLYGAGLRTEGENHLRLLAGSLPFHACRAACCRDSACHALWWLEGMCFQADCSKPQSCQPFRTDSSNSMLIIFQKSQTTDDLGLLPEDDEPHLLRLGWGRTSWRRQSLLGAPLTLSVPSSHHQSLLRDRQKRDLSVVPTHGAMQHSKVNHSEEAGALSPTSAEVRKTITVAGSFTSNHTTQTPEWPKNVSIHPEPSEHSSPVSGTPQVKSTEHSPTDAPLPVAPSYSYATPTPQASSQSTSAPHPVVKELVVSAGKSVQITLPKNEVQLNAFVLPEAEPGETYTYDWQLITHPTDYSGEVERKHSQSLQLSKLTPGLYEFKVTVDGQNAHGEGYVNVTVKPEPRKNRPPVAVVSPQFQEISLPTTSTIIDGSQSTDDDKIVQYHWEELKGPLREEKISEDTAILKLSKLVPGNYTFSLTVVDSDGATNSTTASLTVNKAVDYPPVANAGPNQVITLPQNSITLFGNQSTDDHGITSYEWSLSPSSKGKVVEMQGVRTPALQLSAMQEGDYTYQLTVTDTAGQQATAQVTVIVQPENNKPPQADAGPDKELTLPVDSTTLDGSKSTDDQRVVSYLWEQSRGPDGVQLENANSSVATVTGLQVGTYVFTLTVKDERNLQSQSSVNVIVKEEINKPPVAKIAGNVVVTLPTSTAELDGSRSSDDKGIVSYLWTRDETSPAAGEVLNHSDHHPVLFLSNLVEGTYTFHLKVTDAKGESDTDRTTVEVKPDPRKSNLVEIILDVNVSQLTERLKGMLIRQIGVLLGVLDSDIIVQKIQPYTEQSTKMLFFVQNDPPHQLFKGHEVAAMLKSELQKQKADFLIFRALEISTVTCQLNCSDHGHCDSFTKRCVCDPFWMENFIKVQLRDGDSNCEWSVLYVIIASFVIVVALGILSWTTICCCKRQKGKPKRKSRYKILDATDQESLELKPTSRAGSKQKGPTLSSSLMHSESELDSDDAIFTWPDREKGKLLYGQNGSVPNGQTPLKSRSAREEIL

SEQ ID NO: 14 - protein, 1049 aa, Homo sapiens
MEKRLGVKPNPASWILSGYYWQTSAKWLRSLYLFYTCFCFSVLWLSTDASESRCQQGKTQFGVGLRSGGENHLWLLEGTPSLQSCWAACCQDSACHVFWWLEGMCIQADCSRPQSCRAFRTHSSNSMLVFLKKFQTADDLGFLPEDDVPHLLGLGWNWASWRQSPPRAALRPAVSSSDQQSLIRKLQKRGSPSDVVTPIVTQHSKVNDSNELGGLTTSGSAEVHKAITISSPLTTDLTAELSGGPKNVSVQPEISEGLATTPSTQQVKSSEKTQIAVPQPVAPSYSYATPTPQASFQSTSAPYPVIKELVVSAGESVQITLPKNEVQLNAYVLQEPPKGETYTYDWQLITHPRDYSGEMEGKHSQILKLSKLTPGLYEFKVIVEGQNAHGEGYVNVTVKPEPRKNRPPIAIVSPQFQEISLPTTSTVIDGSQSTDDDKIVQYHWEELKGPLREEKISEDTAILKLSKLVPGNYTFSLTVVDSDGATNSTTANLTVNKAVDYPPVANAGPNQVITLPQNSITLFGNQSTDDHGITSYEWSLSPSSKGKVVEMQGVRTPTLQLSAMQEGDYTYQLTVTDTIGQQATAQVTVIVQPENNKPPQADAGPDKELTLPVDSTTLDGSKSSDDQKIISYLWEKTQGPDGVQLENANSSVATVTGLQVGTYVFTLTVKDERNLQSQSSVNVIVKEEINKPPIAKITGNVVITLPTSTAELDGSKSSDDKGIVSYLWTRDEGSPAAGEVLNHSDHHPILFLSNLVEGTYTFHLKVTDAKGESDTDRTTVEVKPDPRKNNLVEIILDINVSQLTERLKGMFIRQIGVLLGVLDSDIIVQKIQPYTEQSTKMVFFVQNEPPHQIFKGHEVAAMLKSELRKQKADFLIFRALEVNTVTCQLNCSDHGHCDSFTKRCICDPFWMENFIKVQLRDGDSNCEWSVLYVIIATFVIVVALGILSWTVICCCKRQKGKPKRKSKYKILDATDQESLELKPTSRAGIKQKGLLLSSSLMHSESELDSDDAIFTWPDREKGKLLHGQNGSVPNGQTPLKARSPREEIL

SEQ ID NO: 15 - RNA, 23 nt, synthetic construct
  source 1..23: mol_type = other RNA
acttgcggagtaccagatacagg

SEQ ID NO: 16 - RNA, 23 nt, synthetic construct
  source 1..23: mol_type = other RNA
cattcttaggcagggtgatctgg

SEQ ID NO: 17 - DNA, 20 bp, synthetic construct
  source 1..20: mol_type = other DNA
acagttgccggttccttcac

SEQ ID NO: 18 - DNA, 21 bp, synthetic construct
  source 1..21: mol_type = other DNA
ccacatgcacatcacaacctc

SEQ ID NO: 19 - DNA, 25 bp, synthetic construct
  source 1..25: mol_type = other DNA
aacatgctacgcagagagggagtgg

SEQ ID NO: 20 - DNA, 29 bp, synthetic construct
  source 1..29: mol_type = other DNA
catgagacaaggaacccctagtgatggag